SEQUENCE LISTING (17 sequences)

SEQ ID NO: 1 - DNA, 4242 bp, Artificial sequence
  Nucleotide sequence of plasmid pGCMB75.6
tgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgcaagcttgatatcgaattcctgcagcccgggggatccactagttctagagcggccgccaccgcggtggagctcgaattatcagatcgattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctaggcttttgcaaagatcgatcaagagacaggatgaggatcgtttcgcagcttttcattctgactgcaacgggcaataagtctctgtgtggattaaaaaaagagtgtctgatagcagcttctgaactggttacctgccgtgagtaaattaaaattttattgacttaggtcactaaggcgccttgcgctgaggttgcgtcgtgatatcatcagggcagaccggttacatccccctaacaagctgtataaagagaaatactatctcattggcgttgcccgcacctgacagtgcgacgttgggctgcgtccgtcgaccaacggtaccgaggtaacagcccaatctatccatgatctcggccaggccgggtcggccgttatgcagcccggctcgggtatgaagccattaaggagccgacccagcgcgaccgggcggccggtcacgctgcctctgctgaagcctgcctgtcactccctgcgcggcgtacccgccgttctcatcgagtaggctccggatcgcgaccccggacgggccctgggcccaggagcggcctatgacaaatgccgggtagcgatccggcattcagcattgactgcgcacggatccagtccttgcaggagccttatgccgaccgtagcaaaaaatgagcccgagccgatcgcgagttgtgatccggtcccgccgattgccggtcgcgatgacggtcctgtgtaagcgttatcgttaccaattgtttaagaagtatatacgctacgaggtacttgataacttctgcgtagcatacatgaggttttgtataaaaatggcgggcgatatcaacgcagtgtcagaaatccgaaacagtctgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggc

SEQ ID NO: 2 - DNA, 4242 bp, Artificial sequence
  Nucleotide sequence of plasmid pMB75.6
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgcaagcttgatatcgaattcctgcagcccgggggatccactagttctagagcggccgccaccgcggtggagctcgaattatcagatcgattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcgcgcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccac

SEQ ID NO: 3 - DNA, 4242 bp, Artificial sequence
  Nucleotide sequence of plasmid pMB75.6_AscI
tgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgcaagcttgatatcgaattcctgcagcccgggggatccactagttctagagcggccgccaccgcggtggagctcgaattatcagatcgattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggc

SEQ ID NO: 4 - DNA, 4242 bp, Artificial sequence
  Nucelotide sequence of plasmid pLacZMB75.6
tgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgccaaaacaaaattcctcaaaaatcatcatcgaatgaatggtgaaataatttccctgaataactgtagtgttttcagggcgcggcataataattaactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctaggcttttgcaaagatcgatcaagagacaggatgaggatcgtttcgcagcttttcattctgactgcaacgggcaataagtctctgtgtggattaaaaaaagagtgtctgatagcagcttctgaactggttacctgccgtgagtaaattaaaattttattgacttaggtcactaaggcgccttgcgctgaggttgcgtcgtgatatcatcagggcagaccggttacatccccctaacaagctgtataaagagaaatactatctcattggcgttgcccgcacctgacagtgcgacgttgggctgcgtccgtcgaccaacggtaccgaggtaacagcccaatctatccatgatctcggccaggccgggtcggccgttatgcagcccggctcgggtatgaagccattaaggagccgacccagcgcgaccgggcggccggtcacgctgcctctgctgaagcctgcctgtcactccctgcgcggcgtacccgccgttctcatcgagtaggctccggatcgcgaccccggacgggccctgggcccaggagcggcctatgacaaatgccgggtagcgatccggcattcagcattgactgcgcacggatccagtccttgcaggagccttatgccgaccgtagcaaaaaatgagcccgagccgatcgcgagttgtgatccggtcccgccgattgccggtcgcgatgacggtcctgtgtaagcgttatcgttaccaattgtttaagaagtatatacgctacgaggtacttgataacttctgcgtagcatacatgaggttttgtataaaaatggcgggcgatatcaacgcagtgtcagaaatccgaaacagtctgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgc

SEQ ID NO: 5 - DNA, 20 bp, Artificial sequence
  Primer
gtgcgcggaacccctatttg

SEQ ID NO: 6 - DNA, 20 bp, Artificial sequence
  Primer
gcgtacccgccgttctcatc

SEQ ID NO: 7 - DNA, 20 bp, Artificial sequence
  Primer
gtctgacgctcagtggaacg

SEQ ID NO: 8 - DNA, 22 bp, Artificial sequence
  Primer
gttccagtttggaacaagagtc

SEQ ID NO: 9 - DNA, 20 bp, Artificial sequence
  Primer
ggcaattagccatattagtc

SEQ ID NO: 10 - DNA, 22 bp, Artificial sequence
  Primer
gcagagctcgtttagtgaaccg

SEQ ID NO: 11 - DNA, 21 bp, Artificial sequence
  Primer
gatcataatcagccataccac

SEQ ID NO: 12 - DNA, 27 bp, Artificial sequence
  Primer
cgtcttgagtccaacccggtaagacac

SEQ ID NO: 13 - DNA, 24 bp, Artificial sequence
  Primer
ccacaggtgtccactcccaggttc

SEQ ID NO: 14 - DNA, 21 bp, Artificial sequence
  Primer
ctagtcaaggcactatacatc

SEQ ID NO: 15 - DNA, 22 bp, Artificial sequence
  Primer
tccacagaatcaggggataacg

SEQ ID NO: 16 - DNA, 23 bp, Artificial sequence
  Primer
gctcactcattaggcaccccagg

SEQ ID NO: 17 - DNA, 22 bp, Artificial sequence
  Primer
cgcgtaatacgactcactatag

(19) United States
(12) Patent Application Publication
(10) Pub. No.: US 2019/0233825 A1
(43) Pub. Date: Aug. 1, 2019

(54) METHODS OF MODULATING CYTOSOLIC DNA SURVEILLANCE MOLECULES

(71) Applicant: BAYER ANIMAL HEALTH GMBH, Leverkusen (DE)

(72) Inventors: Thomas ILG, Monheim (DE); Albert ABRAHAM, Shawnee, KS (US); Jason NICKELL, Parkville, MO (US); Daniel KEIL, Spring Hill, KS (US); Christian WEISS, Leverkusen (DE)

(21) Appl. No.: 15/738,794

(22) PCT Filed: Jun. 23, 2016
(86) PCT No.: PCT/EP2016/064613

§ 371 Date: Dec. 21, 2017

Related U.S. Application Data
(60) Provisional application No. 62/185,230, filed on Jun. 26, 2015.

(51) Int. Cl. C12N 15/117; A61K 9/127; A61K 39/39; A61P 37/04; A61K 31/4709; A61K 31/472

(52) U.S. Cl. CPC C12N 15/117; A61K 9/127; A61K 39/39; A61K 2039/552; A61K 31/4709; A61K 31/472; A61P 37/04

(57) ABSTRACT

The present invention generally relates to methods of eliciting an immune response in a subject by activating specific innate immunity signaling molecules and pathway. In particular, an immunomodulator composition is used to stimulate innate immunity signaling molecules and pathways.

### CROSS-REFERENCE

This application claims the benefit under 35 U.S.C § 119(e) of Provisional U.S. Patent Application No. 62/185,230, filed on Jun. 26, 2015, and entitled “METHODS OF MODULATING CYTOSOLIC DNA SURVEILLANCE MOLECULES,” the content of which is incorporated by reference herein in its entirety.

### FIELD OF THE INVENTION

The present invention generally relates to methods of eliciting an immune response in a subject by activating specific innate immunity signaling molecules and pathways. In particular, an immunomodulator composition is used to stimulate innate immunity signaling molecules and pathways.

### SUMMARY OF THE INVENTION

The present invention relates to methods of using immunostimulatory plasmids to modulate innate immunity signaling molecules and pathways. The immunostimulatory plasmid may comprise a nucleic acid sequence having at least 89% sequence identity with the sequence of SEQ ID NO: 1, SEQ ID NO: 2, SEQ ID NO: 3, SEQ ID NO: 4, or a combination thereof. In some aspects, the immunostimulatory plasmid may comprise a nucleic acid molecule having at least 84% sequence identity with the sequence of SEQ ID NO: 4. In some aspects, the immunostimulatory plasmid may comprise the sequence of SEQ ID NO: 1. In some aspects, the immunostimulatory plasmid may comprise the sequence of SEQ ID NO: 4. In some aspects, the immunostimulatory plasmid may comprise the sequence of SEQ ID NO: 2. In some aspects, the immunostimulatory plasmid may comprise the sequence of SEQ ID NO: 3.

In other aspects, the immunostimulatory plasmid may consist of a nucleic acid sequence having at least 89% sequence identity with the sequence of SEQ ID NO: 1, SEQ ID NO: 2, SEQ ID NO: 3, SEQ ID NO: 4 or a combination thereof. In some aspects, the immunostimulatory plasmid may consist of a nucleic acid molecule having at least 84% sequence identity with the sequence of SEQ ID NO: 4. In some aspects, the immunostimulatory plasmid may consist of the sequence of SEQ ID NO: 1. In some aspects, the immunostimulatory plasmid may consist of the sequence of SEQ ID NO: 4. In some aspects, the immunostimulatory plasmid may consist of the sequence of SEQ ID NO: 2. In some aspects, the immunostimulatory plasmid may consist of the sequence of SEQ ID NO: 3.

In some aspects, the immunostimulatory plasmid preferably does not comprise a nucleic acid sequence encoding a full-length or functional selectable or screenable marker. In other aspects, the immunostimulatory plasmid comprises a nucleic acid sequence encoding a selectable or screenable marker that is not an antibiotic resistance gene.

The present invention also relates to pharmaceutical formulations comprising any of the immunostimulatory plasmids, or DNA sequences, described herein and a pharmaceutically acceptable carrier.

The present invention further relates to immunomodulator compositions comprising a cationic liposome delivery vehicle and any of the immunostimulatory plasmids, or DNA sequences, described herein.

In some aspects, the present invention relates to methods of using the immunostimulatory plasmids, or DNA sequences, described herein. Suitable methods of use include therapeutic administration to a subject. Such therapeutic administration includes prophylactic treatment, metaphylactic treatment, and post-infection treatment of a subject or subjects.

The present invention relates to methods of stimulating or eliciting an immune response in a subject. In some aspects, the methods include stimulating an immune response in a subject by administering to the subject an immunomodulator composition described herein. In some aspects, the methods include stimulating an immune response in a subject by administering to the subject an immunostimulatory plasmid, or DNA sequence, described herein.

Methods are also provided for increasing weight gain of cattle diagnosed with bovine respiratory disease comprising administering an antimicrobial agent to the subject in combination with an immunomodulator composition comprising a nucleic acid sequence having at least 80% homology with SEQ ID NO: 1 and a lipid delivery vehicle, wherein the combination increases weight in the subject.

Also provided herein are methods for increasing weight gain of cattle diagnosed with bovine respiratory disease comprising administering an antimicrobial agent to the subject in combination with an immunomodulator composition comprising a nucleic acid sequence having at least 80% homology with SEQ ID NO:4 and a lipid delivery vehicle, wherein the combination increases weight gain in the subject.

Other objects and features will be in part apparent and in part pointed out hereinafter.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a map of the pMB75.6 plasmid (SEQ ID NO: 2);

FIG. 2 shows a map of the pGCMB75.6 plasmid (SEQ ID NO: 1);

FIG. 3 shows a map of the pLacZ75.6 plasmid (SEQ ID NO: 4);

FIG. 4 graphically illustrates IFN α1 (blue, diamond) activation of IRF-3 in comparison to control (red, square, PBS control);

FIG. 5 graphically illustrates the results of contacting IRF-THP-1 cells with immunomodulator compositions described herein or a positive control (INFα1). The immunomodulator compositions included SEQ ID NO. 2 DNA unformulated (Seq No 2), and SEQ ID NO. 2 DNA formulated (Seq No 2-F) with liposome carrier.

FIG. 6 graphically illustrates the results of contacting IRF-THP-1 cells, stably transfected with the IRF-3 reporter, with immunomodulator compositions described herein. The immunomodulator compositions included SEQ ID NO. 2 DNA unformulated (blue, diamond, Seq No 2), SEQ ID NO. 1 DNA unformulated (red, square, Seq No 1), SEQ ID NO. 2-Formulated (Seq No. 2-F, green, triangle), SEQ ID NO. 1 formulated (Seq No 1-F, purple, cross), and PBS (negative control, blue, star);

FIG. 7 graphically illustrates the results of contacting IRF-THP-1 cells with immunomodulator compositions (195 ng/mL) described herein and known standard ligand tools (250 ng/mL). The immunomodulator compositions included SEQ ID NO. 2 unformulated (Seq No 2), SEQ ID NO. 1 unformulated (Seq No 1), SEQ ID NO. 2-Formulated (Seq No 2-Form), SEQ ID NO. 1 formulated (Seq No 1-Form) and DOTIM/cholesterol (formulation alone), and PBS control (negative control). The known standard ligand tools included HSV-60-Lyovec; VACV-Lyovec; POLY-(dA/dT)-Lyovec;

FIG. 8 graphically illustrates the results of contacting IRF-THP-1 cells, with immunomodulator compositions (195 ng/mL) described herein and known standard ligand tools (1000 ng/mL). The immunomodulator compositions included SEQ ID NO. 2 unformulated (Seq No 2), SEQ ID NO. 1 unformulated (Seq No 1), SEQ ID NO. 2-Formulated (Seq No 2-Form), SEQ ID NO. 1 formulated (Seq NO 1-Form) and DOTIM (formulation alone), and PBS control (negative control). The known standard ligand tools included HSV-60-Lyovec; VACV-Lyovec; POLY-(dA/dT)-Lyovec;

FIG. 9 graphically illustrates the results of contacting IRF-THP-1 cells with known cytosolic DNA recognition activators (HSV-60; red, square; VACV 70; green, triangle; POLY, purple, cross; PBS negative control, blue, cross; Liposome, blue, diamond);

FIG. 10 graphically illustrates the results of contacting IRF-THP-1 cells with immunomodulator compositions described herein. The immunomodulator compositions included SEQ ID NO. 2 (Seq No 2, blue, diamond); SEQ ID NO. 1 (Seq No 1, red, square); SEQ ID NO. 2 plus liposome (Seq No 2-F, green, triangle); SEQ ID NO. 1 plus liposome (Seq No 1, purple, cross); and PBS negative control (blue, cross);

FIG. 11 shows the dose response curves of IFN-α1 (blue, diamond), SEQ ID NO. 2 unformulated (Seq No 2, red, square), SEQ ID NO. 2 formulated (Seq No 2-F, green, triangle), and PBS control (purple, cross) over a concentration range of 1.5-50 μg/mL in IRF-THP-1 cells measuring SEAP signal;

FIG. 12 shows the dose response curves of SEQ ID NO. 2 unformulated (Seq No 2, blue diamond), SEQ ID NO. 1 unformulated (Seq No 1, red, square), SEQ ID NO. 2 formulated (Seq No 2-F, green, triangle), SEQ ID NO. 1 formulated (Seq No 1-F, purple, cross), and PBS control (black, square) over a concentration range of 0.3-25 μg/mL in IRF-THP-1 cells measuring SEAP signal;

FIG. 13 graphically illustrates stimulation of IRF-THP-1 cells contacted with SEQ ID NO. 2 unformulated (Seq No 2), SEQ ID NO. 1 unformulated (Seq No 1), SEQ ID NO. 2-Formulated (Seq No 2-F), SEQ ID NO. 1 formulated (Seq No 1-F) and Liposome/formulation alone, PBS control (negative control), and known standard ligand tools including HSV-60-Lyovec; VACV-Lyovec; and Poly-(dA/dT)-Lyovec;

FIG. 14 graphically illustrates stimulation of IRF-THP-1 cells contacted with SEQ ID NO. 2 unformulated (Seq No 2), SEQ ID NO. 1 unformulated (Seq No 1), SEQ ID NO 2-Formulated (Seq No 2-Form), SEQ ID NO. 1 formulated (Seq No 1-Form) and Liposome/formulation alone, PBS control (negative control), and known standard ligand tools including HSV-60-Lyovec; VACV-Lyovec; and POLY-(dA/dT)-Lyovec;

FIG. 15 graphically illustrates stimulation of IRF-THP-1 cells contacted with SEQ ID NO. 2 unformulated (Seq No 2), SEQ ID NO. 1 unformulated (Seq No 1), SEQ ID NO. 2-Formulated (Seq No 2-Form), SEQ ID NO. 1 formulated (Seq No 1-Form), PBS control (negative control), known standard ligand tools including HSV-60-Lyovec; VACV-Lyovec; and POLY-(dA/dT)-Lyovec, Lyovec only, and SEQ ID NO. 2 formulated with LyoVec, SEQ ID NO. 1 formulated with LyoVec, and IFN-α1;

FIG. 16 shows dose response curves of SEQ ID NO. 2 and SEQ ID NO. 1 in IRF-THP-1 cells as unformulated (SEQ ID NO. 2-naked, green triangle; and SEQ ID NO. 1 naked, orange circle), liposome-formulated (Seq No 2-F, blue diamond; and Seq No 1-F, purple cross), and as LyoVec-formulated (Seq No 2-LyoVec, red square; and Seq No 1 LyoVec, blue star);

FIG. 17 shows dose response curves of SEQ ID NO. 2 and SEQ ID NO. 1 in IRF-THP-1 cells as formulated with LyoVec transfection agent including Seq No 2/LyoVec (blue, diamond), Seq No 1/LyoVec (red, square), LyoVec only (green, triangle), blank (blue, star), and IFNα1 (orange, circle);

FIG. 18 shows dose response curves of SEQ ID NO. 2 and SEQ ID NO. 1 in IRF-THP-1 cells as formulated with Mirus transfection agent including SEQ ID NO. 2/Mirus (Seq No 2, blue, diamond), SEQ ID NO. 1/Mirus (Seq No 1, red, square), Mirus only (green, triangle), SEQ ID NO. 2 unformulated (Seq No 2, purple, cross), blank (blue, star), and IFNα1 (orange, circle);

FIG. 19 shows dose response curves of SEQ ID NO. 2 and SEQ ID NO. 1 in IRF-THP-1 cells as formulated with X-tremeGen transfection agent including SEQ ID NO. 2/X-tremeGen (Seq No 2/XtremeGen, blue, diamond), SEQ ID NO. 1/X-tremeGen (Seq No 1/xtremeGen, red, square), X-tremeGen only (green, triangle), SEQ ID NO. 1 unformulated (Seq No 1, purple, cross), blank (blue, star), and IFNα1 (orange, circle);

FIG. 20 shows the dose-response of B16 Blue™ ISG cells after stimulation with IFNα1 positive control;

FIG. 21 graphically illustrates the stimulation of B16-Blue™ ISG cells contacted with PBS control, IFNα1, SEQ ID NO. 2-formulated (Seq No 2-Form), SEQ ID NO. 1 formulated (Seq No 1-Form), SEQ ID NO. 2 unformulated (Seq No 2), SEQ ID NO. 1 unformulated (Seq No 1), Liposome/formulation alone (Liposome control), 3′-3′-cGAMP, and POLY-(dA/dT);

FIG. 22 graphically illustrates the stimulation of THP-1-Blue™ ISG cells contacted with PBS control, IFNα1, SEQ ID NO. 2-formulated (Seq No 2-Form), SEQ ID NO. 1 formulated (Seq No 1-Form), SEQ ID NO. 2 unformulated (Seq No 2), SEQ ID NO. 1 unformulated (Seq No 1), and Liposome/formulation alone (Liposome control);

FIG. 23 graphically illustrates the stimulation of THP-1-Blue™ ISG-KD-STING cells contacted with PBS control, IFNα1, SEQ ID NO. 2-formulated (Seq No 2-F), SEQ ID NO. 1 formulated (Seq No 1-F), SEQ ID NO. 2 unformulated (Seq No 2), SEQ ID NO. 1 unformulated (Seq No 1), and Liposome/formulation alone (Liposome control);

FIG. 24A and FIG. 24B graphically illustrate cytosolic DNA recognition of SEQ ID NO. 2 formulated (Plasmid-F);

FIG. 25A and FIG. 25B graphically illustrate the central role of STING in immunomodulating function of SEQ ID NO. 2 formulated (Plasmid-F);

FIG. 26A and FIG. 26B graphically illustrate the central role of STING in immunomodulating function caused by SEQ ID NO. 2 formulated (Seq No 2-F) and SEQ ID NO. 1 formulated (Seq No 1-F);

FIG. 27A, FIG. 27B, and FIG. 27C illustrate the ability of SEQ ID NO. 2 formulated (Seq No 2-F) to induce interferon release in porcine peripheral blood mononuclear cells;

FIG. 28A, FIG. 28B, and FIG. 28C illustrate the ability of SEQ ID NO. 2 formulated (Seq No 2-F) to induce interferon release in bovine peripheral blood mononuclear cells;

FIG. 29 graphically illustrates measured rectal temperatures during the course of testing;

FIG. 30A and FIG. 30B graphically illustrate mean body weight at the start and end of the animal phase;

FIG. 31A, FIG. 31B, FIG. 31C, FIG. 31D, FIG. 31E, and FIG. 31F depict hematological data gathered from porcine subjects;

FIG. 32A, FIG. 32B, FIG. 32C, FIG. 32D, FIG. 32E, and FIG. 32F depict hematological data gathered from porcine subjects;

FIG. 33A, FIG. 33B, FIG. 33C, FIG. 33D, FIG. 33E, FIG. 33F, FIG. 33G, and FIG. 33H depict hematological data gathered from porcine subjects;

FIG. 34A, FIG. 34B, FIG. 34C and FIG. 34D depict the content and relative change of serum cytokine content of IL 1 and IL 2 before and after treatments;

FIG. 35A, FIG. 35B, FIG. 35C, and FIG. 35D graphically illustrate content and relative change of serum cytokine content of IL 4 and IL 6 before and after treatments;

FIG. 36A, FIG. 36B, FIG. 36C, and FIG. 36D illustrate content and relative change of serum cytokine content of IL 8 and IL 10 before and after treatments;

FIG. 37A, FIG. 37B, FIG. 37C, and FIG. 37D illustrate content and relative change of serum cytokine content of IL 12 and INFg before and after treatments;

FIG. 38A and FIG. 38B illustrate the content and relative change of serum cytokine content of TNFa before and after treatments;

FIG. 39A, FIG. 39B, FIG. 39C, FIG. 39D, FIG. 39E, and FIG. 39F illustrate content and relative change of serum cytokine content of IL 1, IL2 and IL 4 before and after intravenous administration of low or high dose of test substance;

FIG. 40A, FIG. 40B, FIG. 40C, FIG. 40D, FIG. 40E, and FIG. 40F illustrate content and relative change of serum cytokine content of IL 6, IL 8 and IL 10 before and after intravenous administration of low or high dose of test substance;

FIG. 41A, FIG. 41B, FIG. 41C, FIG. 41D, FIG. 41E, FIG. 41F, FIG. 41G, and FIG. 41H illustrate relative amount and proportional change of IL 1 mRNA after treatment (i.m. and s.c. inoculation with high or low doses of test substance; amounts are determined at time points −1, 2, 6, 24 and 48 hours post inoculation).;

FIG. 42A, FIG. 42B, FIG. 42C, and FIG. 42D illustrate relative amount and proportional change of IL 2 mRNA after treatment (i.m. and s.c. inoculation with high or low doses of test substance; amounts are determined at time points −1, 2, 6, 24 and 48 hours post inoculation);

FIG. 43A, FIG. 43B, FIG. 43C, and FIG. 43D illustrate relative amount and proportional change of IL 2 mRNA after treatment (i.m. and s.c. inoculation with high or low doses of test substance; amounts are determined at time points −1, 2, 6, 24 and 48 hours post inoculation);

FIG. 44A, FIG. 44B, FIG. 44C, FIG. 44D, FIG. 44E, FIG. 44F, FIG. 44G, and FIG. 44H illustrate relative amount and proportional change of IL 6 mRNA after treatment (i.m. and s.c. inoculation with high or low doses of test substance; amounts are determined at time points −1, 2, 6, 24 and 48 hours post inoculation);

FIG. 45A, FIG. 45B, FIG. 45C, FIG. 45D, FIG. 45E, FIG. 45F, FIG. 45G, and FIG. 45H graphically illustrate relative amount and proportional change of IL 10 mRNA after treatment (i.m. and s.c. inoculation with high or low doses of test substance; amounts are determined at time points −1, 2, 6, 24 and 48 hours post inoculation);

FIG. 46A, FIG. 46B, FIG. 46C, FIG. 46D, FIG. 46E, FIG. 46F, FIG. 46G, and FIG. 46H relative amount and proportional change of IL 12 mRNA after treatment (i.m. and s.c. inoculation with high or low doses of test substance; amounts are determined at time points −1, 2, 6, 24 and 48 hours post inoculation);

FIG. 47A, FIG. 47B, FIG. 47C, FIG. 47D and FIG. 47E illustrate the proportional change of different cytokines after administration of a high dose of the test substance (i.m. administration and s.c. administration route are combined; amounts are determined at time points −1, 2, 6, 24 and 48 hours post inoculation);

FIG. 48A, FIG. 48B, FIG. 48C, FIG. 48D and FIG. 48E illustrate the proportional change of different cytokines after administration of a high dose of the test substance (i.m. administration and s.c. administration route are combined; amounts are determined at time points −1, 2, 6, 24 and 48 hours post inoculation);

FIG. 49A and FIG. 49B illustrate change of cytokine mRNA expression observed in one pig after intravenous administration of a high (upper panel) or low (lower panel) dose of the test substance; and,

FIG. 50A and FIG. 50B show the percentage of lung lesions (FIG. 50 A) and mortality (FIG. 50B) due to BRD in steers receiving treatment with Seq No 2-F and controls.

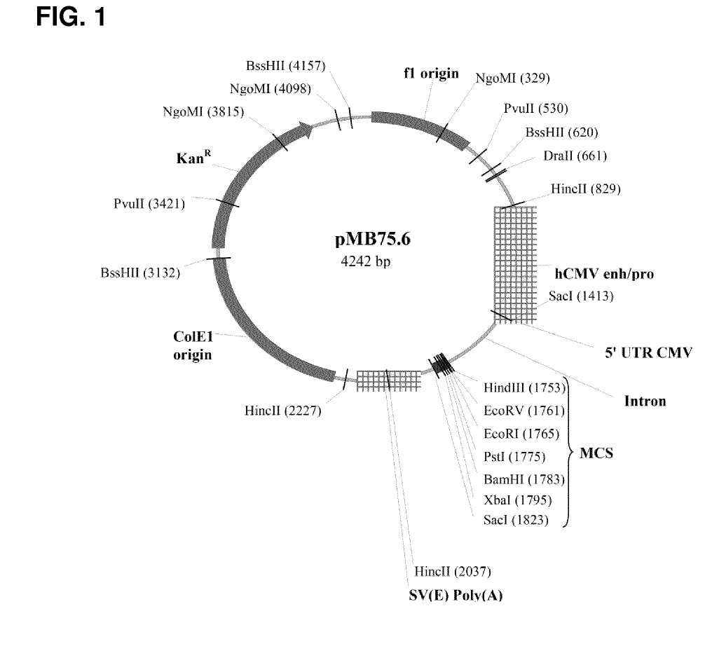

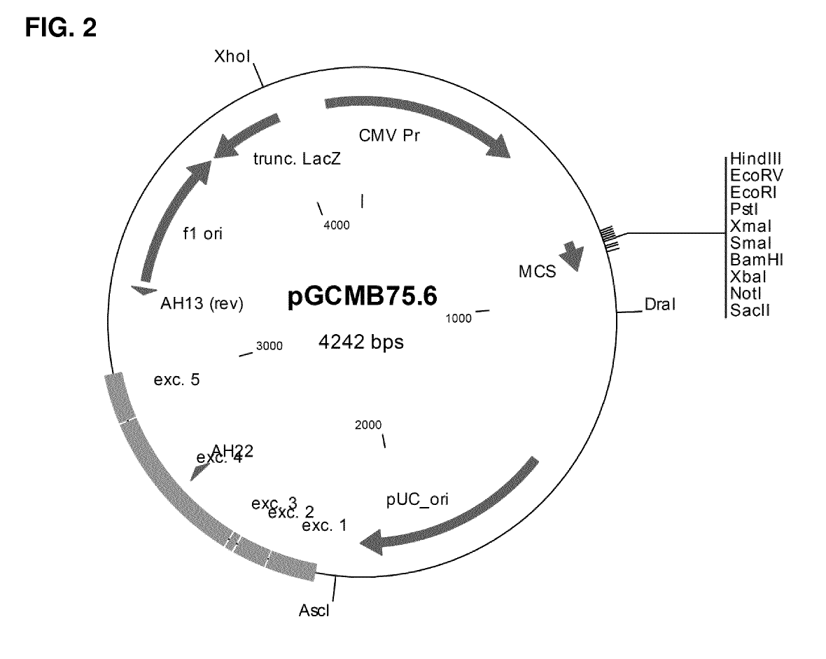

### DETAILED DESCRIPTION OF THE INVENTION

In accordance with the present invention, a composition capable of activating cytosolic DNA surveillance molecules in a recipient subject, as well as methods of use, have been discovered. In particular, the present invention relates to novel nucleic acid compositions, or immunomodulator compositions, and uses thereof. It has been discovered that such immunomodulator compositions be used to modulate the immune system of a subject. The invention is particularly useful in the treatment and prevention of infectious diseases caused by microorganisms, such as, without limitation, viruses, bacteria, mold, fungus, yeast, parasites and other microbes known in the art. The compositions and methods of using the immunomodulator compositions are discussed in more detail below.

### I. Compositions

Compositions useful in this invention, such as those described herein, are generally able to be used as a prophylactic therapy, metaphylactic therapy, or treatment therapy for infectious diseases. Such compositions are referred to herein as immunomodulator compositions. The immunomodulator compositions include at least an immunostimulatory plasmid or immunostimulatory DNA sequence, capable of activating cytosolic DNA surveillance molecules in a recipient subject. In some aspects, the immunomodulator compositions may also include a liposome delivery vehicle.

### A. Nucleic Acids

In some aspects the present invention relates to nucleic acid molecules useful for the treatment or prevention of infectious disease causing agents. The nucleic acid molecules described herein may be included in an immunostimulatory plasmid, as linear double stranded or single stranded DNA, amino acid sequence, ribonucleic acid (RNA), or combinations thereof. In some aspects, the present invention relates to nucleic acid molecules, vectors, and host cells (in vitro, in vivo, or ex vivo) which contain the immunostimulatory plasmid or immunostimulatory DNA sequence.

The nucleic acid molecules described herein are enriched in CpG motifs. Such CpG motifs may induce immune stimulation via specific Toll-like receptors, such as TLR9 and TLR21. In addition the nucleic acid molecules described herein also contain non-CpG immunostimulatory motifs. In some aspects, the nucleic acid molecules contain about 2-20% CpG motifs over the frequency of CpG motifs expected in random nucleic acid sequences. In some aspects, the nucleic acid molecules contain about 3, 4, 5, 6, 7, 8, 9, 10, 11, 12, 13, 14, 15, 16, 17, 18, 19, 20, 25, 30, 35, 40%, or more CpG motifs over the frequency of CpG motifs expected in random nucleic acid sequences. In some aspects, the nucleic acid molecules contain about 10% CpG motifs over the frequency of CpG motifs expected in random nucleic acid sequences. In some aspects, compared to vertebrate DNA, an enrichment of CpG motifs of more than 10-fold is observed. In some aspects, the nucleic acid molecules contain about 2 to 50 fold, or more CpG motifs compared to vertebrate DNA. In some aspects, the nucleic acid molecules contain about 3, 4, 5, 6, 7, 8, 9, 10, 11, 12, 13, 14, 15, 16, 17, 18, 19, 20, 25, 30, 35, 40, 45, 50, 55 fold or more CpG motifs compared to vertebrate DNA.

In some aspects, the present invention relates to immunostimulatory plasmids, or DNA sequences, that do not comprise an antibiotic resistance gene. The plasmids may be devoid of any selectable or screenable marker genes. For example, the pGCMB75.6 plasmid described herein does not comprise any full-length or functional selectable or screenable marker genes. The sequence of pGCMB75.6 is provided in SEQ ID NO: 1.

In some aspects, the immunostimulatory plasmids described herein preferably do not comprise a nucleic acid sequence coding for a full-length or functional selectable or screenable marker. In some aspects, the immunostimulatory plasmids do not comprise an antibiotic resistance gene. For example, the plasmids do not comprise a kanamycin resistance gene. In some aspects, the plasmids described herein preferably do not encode an immunogen.

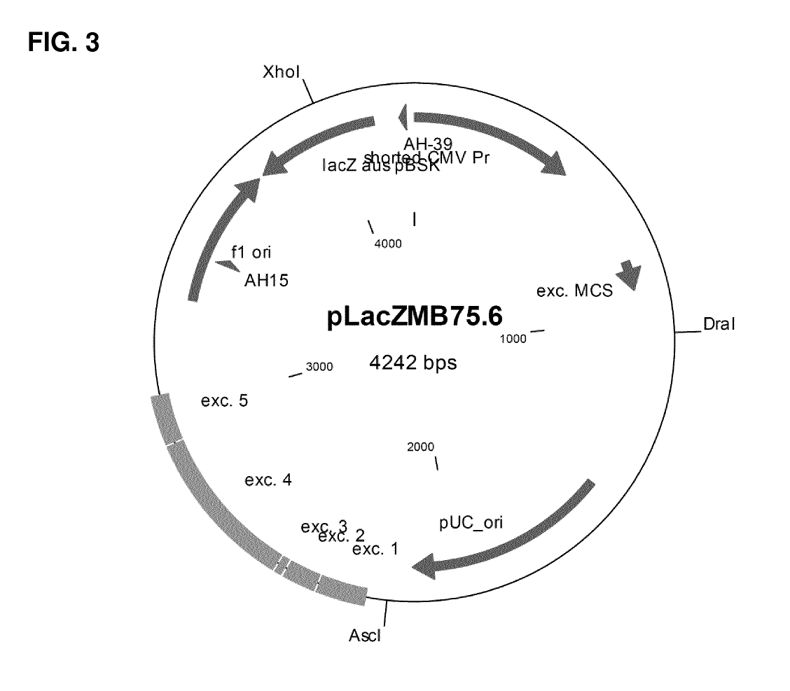

In some aspects, the immunostimulatory plasmids may comprise a nucleic acid sequence coding for a selectable or screenable marker gene that is not an antibiotic resistance gene. For example, the pLacZMB75.6 plasmid described herein comprises a LacZ gene as a screenable marker. A map of pLacZMB75.6 is provided in FIG. 3 and the nucleotide sequence of pLacZMB75.6 is provided as SEQ ID NO: 4. As shown in FIG. 3, pLacZMB75.6 is similar to pGCMB75.6, but contains a LacZ screenable marker.

It will be appreciated that the nucleotide sequences of the pMB75.6, pGCMB75.6 or pLacZMB75.6 plasmids may be varied to a certain extent without significantly adversely affecting their immunostimulatory properties. In some aspects, the present invention relates to an immunostimulatory plasmid comprising a nucleic acid sequence having at least 89% sequence identity with the sequence of pGCMB75.6 (SEQ ID NO: 1). The immunostimulatory plasmid preferably comprises a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of pGCMB75.6 (SEQ ID NO: 1). In some aspects, the immunostimulatory plasmid more preferably comprises the sequence of pGCMB75.6 (SEQ ID NO: 1).

In some aspects, the present invention relates to an immunostimulatory plasmid comprising a nucleic acid sequence having at least 84% sequence identity with the sequence of pLacZMB75.6 (SEQ ID NO: 4). The immunostimulatory plasmid preferably comprises a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of pLacZMB75.6 (SEQ ID NO: 4). In some aspects, the immunostimulatory plasmid more preferably comprises the sequence of pLacZMB75.6 (SEQ ID NO: 4).

In some aspects, the present invention relates to an immunostimulatory plasmid comprising a nucleic acid sequence having at least 80% sequence identity with the sequence of SEQ ID NO: 2. The immunostimulatory plasmid preferably comprises a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of SEQ ID NO: 2. In some aspects, the immunostimulatory plasmid more preferably comprises the sequence of SEQ ID NO: 2.

In some aspects, the present invention relates to an immunostimulatory plasmid comprising a nucleic acid sequence having at least 80% sequence identity with the sequence of SEQ ID NO: 3. The immunostimulatory plasmid preferably comprises a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of SEQ ID NO: 3. In some aspects, the immunostimulatory plasmid more preferably comprises the sequence of SEQ ID NO: 3.

In some aspects, the present invention relates to an immunostimulatory plasmid consisting of a nucleic acid sequence having at least 89% sequence identity with the sequence of pGCMB75.6 (SEQ ID NO: 1). The immunostimulatory plasmid preferably consists of a nucleic acid sequence having at least 75%, at least 76%, at least 77%, at least 78%, at least 79%, at least 80%, at least 81%, at least 82%, at least 83%, at least 84%, at least, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of pGCMB75.6 (SEQ ID NO: 1). In some aspects, the immunostimulatory plasmid more preferably consists of the sequence of pGCMB75.6 (SEQ ID NO: 1).

In some aspects, the present invention relates to an immunostimulatory plasmid consisting of a nucleic acid sequence having at least 84% sequence identity with the sequence of pLacZMB75.6 (SEQ ID NO: 4). The immunostimulatory plasmid preferably consists of a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of pLacZMB75.6 (SEQ ID NO: 4). In some aspects, the immunostimulatory plasmid more preferably consists of the sequence of pLacZMB75.6 (SEQ ID NO: 4).

In some aspects, the present invention relates to an immunostimulatory plasmid consisting of a nucleic acid sequence having at least 80% sequence identity with the sequence of SEQ ID NO: 2. The immunostimulatory plasmid preferably consists of a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of SEQ ID NO: 2. In some aspects, the immunostimulatory plasmid more preferably consists of the sequence of SEQ ID NO: 2.

In some aspects, the present invention relates to an immunostimulatory plasmid consisting of a nucleic acid sequence having at least 80% sequence identity with the sequence of SEQ ID NO: 3. The immunostimulatory plasmid preferably consists of a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of SEQ ID NO: 3. In some aspects, the immunostimulatory plasmid more preferably consists of the sequence of SEQ ID NO: 3.

Another important aspect of this invention provides for immunostimulatory DNA sequences or immunostimulatory plasmids capable of stimulating an immune response including nucleic acid sequences that hybridize under high stringency conditions to SEQ ID NO: 1, SEQ ID NO: 2, SEQ ID NO: 3, or SEQ ID NO: 4. Suitable nucleic acid sequences include those that are homologous, substantially similar, or identical to the nucleic acids of the present invention. In some aspects, homologous nucleic acid sequences will have a sequence similarity of at least about 75%, 76%, 77%, 78%, 79%, 80% 81%, 82%, 83%, 84%, 85%, 86%, 87%, 88%, 89%, 90%, 91%, 92%, 93%, 94%, 95%, 96%, 97%, 98%, 99%, or 100% to SEQ ID NO: 1 or the respective complementary sequence. In other aspects, homologous nucleic acid sequences will have a sequence similarity of at least about 75%, 76%, 77%, 78%, 79%, 80% 81%, 82%, 83%, 84%, 85%, 86%, 88%, 89%, 90%, 91%, 92%, 93%, 94%, 95%, 96%, 97%, 98%, 99%, or 100% to SEQ ID NO: 4 or the respective complementary sequence. In other aspects, homologous nucleic acid sequences will have a sequence similarity of at least about 75%, 76%, 77%, 78%, 79%, 80% 81%, 82%, 83%, 84%, 85%, 86%, 88%, 89%, 90%, 91%, 92%, 93%, 94%, 95%, 96%, 97%, 98%, 99%, or 100% to SEQ ID NO: 2 or the respective complementary sequence. In other aspects, homologous nucleic acid sequences will have a sequence similarity of at least about 75%, 76%, 77%, 78%, 79%, 80% 81%, 82%, 83%, 84%, 85%, 86%, 88%, 89%, 90%, 91%, 92%, 93%, 94%, 95%, 96%, 97%, 98%, 99%, or 100% to SEQ ID NO: 3 or the respective complementary sequence. Sequence similarity may be calculated using a number of algorithms known in the art, such as BLAST, described in Altschul, S. F., et al., J. Mol. Biol. 215:403-10, 1990. The nucleic acids may differ in sequence from the above-described nucleic acids due to the degeneracy of the genetic code. In general, a reference sequence will be 18 nucleotides, more usually 30 or more nucleotides, and may comprise the entire nucleic acid sequence of the composition for comparison purposes.

Nucleotide sequences that can hybridize to SEQ ID NO: 1, SEQ ID NO: 2, SEQ ID NO: 3, or SEQ ID NO: 4 are contemplated herein. Stringent hybridization conditions include conditions such as hybridization at 50° C. or higher and 0.1×SSC (15 mM sodium chloride/1.5 mM sodium citrate). Another example is overnight incubation at 42° C. in a solution of 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA, followed by washing in 0.1×SSC at about 65° C. Exemplary stringent hybridization conditions are hybridization conditions that are at least about 80%, 85%, 90%, or 95% as stringent as the above specific conditions. Other stringent hybridization conditions are known in the art and may also be employed to identify homologs of the nucleic acids of the invention (Current Protocols in Molecular Biology, Unit 6, pub. John Wiley & Sons, N.Y. 1989).

Mutant nucleotides of the DNA molecules described herein may be used, so long as mutants include nucleic acid sequences maintain the ability to activate cytosolic DNA surveillance molecules as described herein. The DNA sequence of such a mutation will usually differ by one or more nucleotides or amino acids. The sequence changes may be substitutions, insertions, deletions, or a combination thereof. Techniques for mutagenesis of cloned genes are known in the art. Methods for site specific mutagenesis may be found in Gustin et al., Biotechniques 14:22, 1993; Barany, Gene 37:111-23, 1985; Colicelli et al., Mol. Gen. Genet. 199:537-9, 1985; and Sambrook et al., Molecular Cloning: A Laboratory Manual, CSH Press 1989, pp. 15.3-15.108 and all incorporated herein by reference. In summary, the invention relates to nucleic acid sequences capable of activating cytosolic DNA surveillance molecules in a subject and variants or mutants thereof. Also, the invention encompasses the intermediatary RNAs encoded by the described nucleic acid sequences, as well as any resultant amino acid sequences encoded.

In some aspects, where the nucleotide sequence of the immunostimulatory plasmid varies from the sequences provided in SEQ ID NOs. 1, 2, 3, and 4 the CpG dinucleotides in the plasmid are preferably left intact. Alternatively, if the nucleotide sequence of the plasmid is altered such that a CpG dinucleotide is eliminated, the sequence of the plasmid may be altered at another location such that the total number of CpG dinucleotides in the plasmid remains the same. Further CpG dinucleotides in addition to those already present in the nucleotide sequences of pGCMB75.6 or pLacZMB75.6 may also be introduced into the plasmid. Thus, for example, the immunostimulatory plasmids described herein preferably comprise at least about 200, at least about 220, at least about 240, at least about 260, at least about 270, at least about 275, at least about 280, at least about 283, at least about 285, or at least about 288 CpG dinucleotides. For example, the immunostimulatory plasmid can comprise 283 CpG dinucleotides.

In some aspects, where the nucleotide sequence of the immunostimulatory plasmid varies from the sequences provided herein, the CpG motif types in the plasmid are varied to modulate the resultant activation of the cytosolic DNA surveillance molecules. For example, the number of immune stimulatory CpG motifs may be increased to increase the activation of specific cytosolic DNA surveillance molecules responsive to a specific threshold of immunostimulatory plasmid/DNA. By way of further example, the number of non-immune stimulatory CpG motifs may be increased to decrease the activation of specific cytosolic DNA surveillance molecules and/or increase activation of other DNA surveillance molecules.

In particular, the present invention relates to pharmaceutical formulations comprising any of the immunostimulatory plasmids or DNA sequences described herein and a pharmaceutically acceptable carrier.

### B. Immunomodulator

Suitable immunomodulator compositions for use with the immunostimulatory plasmids described herein are described in U.S. Patent Application Publications Nos. 2012/0064151 A1 and 2013/0295167 A1 the contents of both of which are hereby incorporated by reference in their entirety.

The immunomodulator composition comprises a liposome delivery vehicle and at least one of the immunostimulatory plasmids, or DNA sequences, described herein.

A suitable liposome delivery vehicle comprises a lipid composition that is capable of delivering nucleic acid molecules to the tissues of the treated subject. A liposome delivery vehicle is preferably capable of remaining stable in a subject for a sufficient amount of time to deliver a nucleic acid molecule and/or a biological agent. For example, the liposome delivery vehicle is stable in the recipient subject for at least about five minutes, for at least about 1 hour, or for at least about 24 hours.

A liposome delivery vehicle of the present invention comprises a lipid composition that is capable of facilitating the delivery of a nucleic acid molecule into a cell. When the nucleic acid molecule encodes one or more proteins, the nucleic acid:liposome complex preferably has a transfection efficiency of at least about 1 picogram (pg) of protein expressed per milligram (mg) of total tissue protein per microgram (μg) of nucleic acid delivered. For example, the transfection efficiency of a nucleic acid: liposome complex can be at least about 10 pg of protein expressed per mg of total tissue protein per μg of nucleic acid delivered; or at least about 50 pg of protein expressed per mg of total tissue protein per μg of nucleic acid delivered. The transfection efficiency of the complex may be as low as 1 femtogram (fg) of protein expressed per mg of total tissue protein per μg of nucleic acid delivered, with the above amounts being more preferred.

A preferred liposome delivery vehicle of the present invention is between about 100 and 500 nanometers (nm) in diameter. For example, the liposome delivery vehicle can be between about 150 and 450 nm or between about 200 and 400 nm in diameter.

Suitable liposomes include any liposome, such as those commonly used in, for example, gene delivery methods known to those of skill in the art. Preferred liposome delivery vehicles comprise multilamellar vesicle (MLV) lipids and extruded lipids. Methods for preparation of MLVs are well known in the art. More preferred liposome delivery vehicles comprise liposomes having a polycationic lipid composition (i.e., cationic liposomes) and/or liposomes having a cholesterol backbone conjugated to polyethylene glycol. Exemplary cationic liposome compositions include, but are not limited to, N-[1-(2,3-dioleyloxy)propyl]-N,N,N-trimethylammonium chloride (DOTMA) and cholesterol, N-[1-(2,3-dioleoyloxy)propyl]-N,N,N-trimethylammonium chloride (DOTAP) and cholesterol, 1-[2-(oleoyloxy)ethyl]-2-oleyl-3-(2-hydroxyethyl)-imidazolinium chloride (DOTIM) and cholesterol, dimethyldioctadecylammonium bromide (DDAB) and cholesterol, and combinations thereof. A most preferred liposome composition for use as a delivery vehicle includes DOTIM and cholesterol.

A suitable nucleic acid molecule includes any of the immunostimulatory plasmids described herein. Coding nucleic acid sequences encode at least a portion of a protein or peptide, while non-coding sequence does not encode any portion of a protein or peptide. According to the present invention, “non-coding” nucleic acids can include regulatory regions of a transcription unit, such as a promoter region. The term, “empty vector” can be used interchangeably with the term “non-coding,” and particularly refers to a nucleic acid sequence in the absence of a protein coding portion, such as a plasmid vector without a gene insert. Expression of a protein encoded by the plasmids described herein is not required for activation of cytosolic DNA surveillance molecules; therefore the plasmids need not contain any coding sequences operatively linked to a transcription control sequence. However, further advantages may be obtained (i.e., antigen-specific and enhanced immunity) by including in the composition nucleic acid sequence (DNA or RNA) which encodes an immunogen and/or a cytokine. Such a nucleic acid sequence encoding an immunogen and/or a cytokine may be included in the immunostimulatory plasmids described herein, or can be included in a separate nucleic acid (e.g., a separate plasmid) in the composition.

Complexing a liposome with the immunostimulatory plasmids described herein may be achieved using methods standard in the art or as described in U.S. Pat. No. 6,693,086, the contents of which are hereby incorporated by reference in their entirety. A suitable concentration of a plasmid to add to a liposome includes a concentration effective for delivering a sufficient amount of the plasmid into a subject such that a systemic immune response is elicited. For example, from about 0.1 μg to about 10 μg of plasmid can be combined with about 8 nmol liposomes, from about 0.5 μg to about 5 μg of plasmid can be combined with about 8 nmol liposomes, or about 1.0 μg of plasmid can be combined with about 8 nmol liposomes. The ratio of plasmid to lipid (μg plasmid:nmol lipid) in a composition can be at least about 1:1 plasmid:lipid (e.g., 1 μg plasmid: 1 nmol lipid). For example, the ratio of plasmid to lipids can be at least about 1:5, at least about 1:10, or at least about 1:20. Ratios expressed herein are based on the amount of cationic lipid in the composition, and not on the total amount of lipid in the composition. The ratio of plasmid to lipids in a composition of the invention is suitably from about 1:1 to about 1:80 plasmid:lipid by weight; from about 1:2 to about 1:40 plasmid:lipid by weight; from about 1:3 to about 1:30 plasmid:lipid by weight; or from about 1:6 to about 1:15 plasmid:lipid by weight.

### C. Biological Agent

Any of the immunomodulator compositions described herein can further comprise at least one biological agent, in addition to the liposome delivery vehicle and at least one of the plasmids described herein.

Suitable biological agents are agents that are effective in preventing or treating diseases. Such biological agents include immune enhancer proteins, immunogens, vaccines, antimicrobials or any combination thereof. Suitable immune enhancer proteins are those proteins known to enhance immunity. By way of a non-limiting example, a cytokine, which includes a family of proteins, is a known immunity enhancing protein family. Suitable immunogens are proteins which elicit a humoral and/or cellular immune response such that administration of the immunogen to a subject mounts an immunogen-specific immune response against the same or similar proteins that are encountered within the tissues of the subject. An immunogen may include a pathogenic antigen expressed by a bacterium, a virus, a parasite or a fungus. Preferred antigens include antigens derived from organisms which cause an infectious disease in a subject. According to the present invention, an immunogen may be any portion of a protein, naturally occurring or synthetically derived, which elicits a humoral and/or cellular immune response. As such, the size of an antigen or immunogen may be as small as about 5-12 amino acids and as large as a full length protein, including any sizes in between. The antigen may be a multimer protein or fusion protein. The antigen may be a purified antigen. Alternatively, the immune enhancer protein or immunogen can be encoded by the immunostimulatory plasmid or by another nucleic acid included in the immunomodulator composition. Where the immune enhancer protein or immunogen is encoded by a nucleic acid molecule in the immunomodulator composition, the nucleic acid sequence encoding the immune enhancer protein or immunogen is operatively linked to a transcription control sequence, such that the immunogen is expressed in a tissue of a subject, thereby eliciting an immunogen-specific immune response in the subject, in addition to the non-specific immune response. Techniques to screen for immunogenicity, such as pathogen antigen immunogenicity or cytokine activity are known to those of skill in the art and include a variety of in vitro and in vivo assays.

Where the biological agent is a vaccine, the vaccine may include a live, infectious, viral, bacterial, or parasite vaccine or a killed, inactivated, viral, bacterial, or parasite vaccine. One or more vaccines, live or killed viral vaccines, may be used in combination with the immunomodulator composition of the present invention. Suitable vaccines include those known in the art for avian or bovine species.

The biological agent can be an antimicrobial. Suitable antimicrobials include: quinolones, preferably fluoroquinolones, β-lactams, and macrolide-lincosamide-streptogramin (MLS) antibiotics.

Suitable quinolones include benofloxacin, binfloxacin, cinoxacin, ciprofloxacin, clinafloxacin, danofloxacin, difloxacin, enoxacin, enrofloxacin, fleroxacin, gemifloxacin, ibafloxacin, levofloxacin, lomefloxacin, marbofloxacin, moxifloxacin, norfloxacin, ofloxacin, orbifloxacin, pazufloxacin, pradofloxacin, perfloxacin, sarafloxacin, sparfloxacin, temafloxacin, and tosufloxacin. Preferred fluoroquinolones include ciprofloxacin, danofloxacin, enrofloxacin, moxifloxacin, and pradofloxacin. Suitable naphthyridones include nalidixic acid.

Suitable β-lactams include penicillins (e.g., amoxicillin, ampicillin, azlocillin, benzathine penicillin, benzylpenicillin, carbenicillin, cloxacillin, co-amoxiclav [i.e. amoxicillin/clavulanic acid], dicloxacillin, flucloxacillin, methicillin, mezlocillin, nafcillin, oxacillin, phenoxymethylpenicillin, piperacillin, procaine penicillin, temocillin, and ticarcillin); cephalosporins (e.g., cefaclor, cefalonium, cefamandole, cefapririn, cefazolin, cefepime, cefixime, cefotaxime, cefoxitin, cefpirome, cefpodoxime, cefquinome, ceftazidime, ceftiofur, ceftriaxone, cefuroxime, cephalexin, cephalothin, and defotetan); carbapenems and penems (e.g., doripenem, ertapenem, faropenem, imipenem, and meropenem); monobactams (e.g., aztreonam, nocardicin A, tabtoxinine-β-lactam, and tigemonam); and β-lactamase inhibitors (e.g., clavulanic acid, sulbactam, and tazobactam). Preferred β-lactams include cephalosporins, in particular, cefazolin.

Suitable MLS antibiotics include clindamycin, lincomycin, pirlimycin, and any macrolide antibiotic. A preferred lincosamide antibiotic is pirlimycin.

Other antimicrobials include aminoglycosides, clopidol, dimetridazoles, erythromycin, framycetin, furazolidone, halofuginone, 2-pyridones, robenidine, sulfonamides, tetracyclines, trimethoprim, various pleuromutilins (e.g., tiamulin and valnemulin), and various streptomycin (e.g., monensin, narasin, and salinomycin).

Bovine respiratory disease, or bovine respiratory diseases complex, (BRD) is a leading cause of economic loss in the cattle industry. When the cattle are subjected to stressors, their innate and acquired immune functions are compromised which allows microorganisms that are part of the normal flora of bovine respiratory tracts to flourish and colonize the lower respiratory tract. The lower respiratory system is typically a sterile field, thus microbial proliferation can cause severe sickness and even death.

Combination therapies, in which the immunomodulator compositions of the present invention and administered in addition to an antimicrobial effective against BRD may be effected to stimulate an immune response as well as directly act on the pathogen. The combination therapy can decrease recovery times or even prevent infectious if administered prophylactically. A decrease in morbidity can result in increased productivity of feedlot animals. “Productivity” as used herein refers to the activities undertaken by a feedlot animal that results in weight gain. “Weight gain,” as used herein, may refer to an increase in average daily gain and/or average weight per animal. While sick and distressed animals may gain weight, the weight gain observed in animals receiving a combination therapy may outpace that of the sick animals. Therefore, some embodiments of the present invention provide for methods for increasing weight in a subject comprising administering an antimicrobial agent to the subject in combination with an immunomodulator composition comprising a nucleic acid sequence having at least 80% homology with SEQ ID NO: 1 and a lipid delivery vehicle, wherein the combination increases weight in the subject. In some aspects, the antimicrobial is an antibiotic such as those listed above. In some aspects, the antimicrobial is enrofloxacin.

Other aspects provide methods for increasing weight gain in a subject comprising administering an antimicrobial agent to the subject in combination with an immunomodulator composition comprising a nucleic acid sequence having at least 80% homology with SEQ ID NO:4 and a lipid delivery vehicle, wherein the combination increases weight in the subject. In some aspects, the antimicrobial is an antibiotic such as those listed above. In some aspects, the antimicrobial is enrofloxacin.

### II. Methods

An object of the present invention is to provide immunomodulator compositions, immunostimulatory plasmids (or DNA sequence), and methods that activate cytosolic DNA surveillance molecules to provide protective immunity to uninfected subjects, protective immunity to infected subjects, enhanced immunity to uninfected subjects, enhanced immunity to infected subjects, therapeutic immunity to infected subjects, or combinations thereof. As such, the compositions of the invention may be used to prophylactically immunize a subject or be used to treat a subject. The methods described herein include administrating an immunostimulatory plasmid, or DNA sequence, described herein to a subject, and activating cytosolic DNA surveillance molecules in the subject.

### A. Methods of Activating Cytosolic DNA Surveillance Molecules

The present invention is related to methods of activating cytosolic DNA surveillance molecules in a recipient subject. The methods comprise administering to a subject an effective amount of an immunomodulator composition described herein to activate cytosolic DNA surveillance molecules. In some aspects, the immunomodulator composition activates cytosolic DNA surveillance molecules. In some aspects, the immunomodulator composition enhances the operation of at least one biological agent such as a vaccine, when administered prior to such a vaccine, co-administered with a vaccine, administered post vaccination, or mixed with the vaccine. In some aspects, the methods provide new treatment strategies for protecting recipient subjects from infectious diseases and treating populations having infectious disease. In some aspects, the methods provide a more rapid, a longer and better protection against a disease when the immunomodulator is used in combination with a vaccine, compared to use of the vaccine without the immunomodulator composition.

Cytosolic DNA surveillance molecules can be activated in a recipient subject by administering an effective amount of an immunomodulator composition, which includes any of the liposome delivery vehicles described herein, any of the immunostimulatory plasmids (for DNA sequences) described herein, and optionally any of the biological agents described herein. It is contemplated that the biological agent may be mixed with or co-administered with the immunomodulator or independently thereof. Independent administration may be prior to or after administration of the immunomodulator. It is also contemplated that more than one administration of the immunomodulator or biological agent may be used. Furthermore, more than one biological agent may be co-administered with the immunomodulator, administered prior to the immunomodulator, administered after administration of the immunomodulator, or concurrently with the immunomodulator.

Any cytosolic DNA surveillance molecule known in the art or yet to be discovered may be modulated or activated using the immunomodulator compositions described herein. A skilled artisan will appreciate that such cytosolic DNA surveillance molecules are too numerous to list herein. As such, the immunomodulator compositions described herein may be used to activate or modulate any cytosolic DNA surveillance molecules capable of recognizing at least one immunomodulator component of the compositions described herein. By way of example, without limitation, such cytosolic DNA surveillance molecules include AIM2, AP1, ASC, Atg9a, B-catenin, caspase-1, cyclic GMP-AMP synthase (cGAS), DAI, DDX41, DEC205 DHX9, DHX36, DNA-PK, ERIS, IFI16, IKK complex, IKKε, IPSI, IRF1, IRF3, IRF7, ISRE1/7, ISRE7, JNK, Ku70, LGP2, LRRFIP1, MAPK, MDA-5, MITA, MKK3/6, MPYS, Mre11, Mx1, MyD88, NAP1, NFAT, NF-KB, NLRC5, OAS-3/OAS-L, pro-caspase-1, p38, RIG-I, RNA Pol III, SOCS1, SOCS3, STING, TANK, TBK1, TLR1, TLR2.1, TLR3, TLR7, TLR9, TLR21, TMEM173, TRAF3, TRAF6, TRAM, TRIF, TREX1, TRIM32, TRIM56, other interferon response factors known in the art, and combinations thereof.

An effective amount of any of the immunomodulator compositions described herein may be administered to a subject. The effective amount is sufficient to activate at least one (1) cytosolic DNA surveillance molecule in the recipient subject. Such effective amount is any amount that causes activation of at least one (1) cytosolic DNA surveillance molecule in a recipient subject. Methods of measuring such activation are known in the art. Also, a skilled artisan will recognize that the effective amount will depend upon age, weight, species of the subject and stage of infection, as well as other factors known in the art. Suitable effective amounts may range from about 0.1 μg to 1,000 μg per subject. In some aspects, the effective amount may range from about 0.1 μg to about 10 μg, from about 0.1 μg to about 5 μg, from about 0.5 μg to about 5 μg, from about 0.25 μg to about 5 μg, from about 0.05 μg to about 10 μg, from about 5 μg to about 15 μg, from about 10 μg to about 15 μg, from about 10 μg to about 20 μg, from about 20 μg to about 30 μg, from about 30 μg to about 40 μg, from about 40 μg to about 50 μg, from about 50 μg to about 70 μg, from about 70 μg to about 90 μg, from about 50 μg to about 100 μg, from about 100 μg to about 150 μg, from about 150 μg to about 200 μg, from about 200 μg to about 250 μg, from about 250 μg to about 300 μg, from about 300 μg to about 350 μg, from about 350 μg to about 400 μg, from about 400 μg to about 450 μg, from about 450 μg, to about 500 μg, from about 500 μg to about 550 μg, from about 550 μg to about 600 μg, from about 600 μg to about 650 μg, from about 650 μg to about 700 μg, from about 700 μg to about 750 μg, from about 750 μg to about 800 μg, from about 800 μg to about 850 μg, from about 850 μg to about 900 μg, from about 900 μg to about 950 μg, from about 950 μg to about 1000 μg. Preferably, in some aspects, the effective amount ranges from about 0.5 μg to about 10 μg. Yet, preferably in other aspects the effective amount ranges from about 50 μg to about 100 μg. And, preferably in other aspects, the effective amount ranges from about 40 μg to about 70 μg.

### B. Methods of Modulating an Immune Response

The immunomodulator compositions disclosed herein are particularly useful for modulating an immune response mounted by a recipient subject. Such methods of modulating an immune response in a subject include administering to the subject an effective amount of an immunomodulator composition described herein, activating immune surveillance receptors that activate signaling pathways involved in modulating an immune response. In some aspects, such methods may be used to stimulate an innate immune response. In some aspects, such methods may be used to stimulate an acquired immune response. In some aspects, such methods may be used to suppress an inflammatory immune response. In some aspects, such methods may be used to suppress inflammation during an immune response. In some aspects, such methods may be used to stimulate an innate immune response and suppress inflammation during the innate immune response. In some aspects, such methods may be used to stimulate an acquired immune response and suppress inflammation during the acquired immune response. In some aspects, such methods may be used to stimulate an innate immune response and an acquired immune response, while also suppressing inflammation.

### C. Conditions for Use

The methods of the invention activate at least one (1) cytosolic DNA surveillance molecule in a subject such that the subject is protected from a disease that is amenable to elicitation of an immune response. As used herein, the phrase “protected from a disease” refers to reducing the symptoms of the disease; reducing the occurrence of the disease; reducing the clinical or pathologic severity of the disease; or reducing shedding of a pathogen causing a disease. Protecting a subject can refer to the ability of a therapeutic composition of the present invention, when administered to a subject, to prevent a disease from occurring, cure, and/or alleviate or reduce disease symptoms, clinical signs, pathology, or causes. As such, protecting a subject from a disease encompasses both preventing disease occurrence (prophylactic treatment) and treating a subject that has a disease (therapeutic treatment). The term “disease” refers to any deviation from the normal health of a subject and includes a state when disease symptoms are present, as well as conditions in which a deviation (e.g., infection, gene mutation, genetic defect, etc.) has occurred, but symptoms are not yet manifested.

Methods of the invention may be used for the prevention of disease, stimulation of effector cell immunity against disease, elimination of disease, alleviation of disease, and prevention of a secondary disease resulting from the occurrence of a primary disease.

In some aspects, methods described herein may be used to improve the acquired immune response of the subject when co-administered with a vaccine versus administration of the vaccine by itself. Generally a vaccine once administered does not immediately protect the subject as it takes time to stimulate acquired immunity. The term “improve” refers, in the present invention, to elicitation of an innate immune response in the subject until the vaccine starts to protect the subject and/or to prolong the period of protection, via acquired immunity, given by the vaccine.

In some aspects, methods of the invention include administering the composition to protect against infection of a wide variety of pathogens. The composition administered may or may not include a specific antigen to elicit a specific response. It is contemplated that the methods of the invention will protect the recipient subject from disease resulting from infectious microbial agents including, without limitation, viruses, bacteria, fungi, and parasites. A skilled artisan will recognize and appreciate that a immunomodulator composition, as described herein, is effective against numerous infectious agents, which are too numerous to list. The infectious agents provided herein are provided for exemplary purposes and are provided without limitation of the scope of use.

### D. Administration

A variety of administration routes are available. The particular mode selected will depend, of course, upon the particular biological agents selected, the age and general health status of the subject, the particular condition being treated and the dosage required for therapeutic efficacy. The methods of this invention may be practiced using any mode of administration that produces effective levels of activation of cytosolic DNA surveillance molecules without causing clinically unacceptable adverse effects. The compositions may conveniently be presented in unit dosage form and may be prepared by any of the methods well known in the art.

The immunomodulator composition may be administered intravenously, intramuscularly, intramammary, intradermally, intraperitoneally, subcutaneously, by spray, in ovo by feather follicle method, orally, intraocularly, intratracheally, intranasally, mucosally, intrarectally, transdermally, by immersion (administration to aquatic species), or by other methods known in the art. In one aspect, the immunomodulator is administered subcutaneously. In another aspect, the immunomodulator may be administered intramuscularly. In another aspect, the immunomodulator is administered as a spray. In another aspect, the immunomodulator may be administered orally. In another aspect, the immunomodulator may be administered subcutaneously.

In one respect, the immunomodulator may be administered by itself to the subject prior to challenge (or infection). In another aspect, the immunomodulator may be administered by itself to the subject post challenge (or infection). In another aspect, the immunomodulator may be administered by itself to the subject at the same time as challenge (or infection).

In some aspects, the immunomodulator composition may be co-administered at the same time as the vaccination prior to challenge. In some aspects, the immunomodulator composition may be co-administered at the same time as the vaccination at the same time as challenge (or infection). In some aspects, the co-administration may include administering the vaccine and immunomodulator in the same general location on the subject at two different sites next to each other (i.e., injections next to each other at the neck of the subject), on opposing sides of the subject at the same general location (i.e., one on each side of the neck), or on different locations of the same subject. In some aspects, the immunomodulator composition can be administered prior to vaccination and challenge. In some aspects, the immunomodulator composition may be administered after vaccination but prior to challenge. The immunomodulator composition can be administered after challenge to a subject that has been vaccinated prior to challenge (or infection).

A skilled artisan will recognize that administration routes may vary depending upon the subject and the health or state of the subject. The administration routes provided for avian and bovine species are for exemplary purposes and are provided without limitation.

Vaccination of avian species may be performed at any age. Vaccinations may be administered to 18 day old embryos (in ovo) and above for a live microorganism and 3 weeks and older for an inactivated microorganism or other type of vaccine. For in ovo vaccination, vaccination may be administered in the last quarter of development. The vaccine may be administered subcutaneously, by the feather follicle method, by spray, orally, intraocularly, intratracheally, intranasally, in ovo, or by other methods know in the art. Oral vaccines may be administered in drinking water. Further, it is contemplated that the methods of the invention may be used based on routine vaccination schedules.

The immunomodulator composition may also be administered to an avian species subcutaneously, by the feather follicle method, by spray, intraocularly, intratracheally, intranasally, in ovo, or by other methods known in the art. For example, the immunomodulator composition can be administered in ovo. Alternatively, the immunomodulator composition can be administered as a spray.

The immunomodulator composition can be administered in ovo to an avian embryo in the last quarter of its development. For example, the immunomodulator composition can be administered in ovo to a 18-day-old or 19-day-old embryo. The administration to the egg may be prior to challenge (or infection) or post challenge.

The immunomodulator can be administered to an animal of the avian or bovine species from about 1 to about 14 days prior to challenge or from about 1 to about 14 days post challenge. For example, the immunomodulator can be administered from about 1 to about 7 days prior to challenge or from about 1 to about 7 days post challenge. The immunomodulator is suitably administered 1, 2, 3, 4, 5, 6, 7 days prior to challenge or 1, 2, 3, 4, 5, 6, 7 days post challenge.

Vaccination of bovine species may be performed at any age. The vaccine may be administered intravenously, intramuscularly, intradermally, intraperitoneally, subcutaneously, by spray, orally, intraocularly, intratracheally, intranasally, mucosally, intrarectally, transdermally, or by other methods known in the art. Further, it is contemplated that the methods described herein may be used based on routine vaccination schedules.

Other delivery systems may include time-release, delayed release, or sustained release delivery systems. Such systems can avoid repeated administrations of the compositions therefore increasing convenience. Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109.

Delivery systems also include non-polymer systems that are lipids including sterols such as cholesterol, cholesterol esters, and fatty acids or neutral fats such as mono-, di-, and tri-glycerides; hydrogel release systems; silastic systems; peptide based systems; wax coatings; compressed tablets using conventional binders and excipients; partially fused implants; and the like. Specific examples include, but are not limited to, erosional systems in which an agent of the invention is contained in a form within a matrix such as those described in U.S. Pat. Nos. 4,452,775, 4,675,189, and 5,736,152, and diffusional systems in which an active component permeates at a controlled rate from a polymer such as described in U.S. Pat. Nos. 3,854,480, 5,133,974, and 5,407,686. In addition, pump-based hardware delivery systems can be used, some of which are adapted for implantation.

As various changes could be made in the above composition, products, and methods without departing from the scope of the invention, it is intended that all matter contained in the above description and in the examples given below shall be interpreted as illustrative and not in a limiting sense.

### Definitions

The term “effective amount” refers to the amount necessary or sufficient to realize a desired biologic effect. For example, an effective amount of immunomodulator for treating or preventing an infectious disease is that amount necessary to cause the development of an immune response upon exposure to the microbe, thus causing a reduction in the amount of microbe within the subject and preferably the eradication of the microbe. The effective amount for any particular application can vary depending on such factors as the disease or condition being treated, the size of the subject, or the severity of the disease or condition. One of ordinary skill in the art can empirically determine the effective amount of immunomodulator without necessitating undue experimentation.

The term “cytokine” refers to an immune enhancing protein family. The cytokine family includes hematopoietic growth factor, interleukins, interferons, immunoglobulin superfamily molecules, tumor necrosis factor (TNF) family molecules and chemokines (i.e. proteins that regulate the migration and activation of cells, particularly phagocytic cells). Exemplary cytokines include, without limitation, interleukin-2 (IL-2), interleukin-12 (IL-12), interleukin-15 (IL-15), interleukin-18 (IL-18), interferon-α (IFNα), and interferon-γ (IFNγ).

The term “elicit” can be used interchangeably with the terms activate, stimulate, generate or upregulate.

The term “eliciting an immune response” in a subject refers to specifically controlling or influencing the activity of the immune response, and can include activating an immune response, upregulating an immune response, enhancing an immune response and/or altering an immune response (such as by eliciting a type of immune response which in turn changes the prevalent type of immune response in a subject from one which is harmful or ineffective to one which is beneficial or protective).

The term “operatively linked” refers to linking a nucleic acid molecule to a transcription control sequence in a manner such that the molecule is able to be expressed when transfected (i.e., transformed, transduced or transfected) into a host cell. Transcriptional control sequences are sequences which control the initiation, elongation, and termination of transcription. Particularly important transcription control sequences are those which control transcription initiation, such as promoter, enhancer, operator and repressor sequences. A variety of such transcription control sequences are known to those skilled in the art. Preferred transcription control sequences include those which function in avian, fish, mammalian, bacteria, viral, plant, and insect cells. While any transcriptional control sequences may be used with the invention, the sequences may include naturally occurring transcription control sequences naturally associated with a sequence encoding an immunogen or immune stimulating protein.

The terms “nucleic acid molecule” and “nucleic acid sequence” can be used interchangeably and include DNA, RNA, or derivatives of either DNA or RNA. The terms also include oligonucleotides and larger sequences such as plasmids, such as the immunostimulatory plasmids described herein, and including both nucleic acid molecules that encode a protein or a fragment thereof, and nucleic acid molecules that comprise regulatory regions, introns, or other non-coding DNA or RNA. Typically, an oligonucleotide has a nucleic acid sequence from about 1 to about 500 nucleotides, and more typically, is at least about 5 nucleotides in length. The nucleic acid molecule can be derived from any source, including mammalian, fish, bacterial, insect, viral, plant, synthetic sources or combinations thereof. A nucleic acid molecule can be produced by methods commonly known in the art such as recombinant DNA technology (e.g., polymerase chain reaction (PCR), amplification, cloning) or chemical synthesis. Nucleic acid molecules include natural nucleic acid molecules and homologues thereof, including, but not limited to, natural allelic variants and modified nucleic acid molecules in which nucleotides have been inserted, deleted, substituted, or inverted in such a manner that such modifications do not substantially interfere with the nucleic acid molecule's ability to elicit an immune response useful in the methods of the present invention. A nucleic acid homologue may be produced using a number of methods known to those skilled in the art (see, for example, Sambrook et al., Molecular Cloning: A Laboratory Manual, Cold Spring Harbor Labs Press, 1989), which is incorporated herein by reference.

The terms “selectable marker” and “selectable marker gene” refer to a gene that encodes a product that protects the organism in which the gene is expressed from a selective agent (e.g., an antibiotic) or a condition that would normally kill the organism or inhibit its growth. Selectable marker genes are most commonly antibiotic resistance genes (e.g., kanamycin resistance genes, ampicillin resistance genes, chloramphenicol resistance genes, tetracycline resistance genes, etc.). Thus, for example, when E. coli cells are subjected to a transformation procedure to introduce a plasmid encoding a kanamycin resistance gene and then grown on or in media containing kanamycin, only the E. coli cells that have successfully taken up the plasmid and expressed the kanamycin resistance gene will survive. The terms “selectable marker” and “selectable marker gene” also include genes that code for enzymes involved in the synthesis of a compound that is essential for the growth of an organism. When introduced into an auxotrophic organism that is unable to synthesize the essential compound, such genes allow the organism to grow in a medium that has been supplemented with the essential compound. For example, bacterial cells that are auxotrophic for the amino acid lysine due to a mutation in or the absence of an enzyme involved in lysine biosynthesis normally are unable to grown on media that has not been supplemented with lysine. When such bacteria are subjected to a transformation procedure to introduce a plasmid encoding the enzyme involved in lysine biosynthesis, the bacteria that have successfully taken up the plasmid and expressed the enzyme will survive when grown on media that has not been supplemented with lysine. The terms “selectable marker” and “selectable marker gene” further include genes that allow for poison/antidote selection. For example, the ccdB gene encodes a protein that binds to DNA gyrase, an essential enzyme for cell division. Upon binding to DNA gyrase, the ccdB gene product impairs gene replication and induces cell death. Thus, bacterial expressing the ccdB gene product cannot survive. The ccdA gene encodes a protein (the “antidote”) that acts as a natural inhibitor of the ccdB gene product. Thus, when bacteria having the ccdB gene in their bacterial genome are subjected to a transformation procedure to introduce a plasmid encoding the ccdA gene product, only the cells that successfully take up the plasmid and express the ccdA gene will survive.

The terms “screenable marker” and “screenable marker gene” refer to a gene that encodes a product that allows an observer to distinguish between cells expressing the screenable marker gene and cells that are not expressing the screenable marker gene. Screenable marker gene systems are well known in the art and include, for example, lacZ genes and genes encoding fluorescent proteins such as green fluorescent protein (GFP), yellow fluorescent protein (YFP), red fluorescent protein (RFP), blue fluorescent protein (BFP), or cyan fluorescent protein (CFP).

As used herein, the term “subject” refers to a living organism having a central nervous system. In particular, subjects include, but are not limited to, human subjects or patients and companion animals. Exemplary companion animals may include domesticated mammals (e.g., dogs, cats, horses), mammals with significant commercial value (e.g., avian species, bovine species, dairy cows, beef cattle, sporting animals), mammals with significant scientific values (e.g., captive or free specimens of endangered species), or mammals which otherwise have value. Suitable subjects also include: mice, rats, dogs, cats, ungulates such as cattle, swine, sheep, horses, and goats, lagomorphs such as rabbits and hares, other rodents, and primates such as monkeys, chimps, and apes. Subjects may be any member of the avian species, whether domestic or wild, and may be commercially reared for breeding, meat or egg production. Exemplary avian species include, without limitation, chickens, turkeys, geese, ducks, pheasants, quail, pigeons, ostriches, caged birds, birds in zoological collections and aviaries and the like. Subjects may be any member of the bovine species, whether domestic or wild, and may be commercially reared for breeding, meat or mil production. Exemplary bovine species include, without limitation, antelopes, buffalos, yaks, cattle, bison, and the like. Species of cattle include, without limitation, cows, bulls, steers, heifer, ox, beef cattle, dairy cattle, and the like. Subjects may be any member of an aquaculture species, including without limitation, any species of fish, crustaceans, molluscs, living in freshwater or saltwater. In some aspects, subjects may be diagnosed with an infectious disease, may be at risk for an infectious disease, or may be experiencing an infectious disease. Subjects may be of any age including in utero, new born, adolescence, adult, middle age, or elderly.

### EXAMPLES

The following non-limiting examples are provided to further illustrate the present invention.

### Example 1: Activating Cytosolic DNA Surveillance Molecules with Immunomodulator Compositions Using Monocyte Cell Line

Immunomodulator compositions described herein were used to activate interferon regulatory factor 3 (IRF-3), a transcription factor activated by DNA surveillance molecules. A human macrophage-like (monocyte) cell line (THP-1), derived from an acute monocytic leukemia patient, is used as a model system for monocyte function. THP1-Blue ISG cells (Invitrogen) were generated by stable integration of an interferon regulatory factor (IRF)-inducible secretory alkaline phosphatase (SEAP) reporter construct (IRF-THP1 cells). The THP-1 cells endogenously contain functional STING (Simulator of interferon genes) pathway molecules, as well as cytosolic DNA recognition receptors. STING receives signals from and is itself a cytosolic DNA surveillance molecule that acts through IRF-3. Activation of STING leads to SEAP production, which is then detectable in the culture supernatant. Thus, activation of the IRF-3 reporter SEAP construct correlates to activation of the STING pathway.

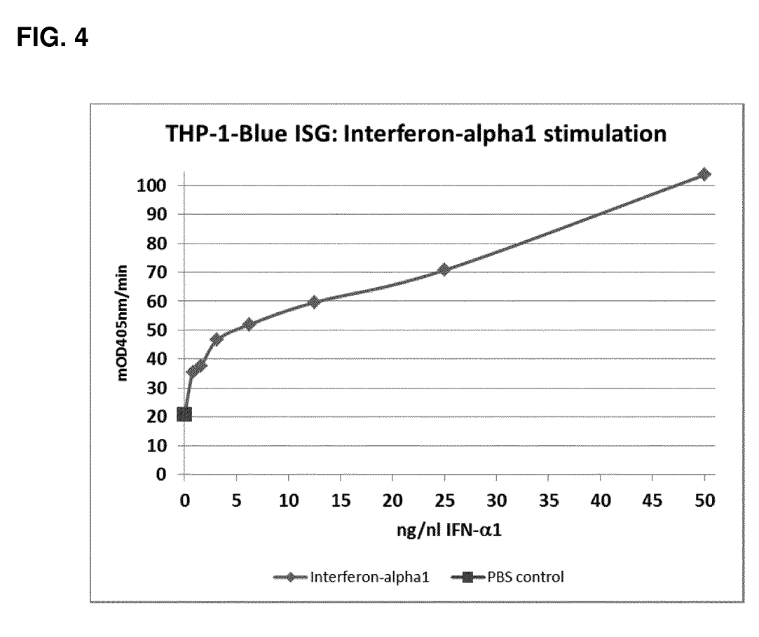

The stably transfected IRF-THP-1 cell line was tested for its functionality by IFN-α1, which is a global activator of different IRF pathways. IRF-THP-1 cells were contacted with human interferon α1 (IFN α1) as a positive control for IRF-3 activation. A specific SEAP signal was detected depending on IFN-α1 dosing and a clear dose-response relationship was observed. FIG. 4 graphically illustrates IFN α1 activation of IRF-3. The IRF-dependent signaling axis is functional in the IRF-THP-1 cells line.

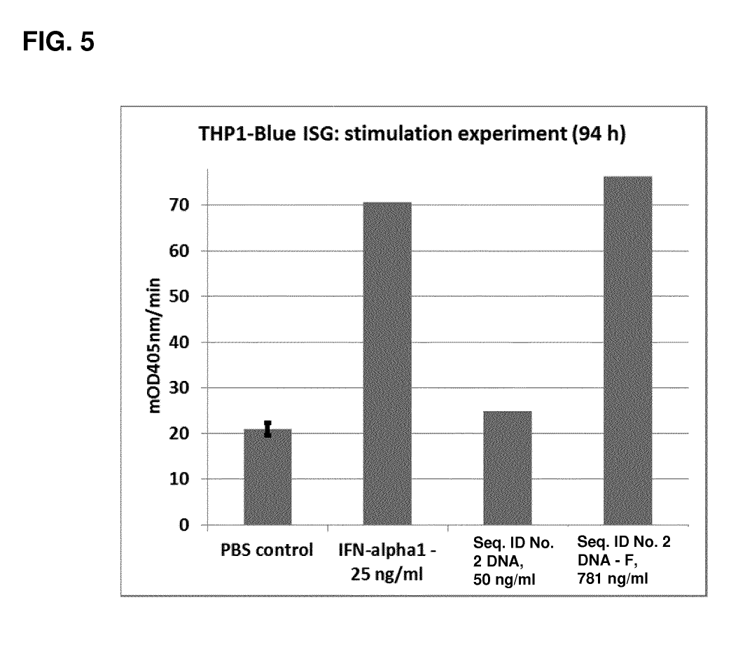

An initial experiment with SEQ ID NO. 2 as unformulated plasmid and as a liposome-formulated plasmid (FIG. 5, Seq No 2-F) suggested that the SEQ ID NO. 2 unformulated does not elicit a specific signal even at very high concentrations (50 μg/mL). In contrast, Seq No 2-F gives rise to a SEAP signal in the ng/mL range with a comparable magnitude to the IFN-α1 control (FIG. 5).

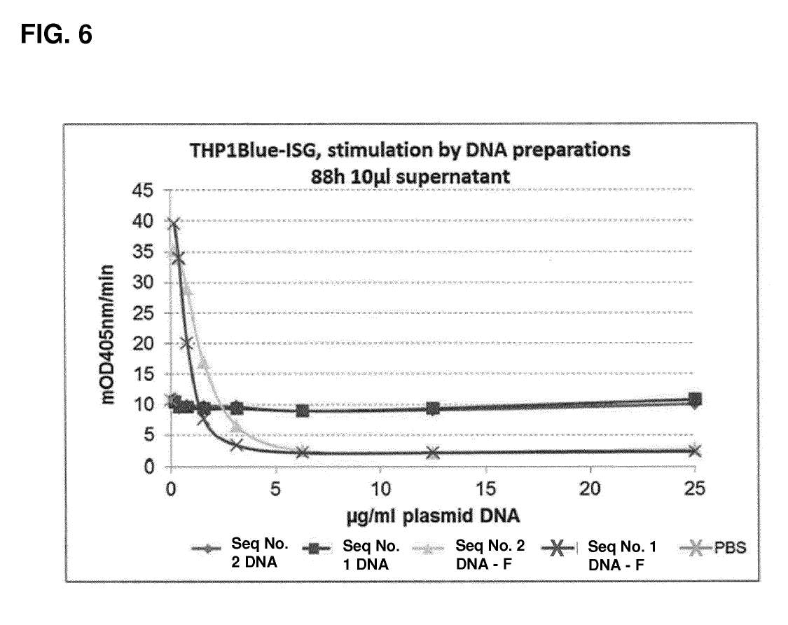

IRF-THP-1 cells were contacted with immunomodulator compositions described herein. The immunomodulator compositions included SEQ ID NO. 2 unformulated (Seq No 2), SEQ ID NO. 1 unformulated (Seq No 1), SEQ ID NO. 2-formulated with liposome (DOTIM/cholesterol) carrier (Seq No 2-F), SEQ ID NO. 1 formulated with liposome (DOTIM/cholesterol) carrier (Seq No 1-F), and PBS (negative control). The DNA alone, without the liposome component, did not activate IRF-3 (FIG. 6). The DNA/liposome compositions activated IRF-3 at the lowest concentration (FIG. 6).

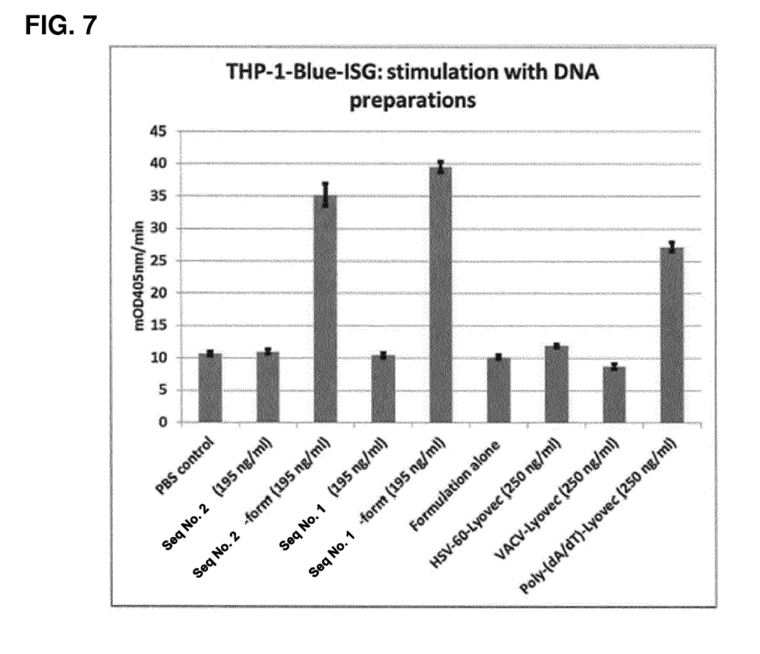

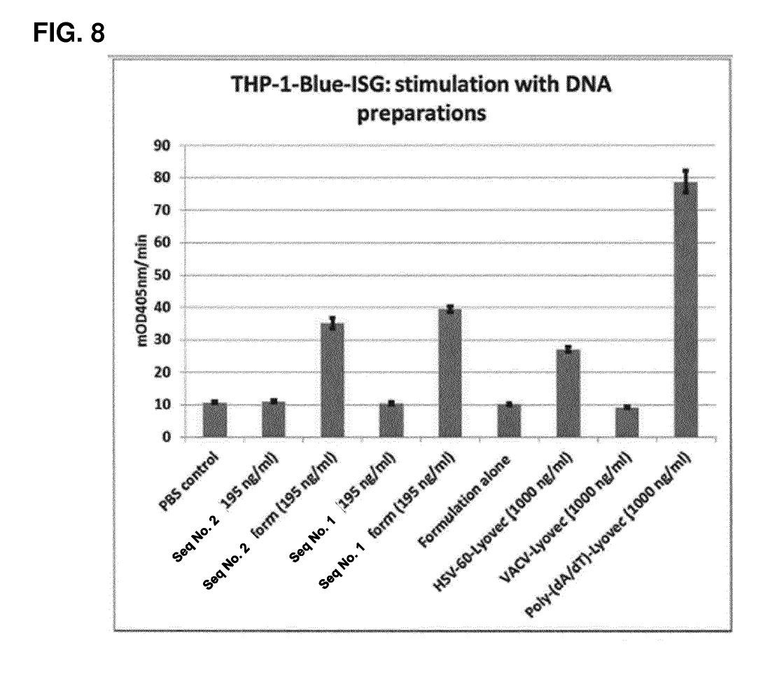

IRF-THP-1 cells were contacted with immunomodulator compositions described herein and controls. The immunomodulator compositions included SEQ ID NO. 2 unformulated (Seq No 2), SEQ ID NO. 1 unformulated (Seq No 1), SEQ ID NO. 2-formulated with liposome (DOTIM) carrier (Seq No 2-F), and SEQ ID NO. 1 formulated with liposome (DOTIM) carrier (Seq No 1-F). The controls included PBS (negative control), liposome component (formulation alone), HSV-60-Lyovec (known cytosolic DNA recognition activator), VACV-Lyovec (known cytosolic DNA recognition activator), and Poly-(dA/dT)-Lyovec (known cytosolic DNA recognition activator). The DNA alone, without the liposome component, did not activate IRF-3 (FIG. 7 and FIG. 8). The DNA/liposome compositions activated IRF-3 (FIG. 7 and FIG. 8). The liposome component on its own did not have an effect on the IRF3 reporter gene expression (FIG. 7 and FIG. 8).

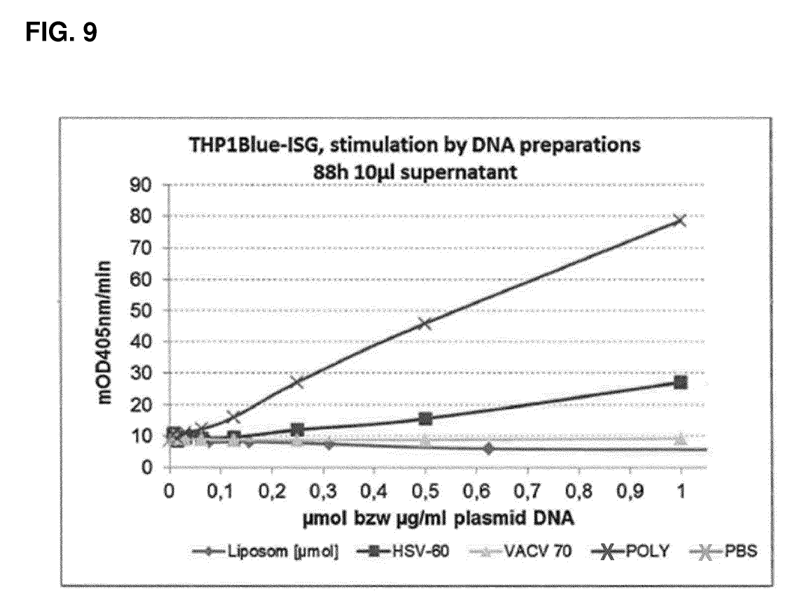

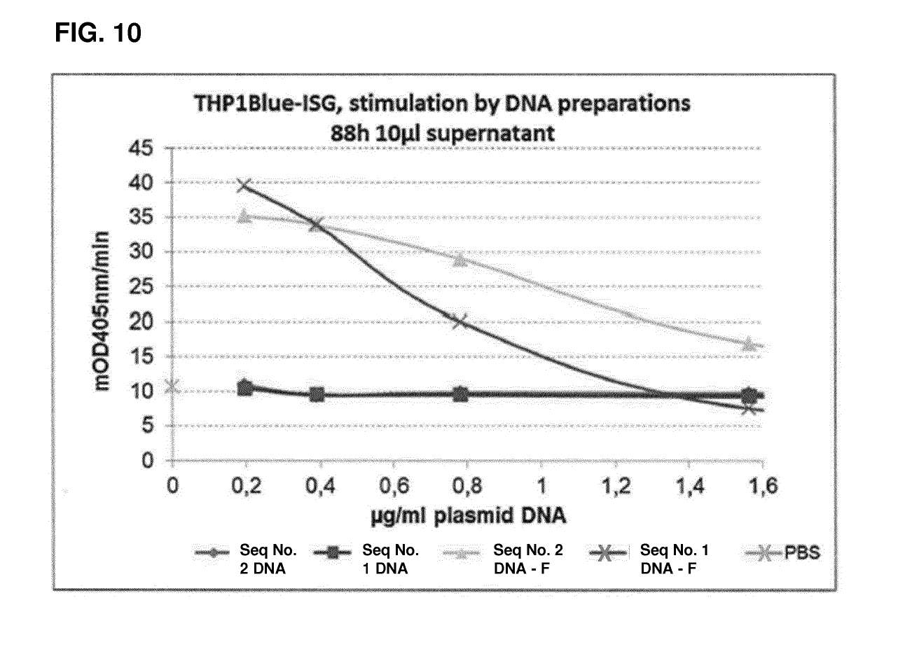

The immunomodulator compositions described herein activated cytosolic DNA surveillance molecules similar to, and in some cases better than, the activation by known cytosolic DNA recognition activators (FIG. 9 and FIG. 10).

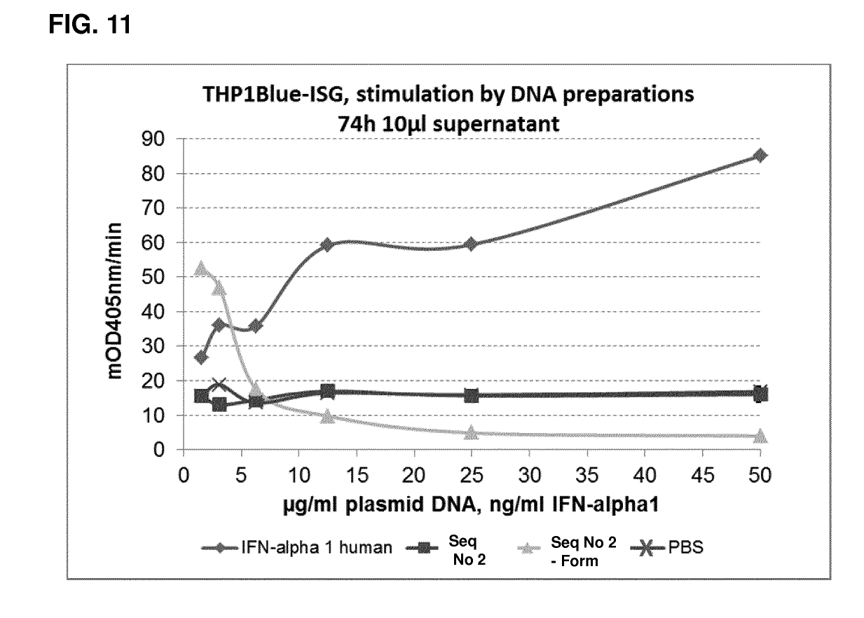

Dose-response curves of SEQ ID NO. 2 as unformulated plasmid and as liposome-formulated plasmid (Seq No 2-F) confirmed that the “naked” plasmid (Seq No 2) is identical to the PBS dilution control over the entire concentration range considered (FIG. 11; 1.5625-50 μg/ml), and, thus, is not stimulatory. By contrast, Seq No 2-F gave rise to a SEAP signal at the highest dilutions (1.5625 μg/ml and 3.125 μg/ml), while higher concentrations led to a loss of SEAP secretion capacity, even below the PBS control. Microscopic inspection suggests that cytotoxicity of Seq No 2-F at higher concentrations may be the reason for this effect. The positive control Interferon-α1 gave rise to a dose-dependent response.

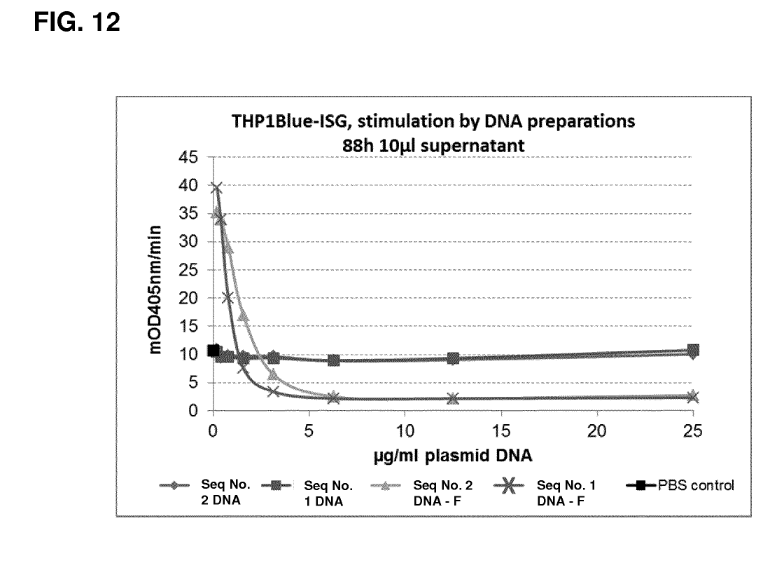

Dose-response curves of Seq No 2 and Seq No 1 as unformulated plasmid and as liposome-formulated plasmids (Seq No 2-F, Seq No 1-F) confirmed that both unformulated plasmid do not show a specific signal over the entire concentration range considered (0.39-25 μg/ml) (FIG. 12). By contrast, Seq No 2-F and Seq No 1-F gave rise to SEAP signals at the highest dilutions (0.39 μg/ml, 0.78 μg/ml and 1.5625 μg/ml), while higher concentrations led to a loss of SEAP secretion capacity, even below the PBS control. Microscopic inspection suggests that cytotoxicity of Seq No 2-F and Seq No 1-F at higher concentrations may be the reason for this effect.

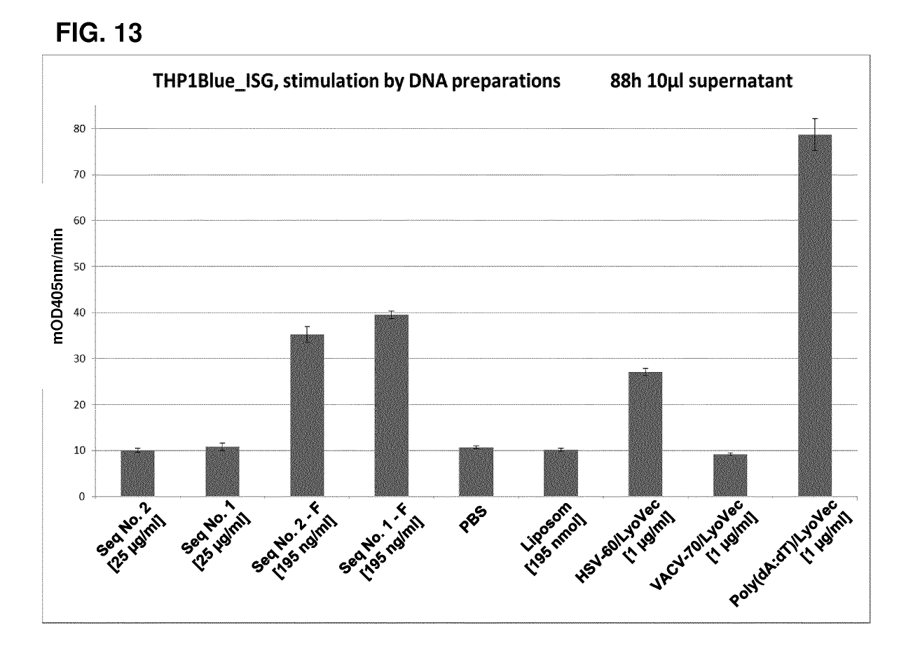

Seq No 2 and Seq No 1 as unformulated plasmids show no specific signal over the PBS background, even at 25 μg/ml (FIG. 13). By contrast, liposome-formulated plasmids (Seq No 2-F, Seq No 1-F) showed marked stimulation at 195 ng/ml, while a liposome control showed no signal. In comparison to the standard ligands HSV-60-LyoVec and VACV-70-LyoVec addressing the STING pathway, Seq No 2-F and Seq No 1-F showed a stronger stimulatory capacity. Poly(dA/dT)/LyoVec that addresses several different cytoplasmic recognition pathways showed the strongest signal in this test system, albeit at higher concentration.

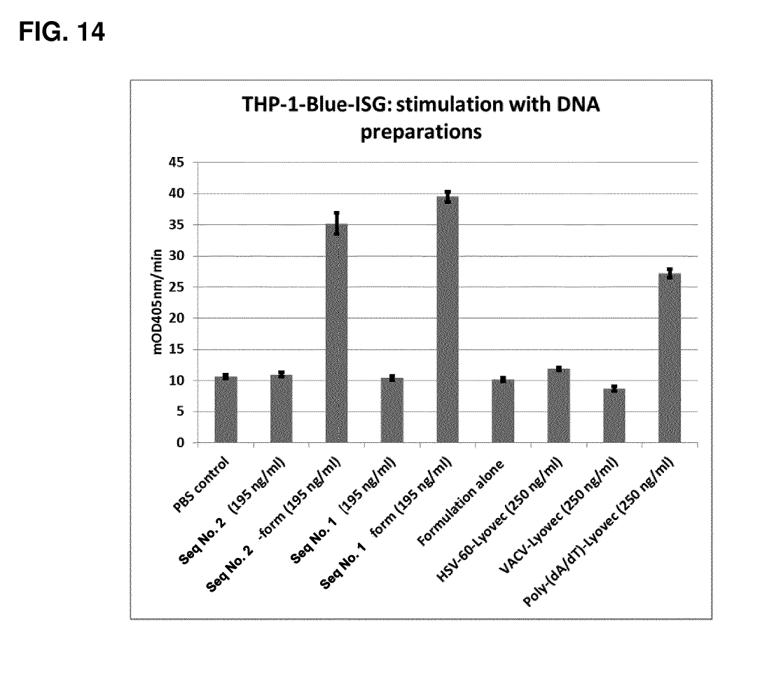

Seq No 2 and Seq No 1 as unformulated plasmids show no specific signal over the PBS background. By contrast, liposome-formulated plasmids (Seq No 2-F, Seq No 1-F) showed marked stimulation at identical concentration, while a liposome control showed no signal (FIG. 14). In comparison to the standard ligands HSV-60-LyoVec and VACV-70-LyoVec as well as Poly(dA/dT)/LyoVec addressing the STING pathway, Seq No 2-F and Seq No 1-F showed a stronger stimulatory capacity when applied in similar concentrations.

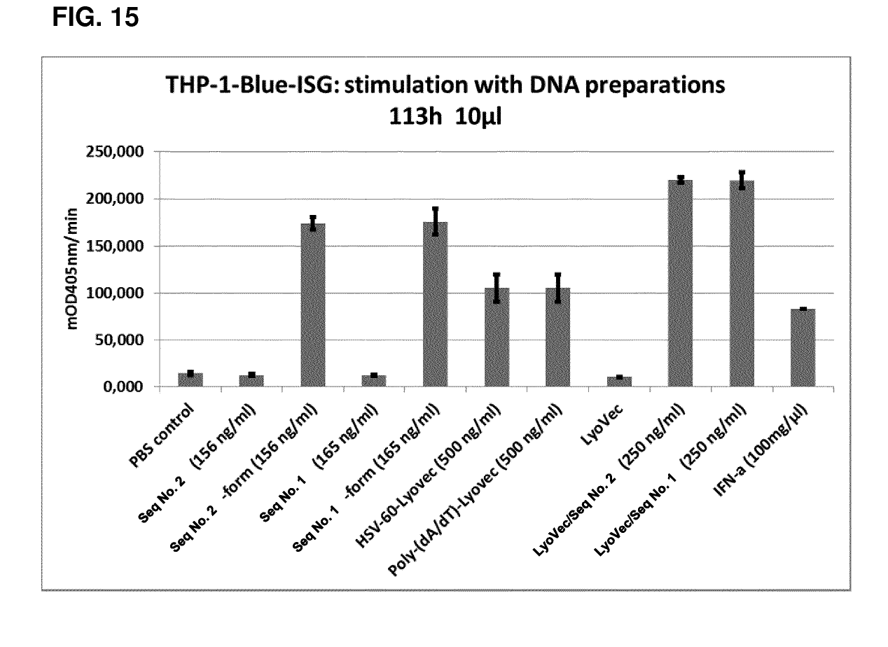

As shown in FIG. 14, Seq No 2 and Seq No 1 as unformulated plasmids show no specific signal over the PBS background. By contrast, liposome-formulated plasmids (Seq No 2-F, Seq No 1-F) exhibited marked stimulation at identical concentration, while a liposome control showed no signal. In comparison to the standard ligands HSV-60-LyoVec as well as Poly(dA/dT)/LyoVec addressing the STING pathway, Seq No 2-F and Seq No 1-F showed a stronger stimulatory capacity when applied in similar concentration (FIG. 15). The transfection agent LyoVec by itself does not stimulate IRF-THP-1 cells. When the unformulated plasmids Seq No 2 and Seq No 1 are applied together with LyoVec as the complexation component, stimulation of the IRF pathway is apparent.

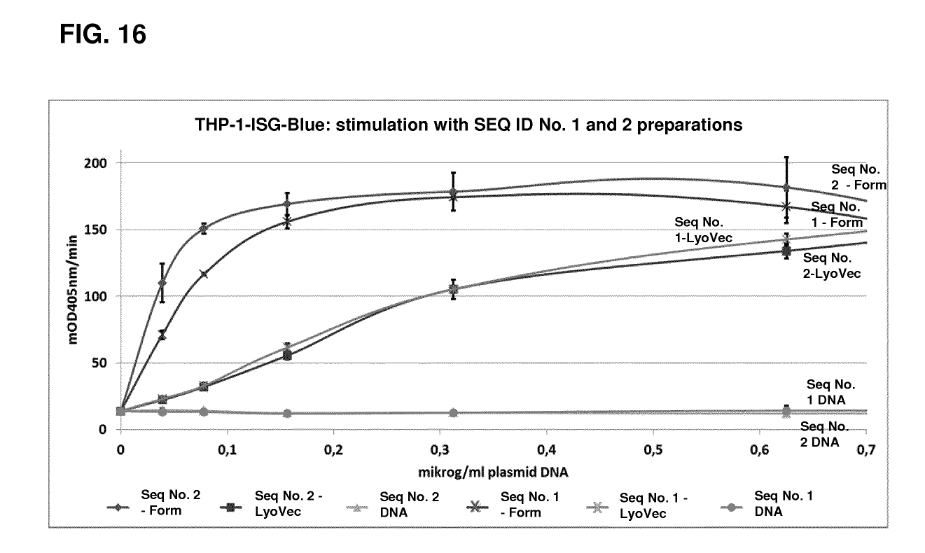

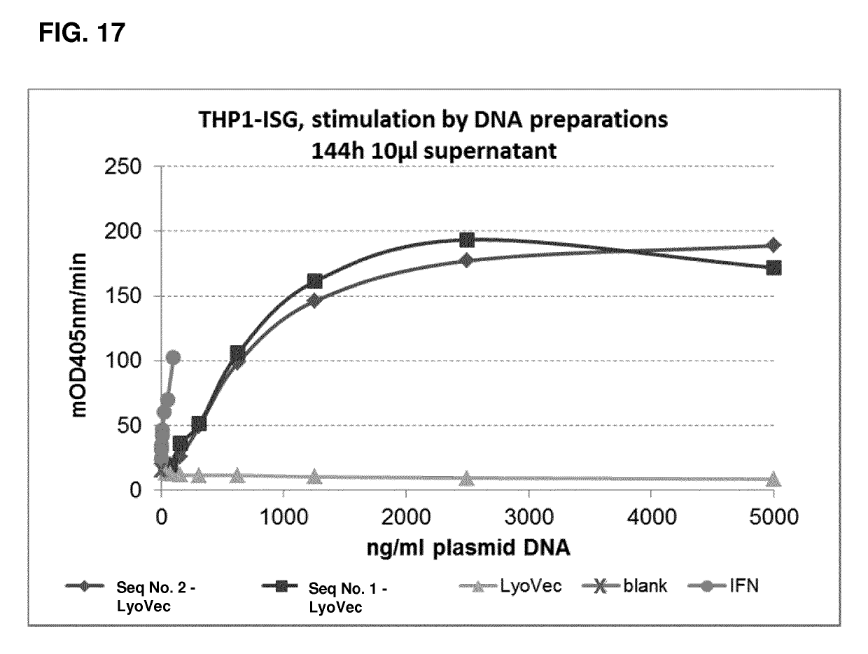

Dose response curves of Seq No 2 and Seq No 1 in the IRF-THP-1 reporter gene system as unformulated, liposome-formulated version (Seq No 2-F and Seq No 1-F) and as LyoVec-formulated versions were generated in the sub-μg/ml concentration range (FIG. 16). The unformulated plasmids were inactive, while the liposome-formulated versions showed a dose response superior to the LyoVec formulations, suggesting that the liposomes are superior formulations in the low concentration range.

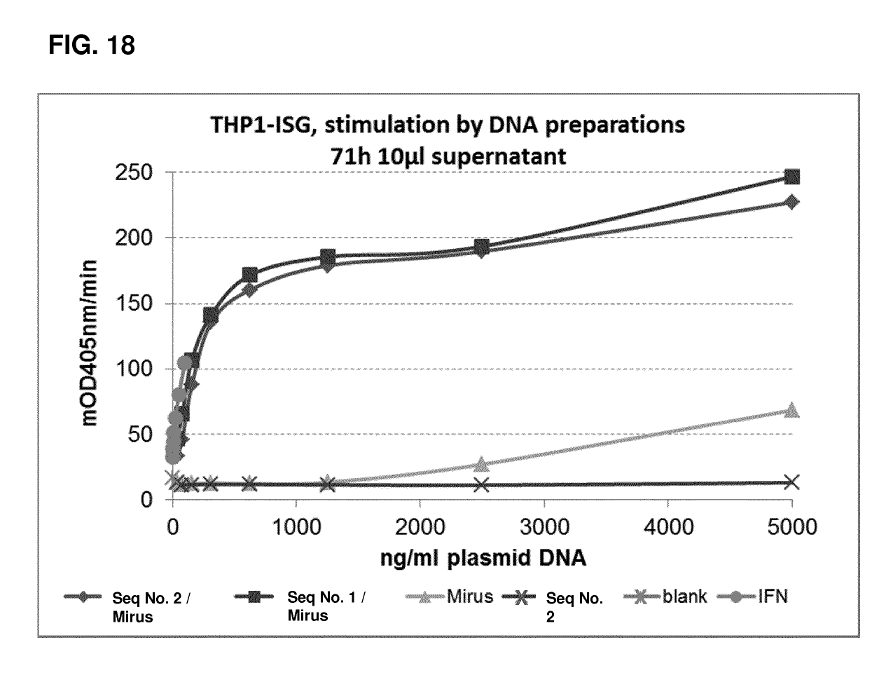

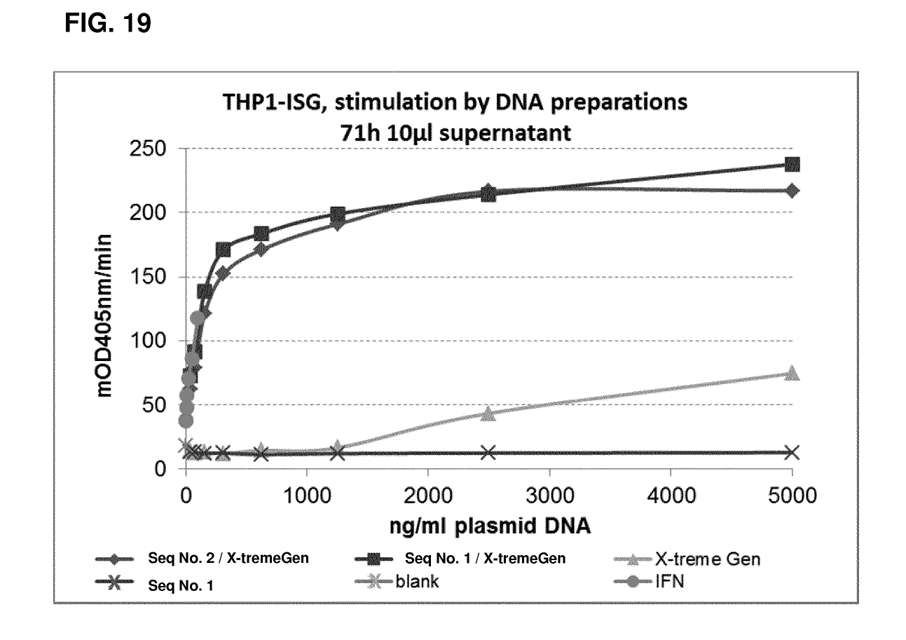

Dose response curves of Seq No 2 and Seq No 1 in the IRF-THP-1 reporter gene system as unformulated, transfection agent-formulated (LyoVec (FIG. 17), Mirus (FIG. 18), X-tremeGen (FIG. 19)) versions were generated in the sub-μg/ml concentration range. The unformulated plasmids were inactive, while the transfection agent-formulated versions showed IRF stimulation signals with clear dose response. This is a further indication for the stimulatory potential of the plasmids on the cytoplasmic DNA recognition mechanisms of THP-1 cells.

### Example 2: Activating Cytosolic DNA Surveillance Molecules with Immunomodulator Compositions Using Melanoma Cell Line

B16-Blue™ ISG cells were derived from the murine B16 F1 melanoma cell line. They express the secreted embryonic alkaline phosphatase (SEAP) reporter gene under the control of the I-ISG54 promoter which is comprised of the IFN-inducible ISG54 promoter enhanced by a multimeric ISRE. Stimulation of B16-Blue™ ISG cells with IFNs, cyclic dinucleotides, such as cGAMP, or type I IFN inducers, such as transfected poly(dA:dT), triggers the activation of the I-ISG54 promoter and the production of SEAP.

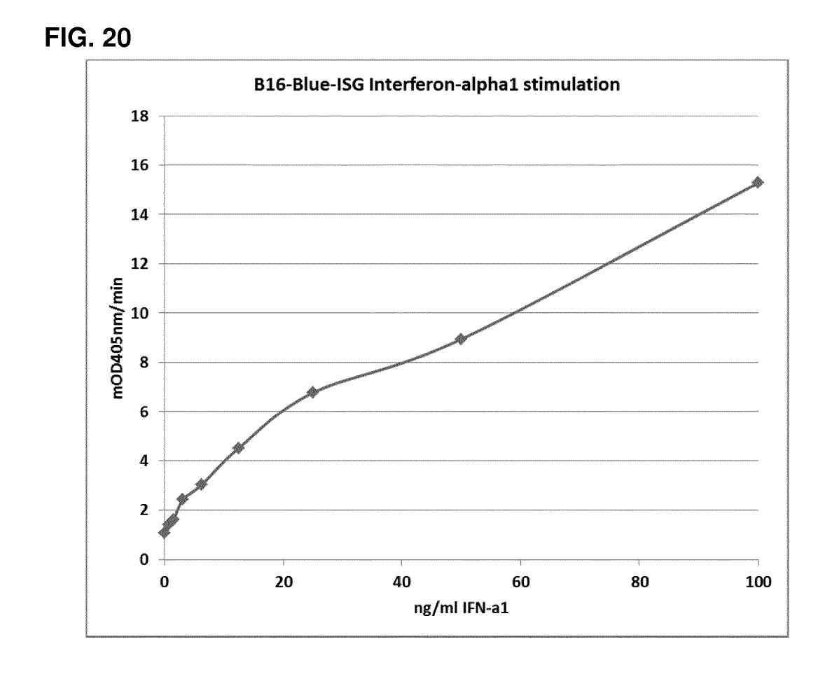

The B16-Blue™ ISG cell line was tested for its functionality by IFN-α1, which is a global activator of different IRF pathways. A specific SEAP signal was detected depending on IFN-α1 dosing, and a clear dose-response relationship was apparent (FIG. 20). This experiment suggested that the IRF-dependent signaling axis is functional in this cell line.

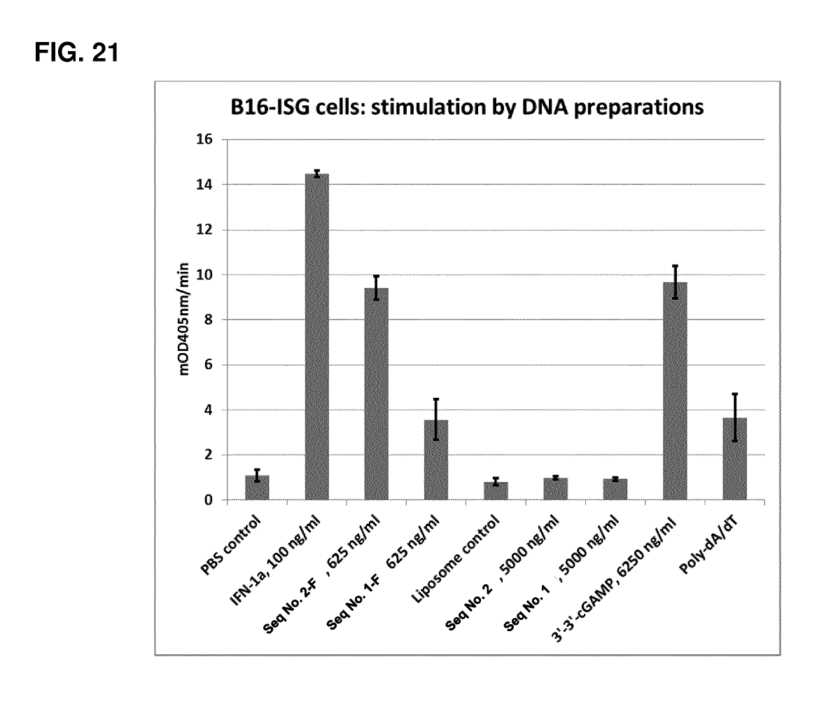

B16-Blue™ ISG cells were stimulated by the liposome formulated plasmids Seq No 2-F and Seq No 1-F at 625 ng/ml, while the unformulated plasmids showed no specific signal at 8-fold higher concentration (5 μg/ml) compared to the PBS control (FIG. 21). Likewise, liposomal formulation alone was not stimulatory. The controls 3′,3′-cGAMP and poly-dA/dT showed the expected specific signals.

### Example 3. Activating Cytosolic DNA Surveillance Molecules with Immunomodulator Compositions Using STING Knockout Cell Line

THP1-Blue™ ISG-KD-STING cells were generated from THP1-Blue™ ISG cells through knockdown of the STING gene expression. As a result, THP1-Blue™ ISG-KD-STING cells display a considerable reduction of STING expression.

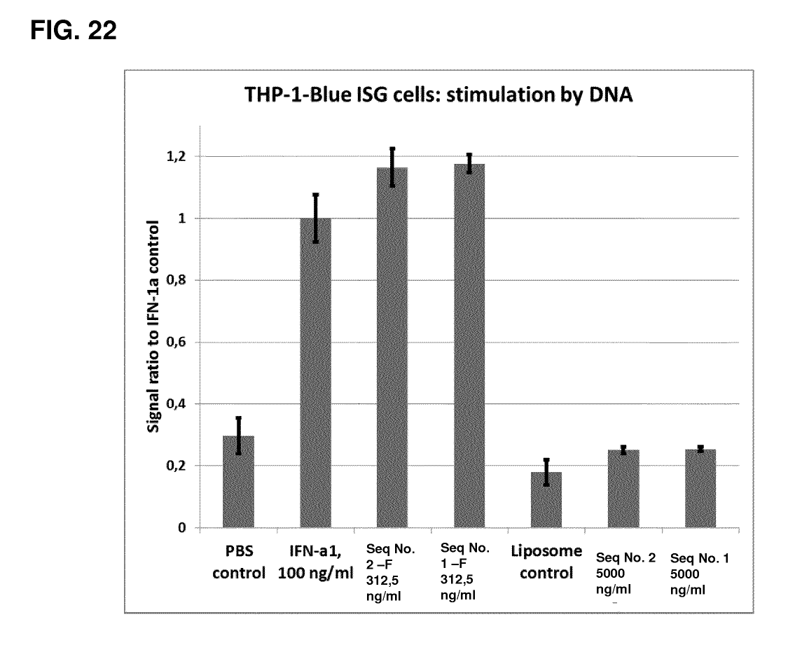

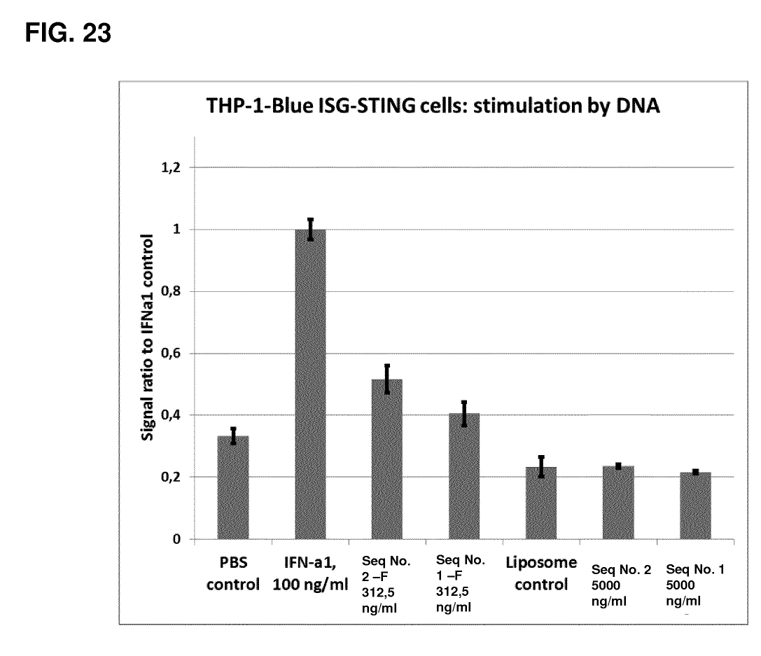

In a comparative experiment, interferon-α1 (IFN-α1) was used as a control, as its signaling to IRFs is not dependent on STING. Signals of other test compounds were normalized to the IFN-α1 signal set as 1. Seq No 2 and Seq No 1 as unformulated plasmids and liposomal formulation alone showed no specific signal over the PBS background at 5 μg/ml, neither in THP-1-Blue™ ISG (FIG. 22) or in THP-1-Blue™ ISG-STING cells (FIG. 23). By contrast, liposome-formulated plasmids (Seq No 2-F, Seq No 1-F) showed marked stimulation of THP-1-Blue™ ISG cells at 312.5 ng/ml, while in THP-1-Blue™ ISG-STING this signal was downregulated by 67% (Seq No 2-F) up to 91% (Seq No 1-F). These results indicate that the STING-mediated pathway is one cytosolic DNA surveillance pathway that is activated by immunomodulators described herein.

### Example 4: Activating Cytosolic DNA Recognition with Immunodulator Compostions Using a Human Monocytic Cell Line and a Murine Melanoma Cell Line

Cytosolic DNA recognition of the Seq No 2 immunomodulator was explored using the THP1-Blue™ and B16-Blue™ cell lines. Cell cultures were treated with Seq No 2-F, unformulated Seq No 2, or appropriate control compositions.

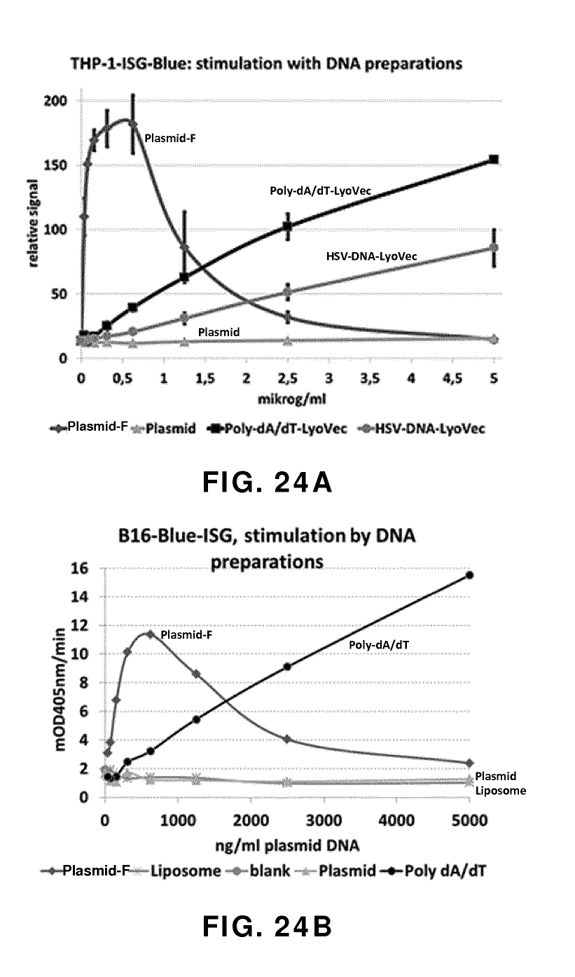

The THP1-Blue™ cell line was stimulated by the liposome formulated Seq No 2-F. SEAP signals for the Seq No 2-F treated cells were approximately four times greater than for cells treated with a positive control to generate SEAP signals. However, THP1-Blue™ cells treated with the unformulated plasmids showed no specific signal at any concentration tested (FIG. 24A).

Similarly, the B16-Blue™ cell line was also stimulated by Seq No 2-F treatment, but not with unformulated plasmid. Stimulation with Seq No 2-F generated greater signal at lower concentrations than stimulation with the positive control (FIG. 24B). These results show that Seq No 2-F is a potent, activating ligand for cytosolic DNA recognition.

### Example 5: Activating Cytosolic DNA Surveillance Molecules with Immunomodulator Compositions Using STING Knockout and Knockdown Cell Lines

B16-Blue™ ISG-KO-STING cells were generated from the B16-Blue™ ISG cell line, a murine B16-F1 melanoma-derived cell line, through stable knockout of the STING gene. They express the secreted embryonic alkaline phosphatase (SEAP) reporter gene under the control of the I-ISG54 promoter which is comprised of the IFN-inducible ISG54 promoter enhanced by a multimeric ISRE. These cells do not respond to cytosolic DNA, DMXAA, canonical and non-canonical CDNs while retaining the ability to respond to type I and type II IFNs. Stimulation of these cells with IFN triggers the activation of the I-ISG54 promoter and the production of SEAP.

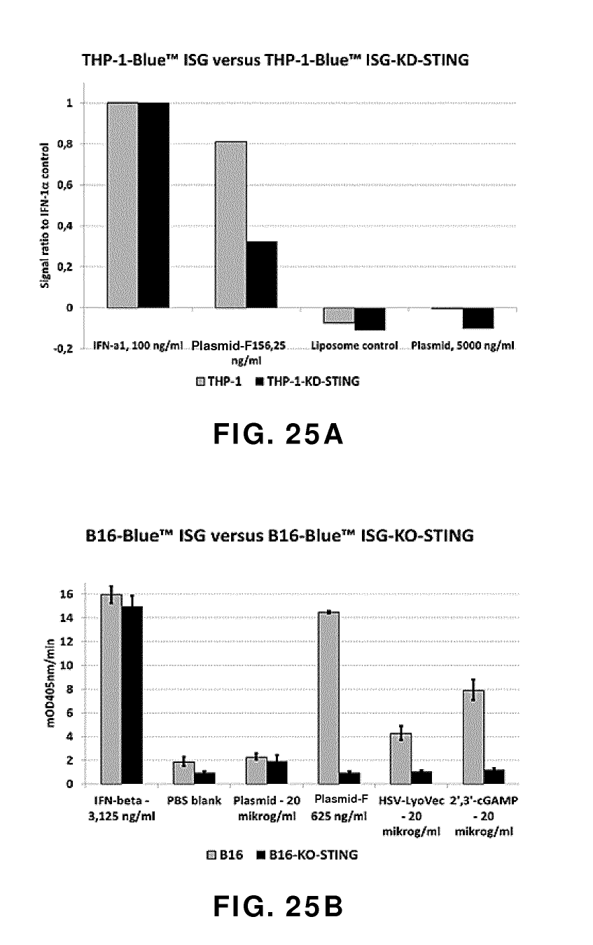

Treatment of THP-1-Blue™ ISG-KD-STING cells and THP-1-Blue™ cells with Seq No 2-F was compared. The SEAP signal generated in the knock-down cells was less than 50% of the signal generated in the THP-1-Blue™ cells (FIG. 25A). Treatment of B16-Blue™ ISG-KO-STING cells with Seq No 2-F was also compared to treatment of B16-Blue™ with Seq No 2-F. While treatment of the B16-Blue™ cells resulted in a SEAP signal similar to that of the positive controls (FIG. 25B), treatment of the knockout cells generated no signal beyond that of a PBS control (FIG. 25B).

These results suggest that Seq No 2-F activation of the IRF pathway is STING-dependent.

### Example 6: Comparing Activation of Cytosolic DNA Surveillance Molecules with Immunodulator Compositions Using a STING Knockout Cell Line and a STING Wildtype Cell Line

A STING knockout cell line and a STING wildtype cell line were used to determine if STING is essential for Seq No 2-F and Seq No 1-F recognition.

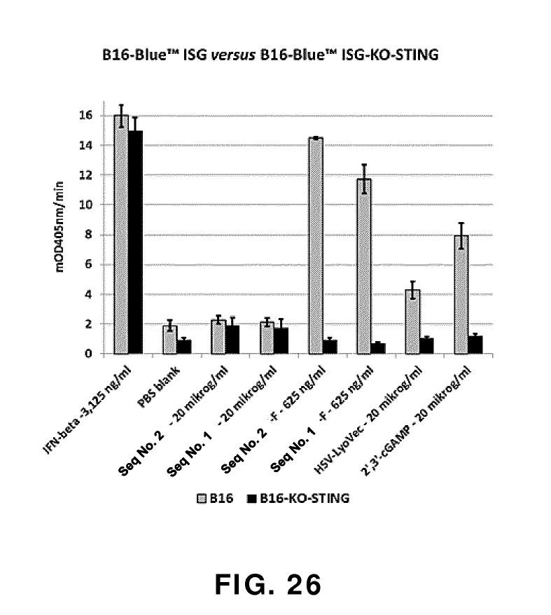

These cell lines were validated first by incubation with interferon-β (IFN-β), a cytokine whose signal transduction pathway should not be affected by deletion of the STING gene, which is the observation made in our experiments. Referring to FIG. 26, known STING pathways activators, Herpes simplex virus DNA complexed with LyoVec (HSV-LyoVec) and the cyclic dinucleotide 2′,3′-cGAMP were used to validate cell line properties. While both agents produced significant signals in the parental B16-Blue ISG cell line, these signals were completely abrogated in B16-Blue ISG-KO-STING cells.

Application of uncomplexed Seq No 2 or Seq No 1 plasmids gave no or only minor signals, relative to a PBS control, in both cell lines. Liposome-complexed SEQ ID NO. 2 (Seq No 2-F) and SEQ ID NO. 1 (Seq No 1-F) resulted in strong signals in the parental B16-Blue ISG cell line, while no signal was observed in B16-Blue ISG-KO-STING cells.

Taken together, the data suggest, that at least in murine B16 melanoma cells with an interferon-response element readout, the STING pathway is essential for recognition of Seq No 2-F and Seq No 1-F.

### Example 7: Ex Vivo Induction of Interferon Release in Pigs

The purpose of this study was to determine the effect of Seq No 2-F on the interferon production in peripheral blood mononuclear cells (PMBCs) isolated from pigs.

Vesicular stomatitis virus (VSV) cytolytic assays were employed to detect type 1 interferon expression in the isolated PMBCs. The VSV assays were performed on PMBCs isolated from three separate pigs. The PMBCs were treated with formulated Seq No 2-F both with and without costimulus (UV-inactivated herpes virus). Referring to Table 1, the analysis was carried out at differing concentrations of Seq No 2 over the course of 2, 4 and 6 days.

TABLE 1
Interferon release from porcine PMBCs

| | | | Concentration μg/ml | | | | | | | | |
|---|---|---|---|---|---|---|---|---|---|---|---|
| | | | IFN | | | | | | | | |
| Pig | | | 100 | 50 | 25 | 12.5 | 6.25 | 3.1 | 1.56 | 0.78 | 0.4 |
| Seq No 2-F | | | — | — | — | — | — | 3 | 1.5 | 0.8 | 0.4 |
| Test I | Day 2 | EU/ml | tox | tox | tox | 160 | 640 | 1280 | 2560 | 2560 | 2560 |
| Tier 2008 | | pg/ml | tox | tox | tox | 3.25 | 13 | 26 | 52 | 52 | 52 |
| fesh | Day 4 | EU/ml | tox | tox | tox | 40 | 320 | 640 | 1280 | 2560 | 2560 |
| | | pg/ml | tox | tox | tox | 0.8 | 6.5 | 13 | 26 | 52 | 52 |
| | Day 6 | EU/ml | tox | tox | tox | 20 | 160 | 640 | 640 | 1280 | 2560 |
| | | pg/ml | tox | tox | tox | 0.4 | 3.25 | 13 | 13 | 26 | 52 |
| Test II | Day 2 | EU/ml | n.d | n.d | n.d | n.d | n.d | 1280 | 5120 | 5120 | 5120 |
| Tier 3006 | | pg/ml | n.d | n.d | n.d | n.d | n.d | 26 | 104 | 104 | 104 |
| fresh | Day 4 | EU/ml | n.d. | n.d. | n.d. | n.d. | n.d. | 2560 | 10240 | 10240 | 5120 |
| | | pg/ml | n.d. | n.d. | n.d. | n.d. | n.d. | 52 | 208 | 208 | 104 |
| | Day 6 | EU/ml | n.d. | n.d. | n.d. | n.d. | n.d. | 1280 | 2560 | 5120 | 5120 |
| | | pg/ml | n.d. | n.d. | n.d. | n.d. | n.d. | 26 | 52 | 104 | 104 |
| Test III | Day 2 | EU/ml | n.d | n.d | n.d | n.d | n.d | 2560 | 5120 | 10240 | 10240 |
| Tier 2107 | | pg/ml | n.d | n.d | n.d | n.d | n.d | 52 | 104 | 208 | 208 |
| fresh | Day 4 | EU/ml | n.d. | n.d. | n.d. | n.d. | n.d. | 1280 | 5120 | 5120 | 10240 |
| | | pg/ml | n.d. | n.d. | n.d. | n.d. | n.d. | 26 | 104 | 104 | 208 |
| | Day 6 | EU/ml | n.d. | n.d. | n.d. | n.d. | n.d. | 1280 | 2560 | 5120 | 5120 |
| | | pg/ml | n.d. | n.d. | n.d. | n.d. | n.d. | 26 | 52 | 104 | 104 |
| Test IV | Day 2 | EU/ml | n.d | n.d | n.d | n.d | n.d | 160 | 640 | 320 | 160 |
| Tier 2008 | | pg/ml | n.d | n.d | n.d | n.d | n.d | 3.25 | 13 | 6.5 | 3.25 |
| Cryo | Day 4 | EU/ml | n.d. | n.d. | n.d. | n.d. | n.d. | 80 | 640 | 640 | 320 |
| | | pg/ml | n.d. | n.d. | n.d. | n.d. | n.d. | 1.6 | 13 | 13 | 6.5 |
| | Day 6 | EU/ml | n.d. | n.d. | n.d. | n.d. | n.d. | 40 | 640 | 640 | 320 |
| | | pg/ml | n.d. | n.d. | n.d. | n.d. | n.d. | 0.8 | 13 | 13 | 6.5 |

| | | | Concentration μg/ml | | | | | | |
|---|---|---|---|---|---|---|---|---|---|
| | | | IFN | | | | | | |
| Pig | | | 0.2 | 0.1 | 0.01 | — | — | — | — |
| Seq No 2-F | | | 0.2 | 0.1 | 0.05 | 0.025 | 0.012 | 0.006 | 0.003 |
| Test I | Day 2 | EU/ml | 2560 | 2560 | 320 | n.d | n.d | n.d | n.d |
| Tier 2008 | | pg/ml | 52 | 52 | 6.5 | n.d | n.d | n.d | n.d |
| fesh | Day 4 | EU/ml | 2560 | 2560 | 320 | n.d. | n.d. | n.d. | n.d. |
| | | pg/ml | 52 | 52 | 6.5 | n.d. | n.d. | n.d. | n.d. |
| | Day 6 | EU/ml | 2560 | 2560 | 1280 | n.d. | n.d. | n.d. | n.d. |
| | | pg/ml | 52 | 52 | 26 | n.d. | n.d. | n.d. | n.d. |
| Test II | Day 2 | EU/ml | 2560 | 1280 | 640 | 320 | 320 | 160 | 80 |
| Tier 3006 | | pg/ml | 52 | 26 | 13 | 6.5 | 6.5 | 3.25 | 1.6 |
| fresh | Day 4 | EU/ml | 5120 | 2560 | 1280 | 640 | 320 | 160 | 120 |
| | | pg/ml | 104 | 52 | 26 | 13 | 6.5 | 3.25 | 2.42 |
| | Day 6 | EU/ml | 5120 | 2560 | 1280 | 640 | 320 | 160 | 80 |
| | | pg/ml | 52 | 52 | 26 | 13 | 6.5 | 3.25 | 1.6 |
| Test III | Day 2 | EU/ml | 10240 | 5120 | 2560 | 960 | 320 | 160 | 40 |
| Tier 2107 | | pg/ml | 208 | 104 | 52 | 19.5 | 6.5 | 3.25 | 0.8 |
| fresh | Day 4 | EU/ml | 10240 | 10240 | 5120 | 1920 | 480 | 160 | 80 |
| | | pg/ml | 208 | 208 | 104 | 39 | 9.75 | 3.25 | 1.6 |
| | Day 6 | EU/ml | 2560 | 2560 | 960 | 640 | 320 | 160 | 80 |
| | | pg/ml | 52 | 52 | 19.5 | 13 | 6.5 | 3.25 | 1.6 |
| Test IV | Day 2 | EU/ml | 160 | 80 | 40 | 20 | 0 | 0 | 0 |
| Tier 2008 | | pg/ml | 3.25 | 1.6 | 0.8 | 0.4 | 0 | 0 | 0 |
| Cryo | Day 4 | EU/ml | 160 | 80 | 40 | 20 | 0 | 0 | 0 |
| | | pg/ml | 3.25 | 1.6 | 0.8 | 0.4 | 0 | 0 | 0 |
| | Day 6 | EU/ml | 160 | 160 | 80 | 40 | 20 | 0 | 0 |
| | | pg/ml | 3.25 | 3.25 | 1.6 | 0.8 | 0.4 | 0 | 0 |

To compare the stimulation behavior of freshly isolated PMBC against cryopreserved cells another VSV assay (Test IV) was performed with frozen PBMC from pigs. For this, the fresh and cryopreserved cells of the pig no. 2008 were used for a direct comparison.

### Results

Seq No 2-F was found to be a highly effective stimulator of interferon release in PMBCs. Additional costimuli did not result in a further increase in interferon release as no additive effect was detectable. The biological activity of the released interferon was described in terms of an experimental unit (EU). An experimental unit was defined by a 50% CPE. The calculation of the EU units in ml was carried out by the formula: 2x×10=EU/ml.

Toxicity/Intolerance

Referring to Table 1, the highest concentrations of SEQ ID NO. 2, 100−25 μg/ml, resulted in immunomodulator-related intolerances, and a lack of or reduced interferon release. Microscopic analysis of the cells detected morphological changes suggesting toxicity. Assays for interferon release were also performed with lower concentrations of SEQ ID NO. 2 ranging from 3 μg/ml to 0.003 ug/ml. None of the lower concentrations exhibited toxicity similar to that seen in the higher concentration.

Interferon Release

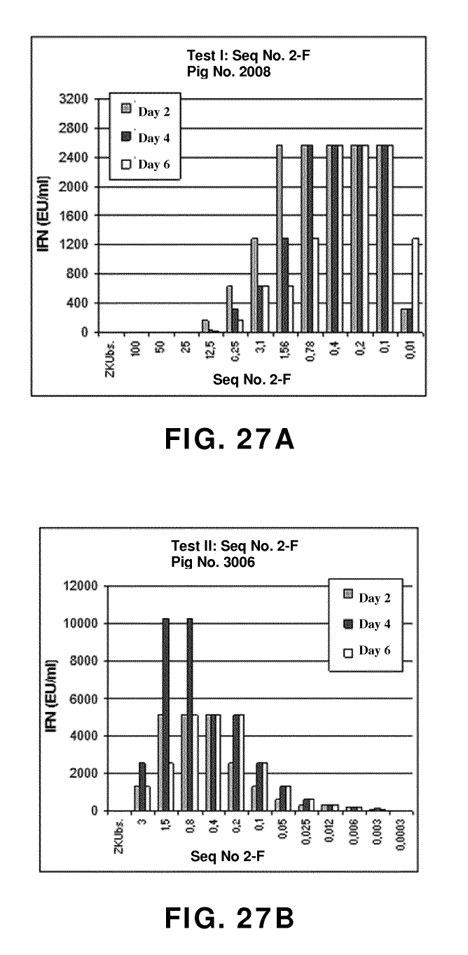

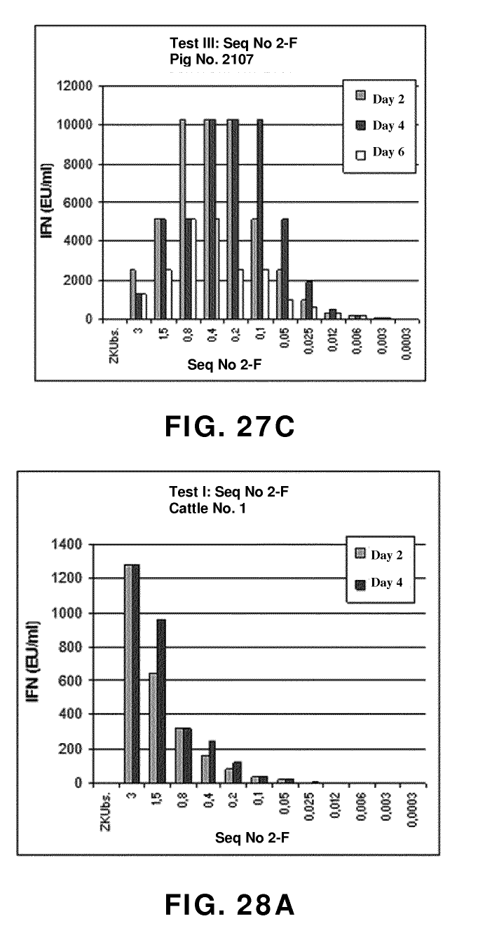

Administration of Seq No 2-F resulted in interferon release. In all 3 pigs (tests I, II, and III), Seq No 2-F induced IFN release in a dose-dependent manner (FIGS. 27A-C). The amount of IFN released differed individually in the respective animals.

The cryopreserved PBMCs treated with Seq No 2-F released IFN; however, the average amount of IFN released was approximately 4 times lower than the IFN released from the freshly isolated cells (see Table 1, Test I and Test IV).

### Example 8: Ex Vivo Induction of IFN Release in Cattle

The purpose of this study was to determine the effect of formulated Seq No 2 on the interferon production in peripheral blood mononuclear cells (PMBCs) isolated from cattle.

Vesicular stomatitis virus (VSV) cytolytic assay were employed to detect type 1 interferon expression. The VSV assays (Tests I, II, and III) were performed on PMBCs isolated from three separate cattle. The PMBCs were treated with formulated Seq No 2-F both with and without costimulus (UV-inactivated herpes virus). Referring to Table 2, the analysis was carried out at differing concentrations of Seq No 2-F (3 μg/ml to 0.003 g/ml) over the course of 2 and 4 days.

TABLE 2
Interferon release from bovine PMBCs

| | | | Concentration (μg/ml) | | | | | | | | | | | |
|---|---|---|---|---|---|---|---|---|---|---|---|---|---|---|
| | | | IFN | | | | | | | | | | | |
| Cattle | | | | | | | | | | | | | | 0.0003 |
| Seq No 2-F | | | 3 | 1.5 | 0.8 | 0.4 | 0.2 | 0.1 | 0.05 | 0.025 | 0.012 | 0.006 | 0.003 | (0.3 ng/ml) |
| Test I | Day 2 | EU/ml | 1280 | 640 | 320 | 160 | 80 | 40 | 20 | 0 | 0 | 0 | 0 | 0 |
| No. 1 | | pg/ml | 26 | 13 | 6.5 | 3.25 | 1.6 | 0.8 | 0.4 | 0 | 0 | 0 | 0 | 0 |
| fresh | Day 4 | EU/ml | 1280 | 960 | 320 | 240 | 120 | 40 | 20 | 10 | 0 | 0 | 0 | 0 |
| | | pg/ml | 26 | 19.5 | 6.5 | 4.87 | 2.42 | 0.8 | 0.4 | 0.2 | 0 | 0 | 0 | 0 |
| Test II | Day 2 | EU/ml | 160 | 160 | 240 | 80 | 40 | 20 | 0 | 0 | 0 | 0 | 0 | 0 |
| No. 2 | | pg/ml | 3.25 | 3.25 | 4.87 | 1.6 | 0.8 | 0.4 | 0 | 0 | 0 | 0 | 0 | 0 |
| fresh | Day 4 | EU/ml | 320 | 160 | 160 | 120 | 60 | 20 | 0 | 0 | 0 | 0 | 0 | 0 |
| | | pg/ml | 6.5 | 3.25 | 3.25 | 2.42 | 1.2 | 0.4 | 0 | 0 | 0 | 0 | 0 | 0 |
| Test III | Day 2 | EU/ml | 320 | 160 | 80 | 80 | 40 | 40 | 10 | 0 | 0 | 0 | 0 | 0 |
| No. 3 | | pg/ml | 6.5 | 3.25 | 1.6 | 1.6 | 0.8 | 0.8 | 0.2 | 0 | 0 | 0 | 0 | 0 |
| fresh | Day 4 | EU/ml | 480 | 240 | 80 | 80 | 80 | 60 | 40 | 0 | 0 | 0 | 0 | 0 |
| | | pg/ml | 9.75 | 4.87 | 1.6 | 1.6 | 1.6 | 1.2 | 0.8 | 0 | 0 | 0 | 0 | 0 |
| Test IV | Day 2 | EU/ml | n.d | n.d | n.d | n.d | n.d | n.d | n.d | n.d | n.d | n.d | n.d | n.d |
| No. 1 | | pg/ml | n.d | n.d | n.d | n.d | n.d | n.d | n.d | n.d | n.d | n.d | n.d | n.d |
| Cryo | Day 4 | EU/ml | 240 | 80 | 40 | 10 | 0 | 0 | 0 | 0 | 0 | 0 | 0 | 0 |
| | | pg/ml | 4.87 | 1.6 | 0.8 | 0.2 | 0 | 0 | 0 | 0 | 0 | 0 | 0 | 0 |

To compare the stimulation behavior of freshly isolated PMBC against cryopreserved cells, a VSV assay (Test IV) was performed with frozen PBMC from the cow used in Test I, so that a direct comparison between the cryopreserved cells and the cells freshly isolated from the cow.

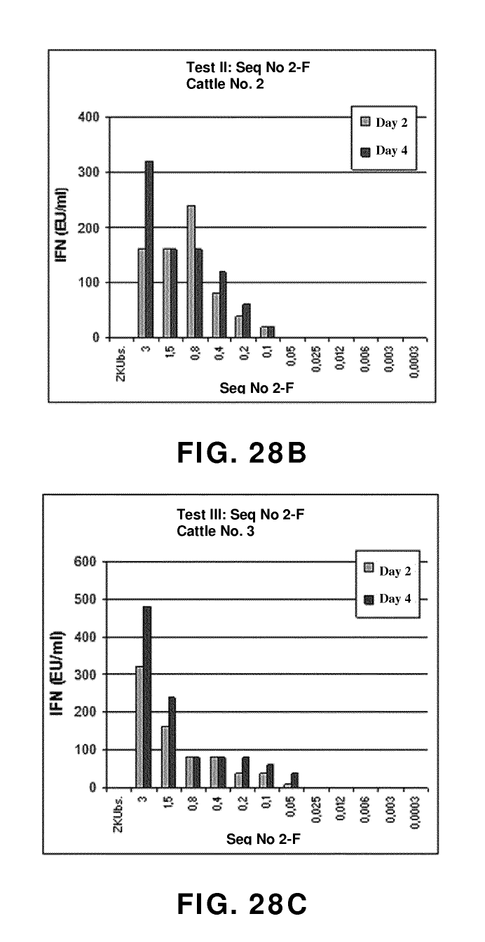

Seq No 2-F was found to be a highly effective stimulator of interferon release in PMBCs. Additional co-stimuli did not result in a further increase in interferon release as no additive effect was detectable. Administration of Seq No 2-F resulted in IFN release. In all 3 cows (Tests I, II, and III), Seq No 2-F-induced IFN release in a dose-dependent manner as illustrated in FIGS. 28A-C. The amount of IFN released differed individually in the respective animals. The cryopreserved PBMCs treated with Seq No 2-F released IFN; however, the average amount of IFN released was approximately 5 times was lower than the IFN released from the freshly isolated cells (see Table 2, Test I and Test IV).

### Example 9: Cross Over Study of Immunomodulator Dose and Administration Route

A cross over study was performed to compare the effects immunomodulator dosage amounts and methods of administration had on cytokine expression in pigs.

The Seq No 2-F was given once weekly (7 day interval) either subcutaneously in a high or low dose or intramuscularly in a high or low dose for a period of 4 weeks. In two pigs the test substance was administered slowly intravenously in a high or low dose in the fifth week. The schedule according to a Latin Square design is depicted in Table 3.

Pigs were treated with either a high or a low dose of Seq No 2-F, which was administered either subcutaneously or intramuscularly. At 1 hour before treatment and 2 hours, 6 hours, 24 hours, and 48 hours after treatment serum and whole blood cells were collected to investigate serum cytokine levels and mRNA expression of cytokines in circulating blood cells. The treatment was repeated four times in alternating animals as shown in Table 3. The intervals between treatments were 7 days.

TABLE 3
Schedule of allocation of treatment (doses/route of administration)

| | Animal | | | | | |
|---|---|---|---|---|---|---|
| Treatment (date) | 8053 | 7934 | 7840 | 7868 | 7773 | 7675 |
| 1 (4 Nov. 2008) | sc low | sc low | im high | im low | im low | sc high |
| 2 (11 Nov. 2008) | sc high | im high | im low | sc low | sc high | im high |
| 3 (18 Nov. 2008) | im low | im low | sc high | im high | sc low | sc low |
| 4 (25 Nov. 2008) | im high | sc high | sc low | sc high | im high | im low |

sc (subcutaneous)
im (intramuscular)

Two days after the last test product administration pigs were euthanized and injection sites during gross pathology examined. Tissue specimens of injection sites were sampled for histological examination.

### Animals

Pigs derived from a high health swine herd, (VOF G. v. Beek, Runderweg 10, 8219 Lelystad), which is free of porcine reproductive and respiratory syndrome virus, post weaning multisystemic wasting syndrome, Mycoplasma hyopneumoniae, Actinobacillus pleuropneumoniae. Veterinary inspection at arrival revealed that pigs were free of pneumonia, diarrhea, inflammatory changes of the skin or the tail, or other signs of sickness.

### Dosing Regimen, Frequency, and Duration

The Seq No 2-F was administered in a single shot according to the treatment schedule. The high dose for intramuscular or subcutaneous administration was 205 μg and the low dose was 20 μg in a volume of 2 ml. The high dose for intravenous administration was is 50 μg in 5 ml, and the low dose is 10 μg in a volume of 5 ml.

### Observations

Body temperatures were recorded on day −1 before immunomodulator administration and 6, 24, and 48 hours after administration. Body weight was recorded prior to surgery on day −7 and prior to necropsy on day 23 (four pigs) or 30 (two pigs), respectively.

### Analytical Methods

Hematology: WBC count, differential blood cell composition (lymphocytes, mononuclear cells and granulocytes), red blood cell count, hemoglobulin, hematocrit, mean corpuscular value were assessed by standard laboratory techniques.

Serum cytokine analysis: The following porcine cytokines were measured by protein array technology (Pierce, Search Light®): IL-1b, IL-2, IL-4, IL-6, IL-8, IL-10, IL-12, IFNγ, TNFa. Pierce SearchLight Proteome Arrays are multiplexed assays that measure of up to 16 proteins per well. SearchLight Arrays are produced by spotting different monoclonal antibodies into each well of a 96-well plate.

Cytokine mRNA analysis: Expression of IL-1, IL-2, IL-6, IL-10, IL-12 was assessed by qPCR technology (Applied Biosystems). Total RNA was isolated using a TRIzol reagent (Invitrogen, Breda, The Netherlands) according to the manufacturer's instructions. The remaining RNA was dissolved in 50 μl of RNase free water and was quantified spectrophotometrically using Nanodrop ND-1000 (Isogen Life Sciences, IJsselstein, The Netherlands). cDNA synthesis and Q-PCR conditions were performed according to standard lab procedure. Information about the primers used is depicted in Table 4. To reduce amplification of trace amounts of genomic DNA, the primers were positioned in different exons. Calculations to estimate the expression stability and pair wise variation were performed with the freely available GeNorm program (http://medgen.ugent.be/jvdsomp/genorm).

All cytokine mRNA expressions were compared to the expression of Actin B (ACTB) and expressed as relative amounts by calculating the amount of cytokine mRNA/amount of ACTB mRNA.

TABLE 4
Cytokine mRNA analysis

| | | | | Product | |
|---|---|---|---|---|---|
| Porcine | Accession | Forward primer | Reverse primer | length | Ta |
| cytokin | number | 5′ → 3′ | 5′ → 3′ | (bp) | (C. °) |
| IL-1β | NM_001005149 | SEQ ID NO: 5 | SEQ ID NO: 6 | 70 | 59 |
| IL-2 | NM_213861 | SEQ ID NO: 7 | SEQ ID NO: 8 | 70 | 59 |
| IL-6 | AF518322 | SEQ ID NO: 9 | SEQ ID NO: 10 | 68 | 59 |
| IL-10 | NM_214041 | SEQ ID NO: 11 | SEQ ID NO: 12 | 69 | 59 |
| IL-12p40 | AY388985 | SEQ ID NO: 13 | SEQ ID NO: 14 | 88 | 59 |

### Statistical Analysis

Descriptive analyses were performed. A preliminary inferential statistical analysis of cytokine mRNA expression was performed by conducting a two-way ANOVA analysis (time; treatment). All statistical data were analyzed with GraphPad Prism, version 4 for Windows (GraphPad Software, San Diego, Ca, USA).

### Results

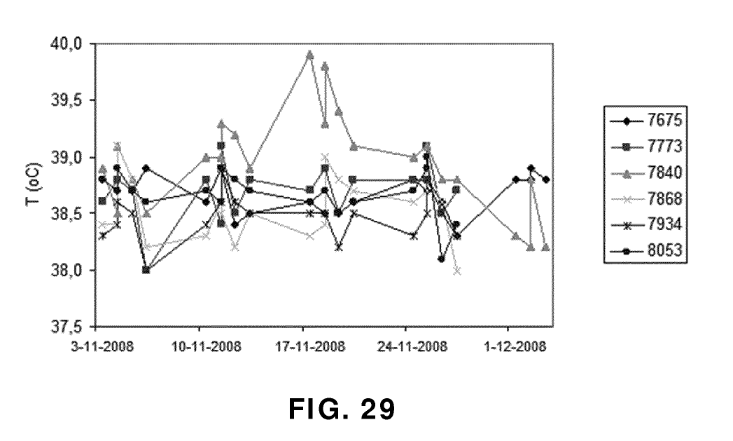

No clinical reactions were observed after administration of the test substance immunomodulator. Rectal body temperatures are depicted in FIG. 29. Body temperatures were within the normal temperature range of pigs. One animal (no. 7840) exhibited an increase to a near febrile temperature on the third treatment day, but the temperature increase was not treatment-related.

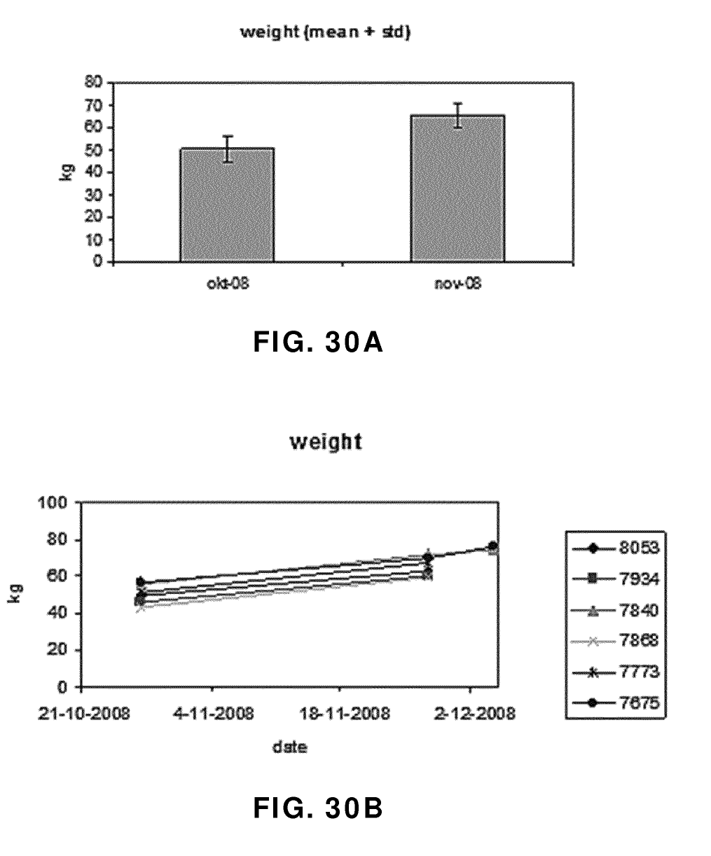

No difference was seen between pigs in regard to body weight increase during the test period (FIGS. 30A and 30B).

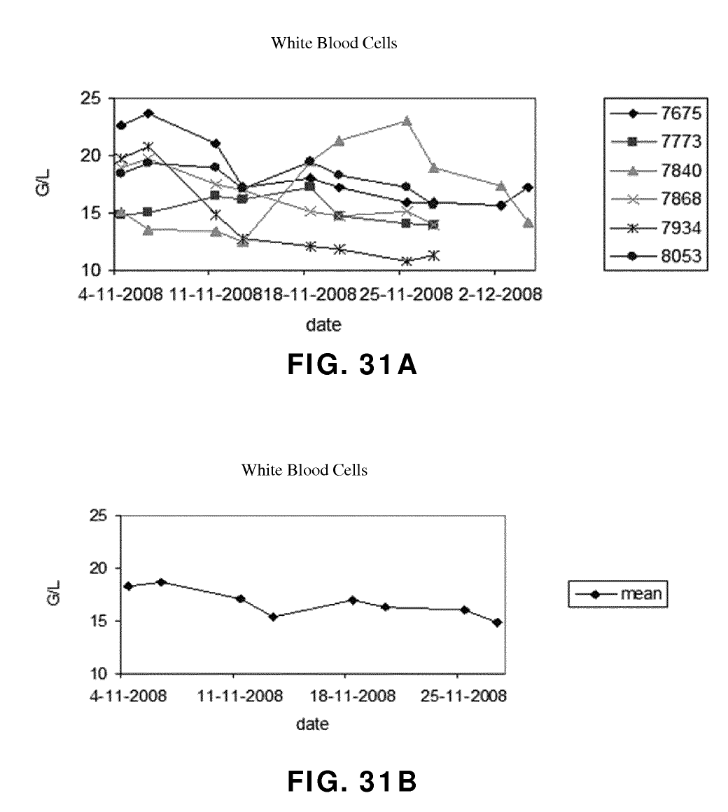

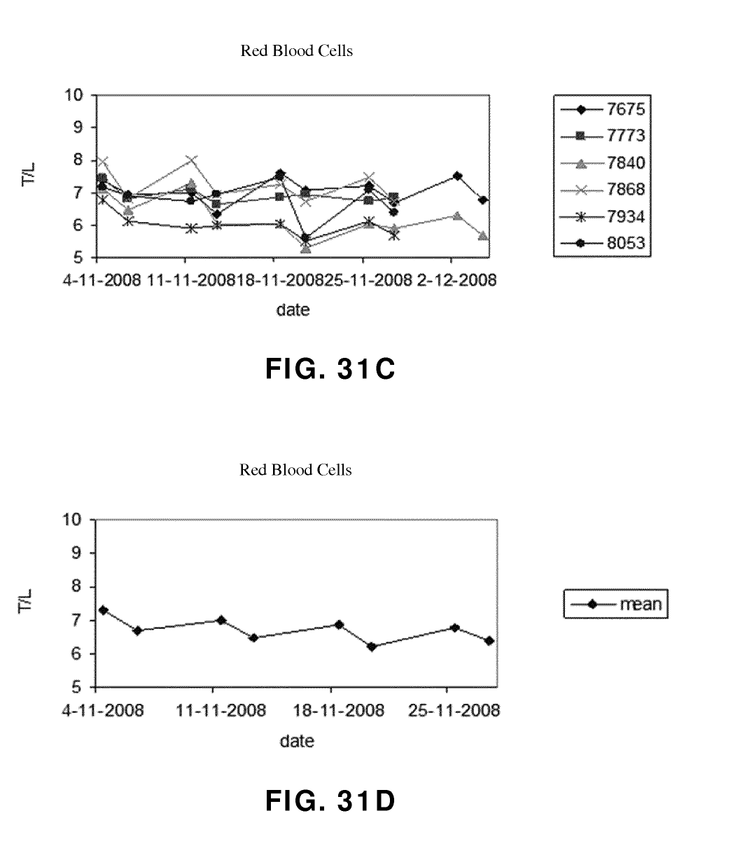

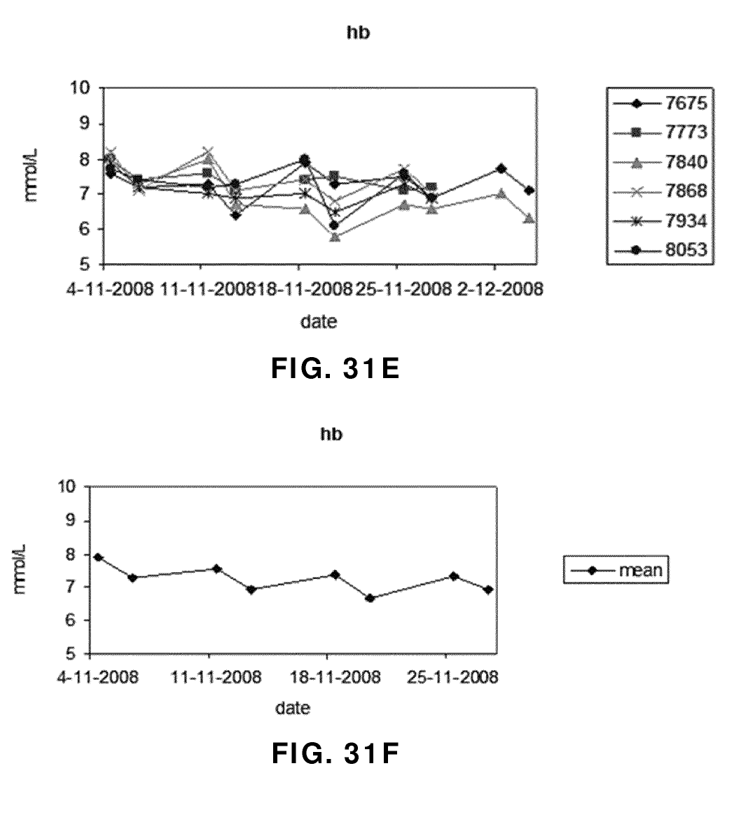

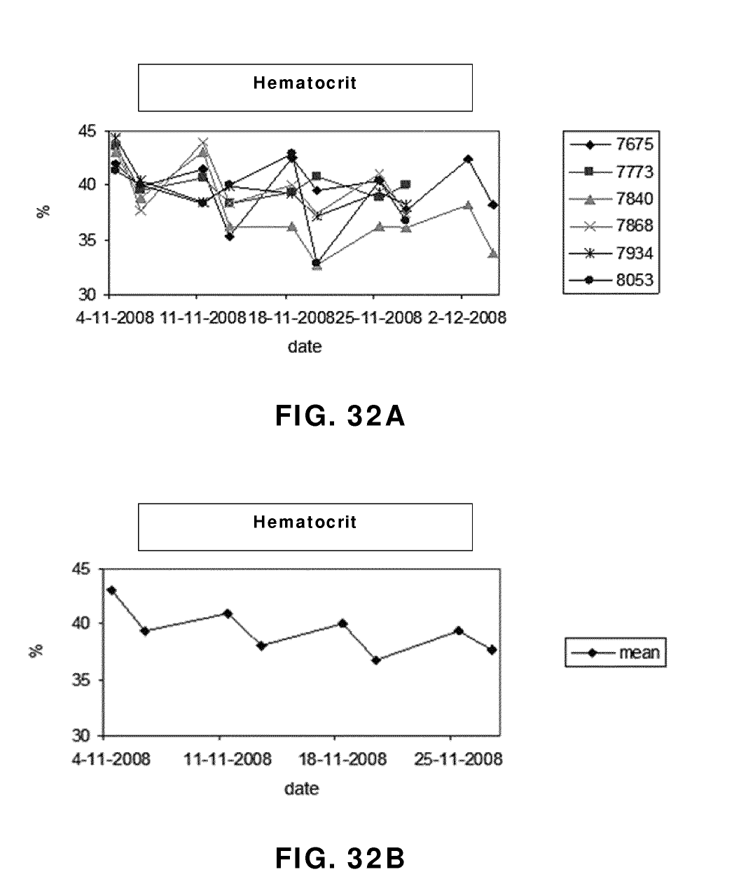

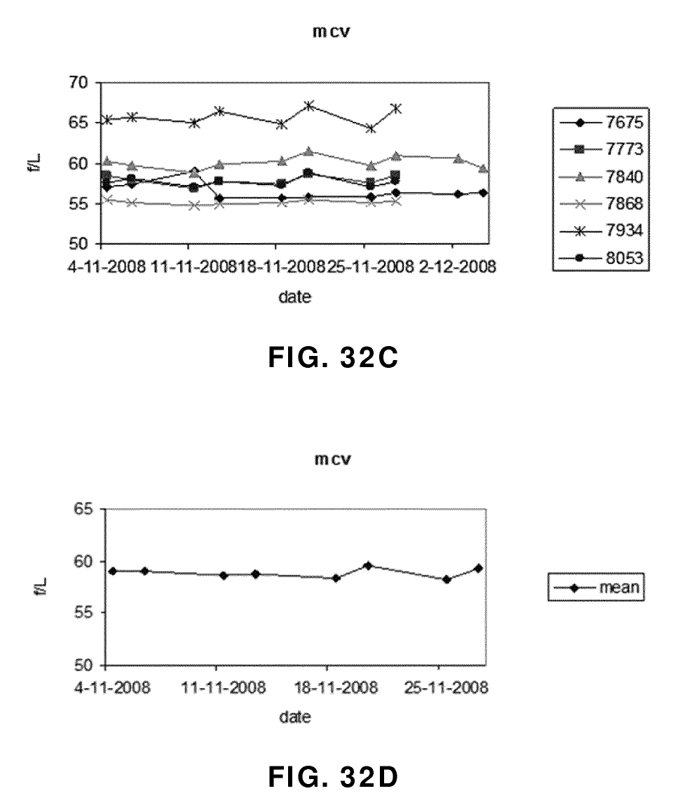

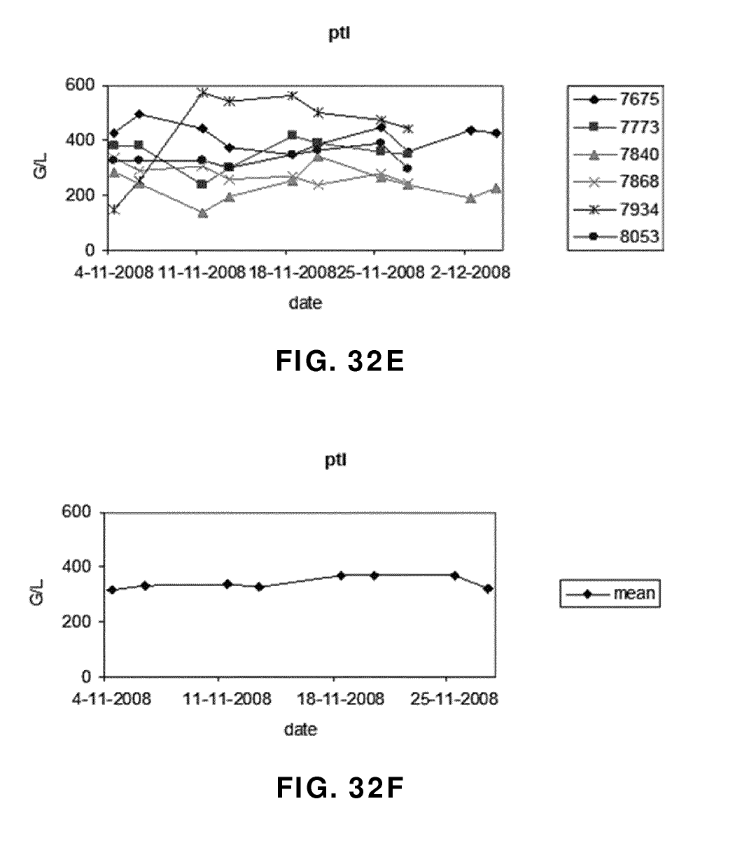

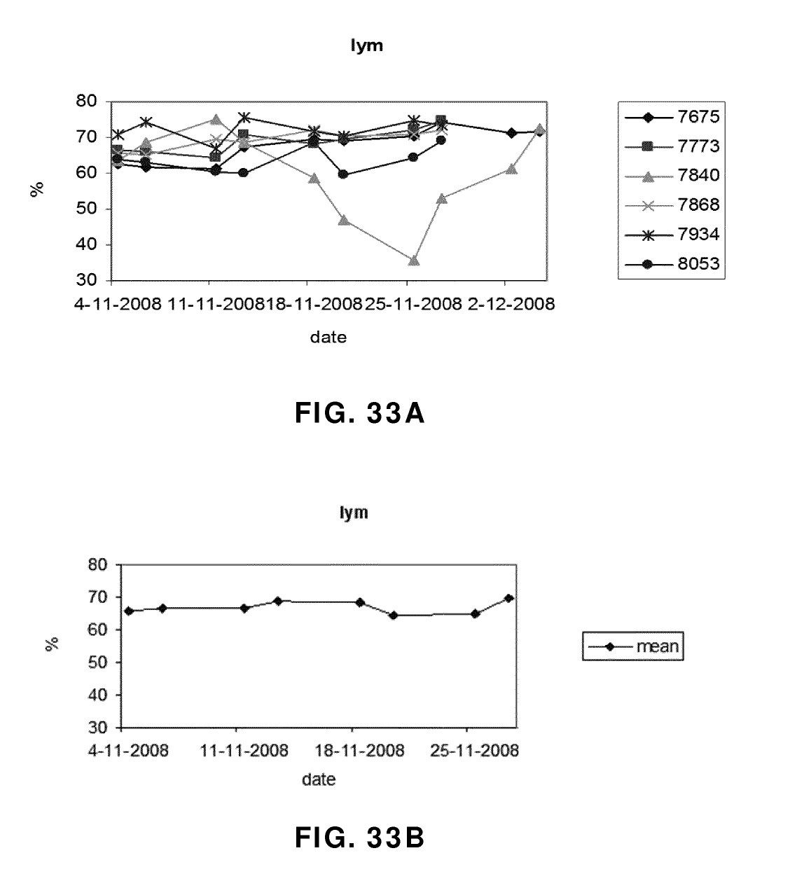

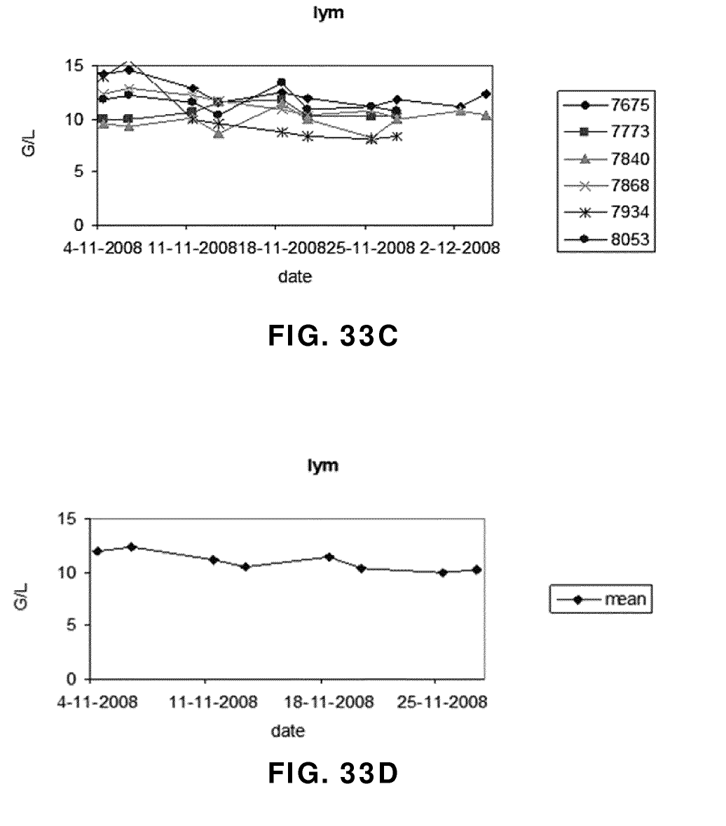

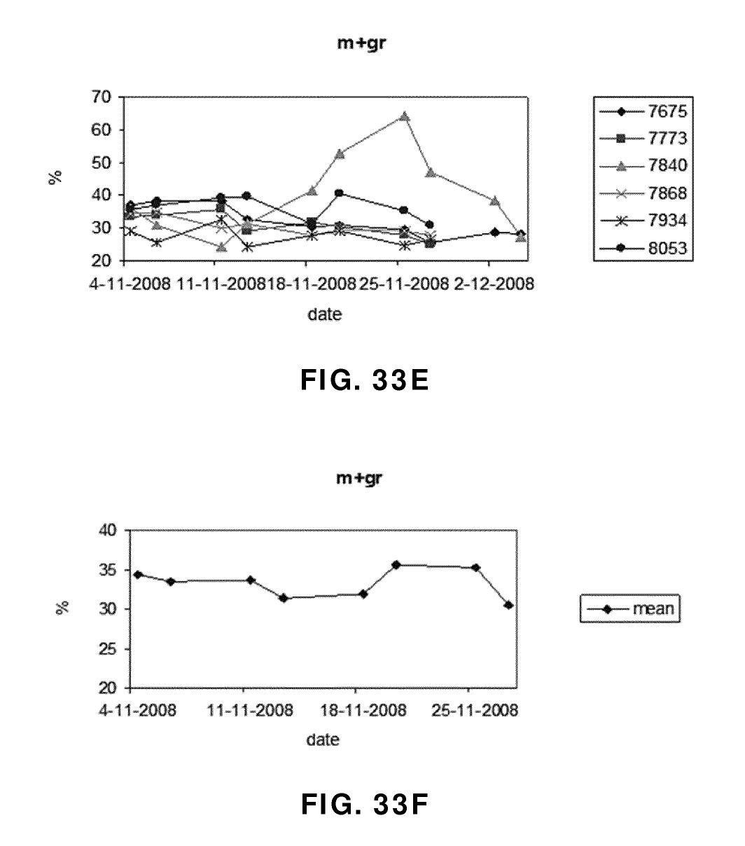

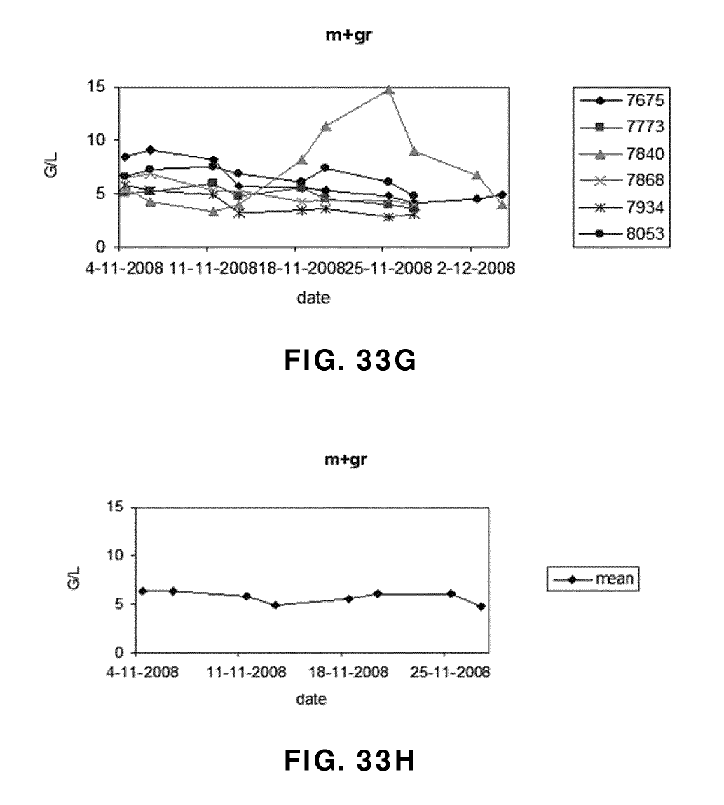

Hematology data are presented in FIGS. 31A-F, 32A-F, and 33A-H. Generally, hematological data were before and after treatment found to be in the normal range. Striking was a lowering of the means of red blood cells (RBC), Hemoglobulin (HB) and hematocrit after treatment, however means stayed within the physiological ranges. Mean corpuscular volume (MCV), platelet, lymphocyte, and mononuclear/granulocyte counts remained nearly constant.

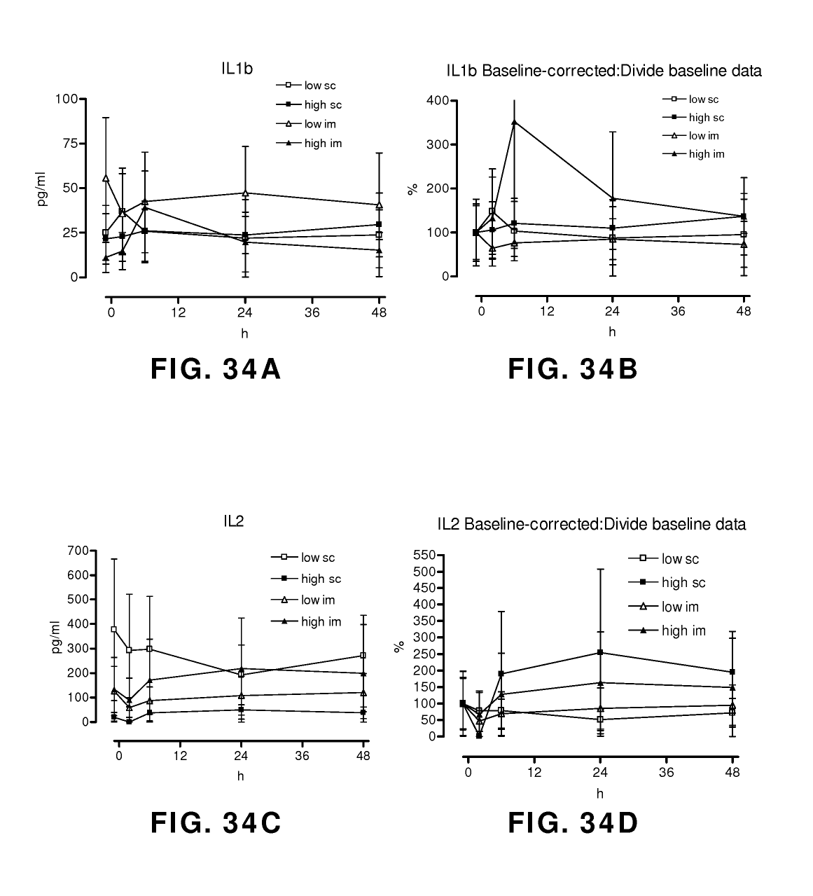

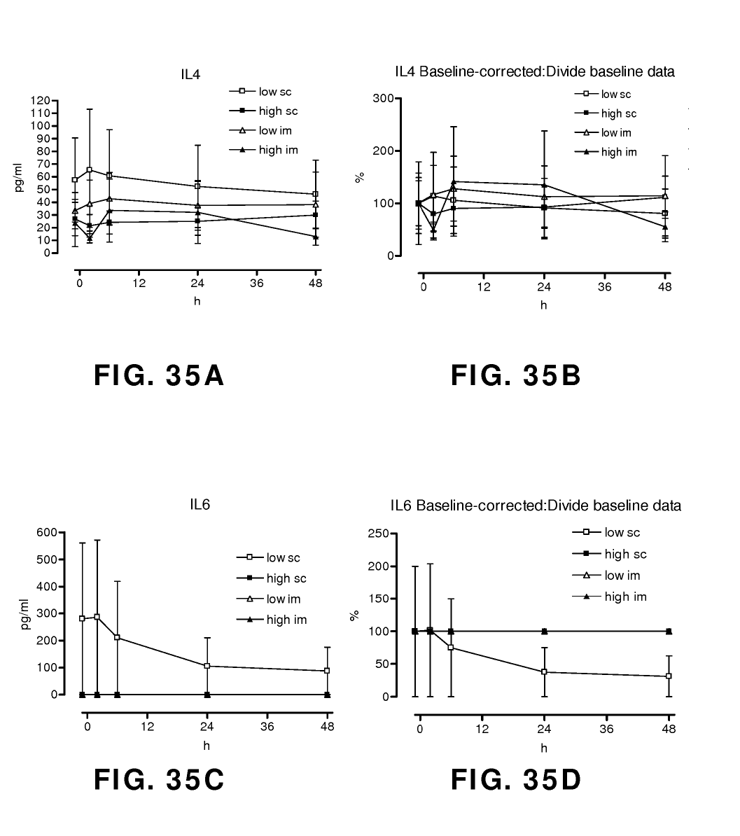

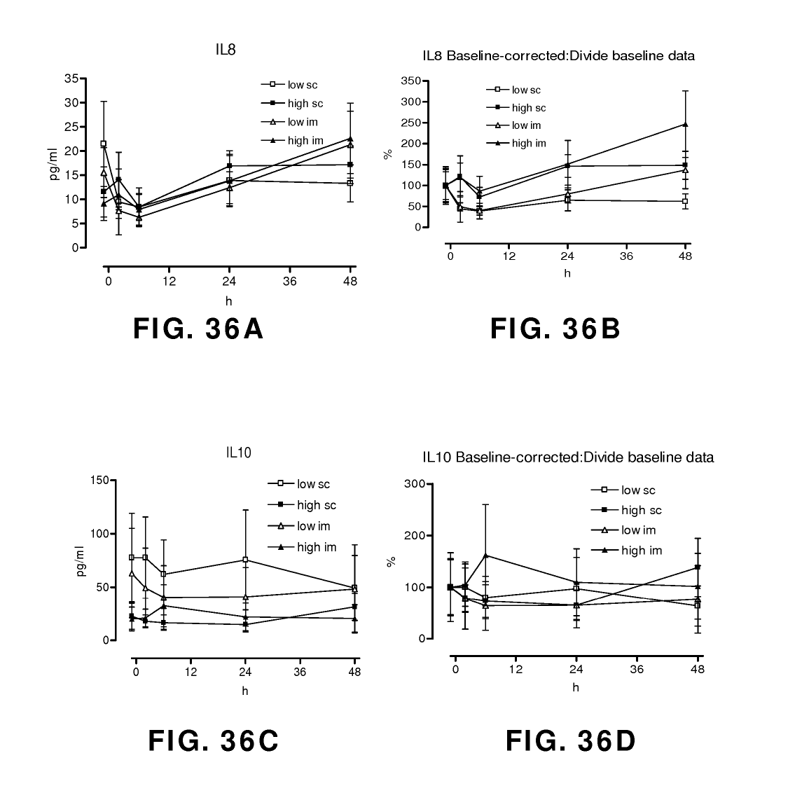

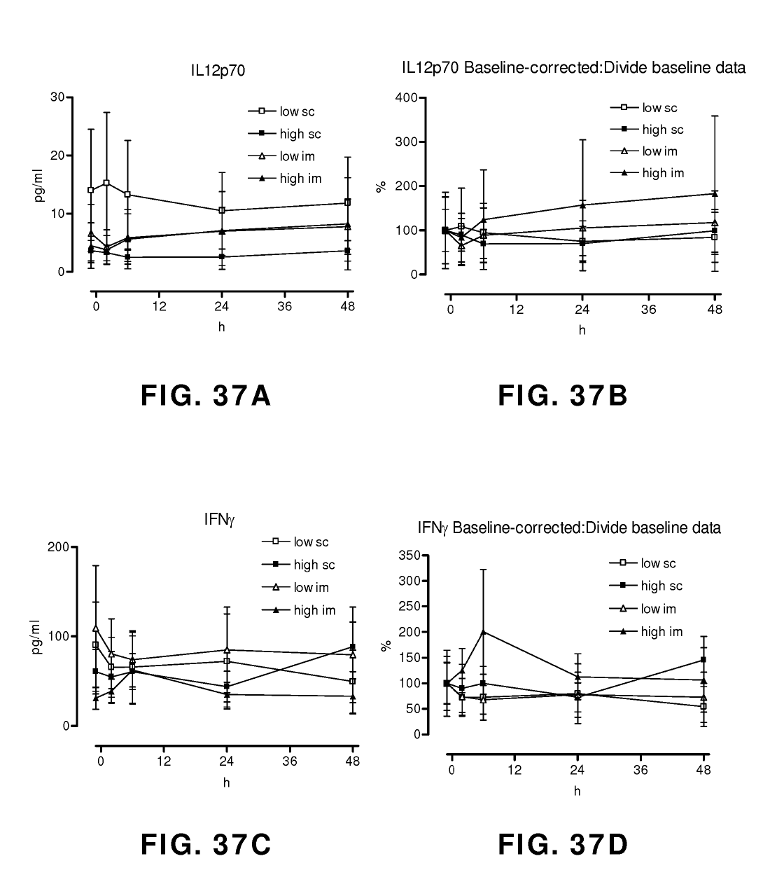

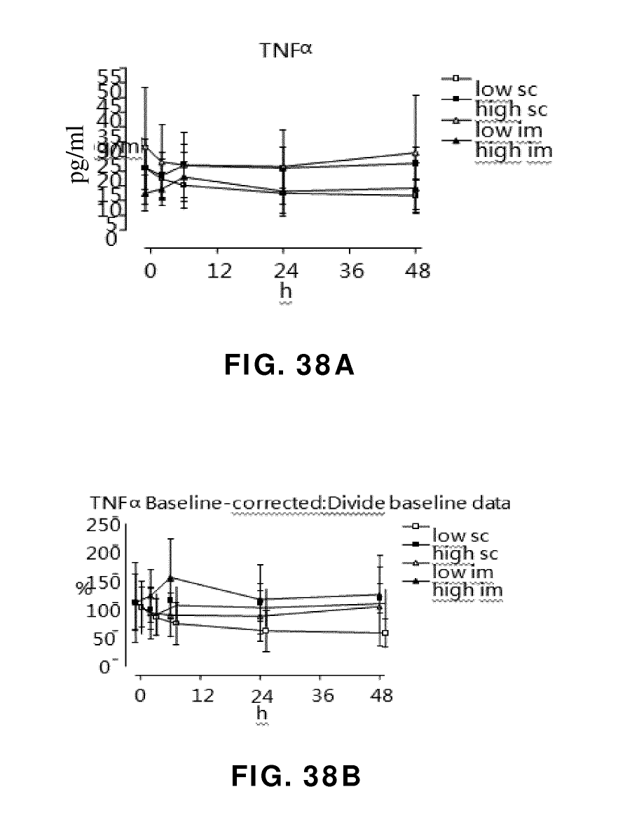

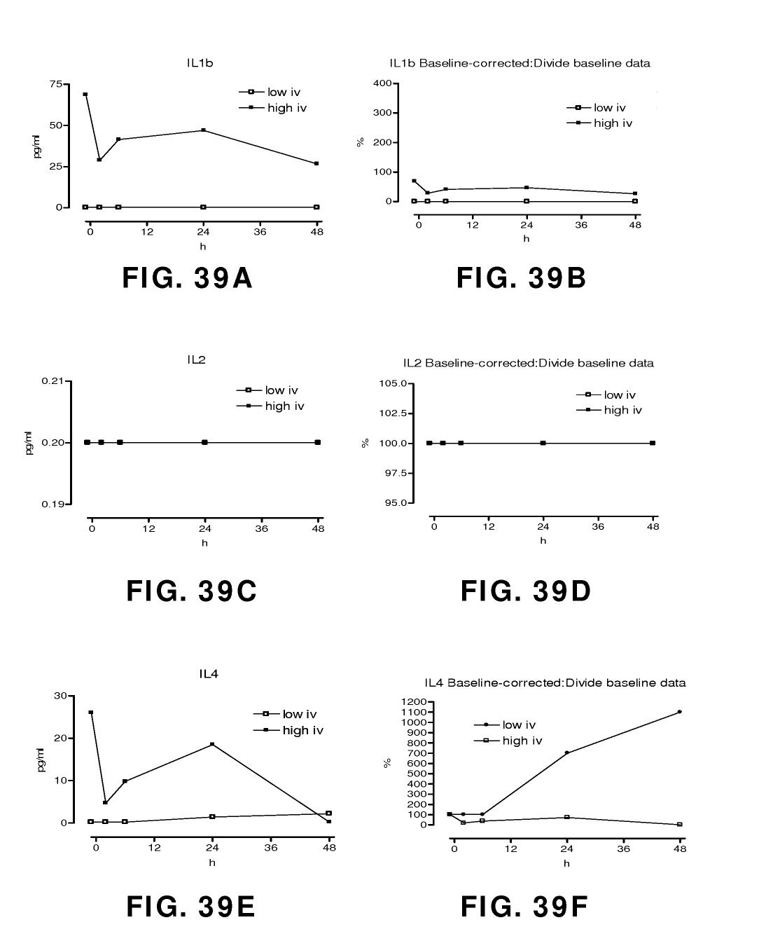

Data from the serum cytokine protein array is shown in Table 5 (from section 12). For calculations of means and graphical demonstration all values “under detection limit” were transformed to the lowest detectable level of 0.2 pg/ml. Certain cytokines were not consistently detected in the serum, i.e. IL-1, IL-4, IL-10, IFNγ and TNF in more than ⅔ of the pigs, whereas IL-2 and IL-12 were only detected in ½ of the pigs and IL-6 was with one exception (pig no. 7840 on treatment day 4) not detected. The results are graphically demonstrated in FIGS. 34A-D, 35A-D, 36A-D, 37A-D, and 38A and B in regard to repeated measurements (high dose, low dose, intramuscular, subcutaneous) and in FIGS. 39A-F, 40A-F, and 41A-H in regard to the single measurements after intravenous administration. The graphs show the mean content of the different cytokines and also the proportional change of cytokine content after administration of the test substance. No striking changes compared to pre-treatment measurements were observed for IL-4, IL-6, IL-8, IL-10, IL-12, IFNγ and TNF. A more than two fold mean increase was observed for IL-1 after intramuscular administration of the high dose of the test substance immunomodulator and for IL-2 after intramuscular and subcutaneous treatment with a high dose of the test substance immunomodulator.

TABLE 5
Serum cytokine content before and after treatment measured by porcine cytokine protein array

| animal nr | | animal | cytokine | | IL1b | IL2 | IL4 | IL6 | IL8 | IL10 | IL12p70 | IFNg | TNFa |
|---|---|---|---|---|---|---|---|---|---|---|---|---|---|
| in study plan | treatment | ID | date of admin. | h.p.t. | pg/ml | pg/ml | pg/ml | pg/ml | pg/ml | pg/ml | pg/ml | pg/ml | pg/ml |
| 6 | sc-high | 7675 | 4 Nov. 2008 | −1 | 81.8 | 0.2 | 25 | 0.2 | 7.4 | 9.7 | 4.2 | 134.1 | 35.9 |
| 6 | sc-high | 7675 | 4 Nov. 2008 | 2 | 72 | 0.2 | 27.2 | 0.2 | 6.2 | 21.1 | 5.4 | 182.5 | 32.8 |
| 6 | sc-high | 7675 | 4 Nov. 2008 | 6 | 70.9 | 0.2 | 20.2 | 0.2 | 5.3 | 4.8 | 2.8 | 126.6 | 28.7 |
| 6 | sc-high | 7675 | 4 Nov. 2008 | 24 | 50.6 | 0.2 | 18.6 | 0.2 | 8.9 | 0.2 | 1.5 | 86.5 | 22.5 |
| 6 | sc-high | 7675 | 4 Nov. 2008 | 48 | 27.8 | 0.2 | 10.9 | 0.2 | 4.1 | 0.2 | 0.6 | 54.9 | 21.6 |
| 5 | sc-high | 7773 | 11 Nov. 2008 | −1 | 0.2 | 0.2 | 4.2 | 0.2 | 0.2 | 17.6 | 0.2 | 19.6 | 3.1 |
| 5 | sc-high | 7773 | 11 Nov. 2008 | 2 | 0.2 | 0.2 | 2.8 | 0.2 | 3.2 | 24.4 | 0.2 | 12.4 | 4.2 |
| 5 | sc-high | 7773 | 11 Nov. 2008 | 6 | 39.8 | 0.2 | 11.5 | 0.2 | 4.6 | 25.6 | 0.2 | 60.4 | 15.8 |
| 5 | sc-high | 7773 | 11 Nov. 2008 | 24 | 36.5 | 0.2 | 14 | 0.2 | 13 | 24 | 0.2 | 52.2 | 19.2 |
| 5 | sc-high | 7773 | 11 Nov. 2008 | 48 | 34.4 | 103.7 | 35.6 | 0.2 | 9.5 | 84.9 | 0.2 | 195.8 | 27.3 |
| 3 | sc-high | 7840 | 18 Nov. 2008 | −1 | 0.2 | 116.4 | 83.3 | 0.2 | 34.2 | 79.8 | 10.6 | 142.3 | 59 |
| 3 | sc-high | 7840 | 18 Nov. 2008 | 2 | 3.7 | 0.2 | 47.2 | 0.2 | 1.2 | 26.6 | 4.4 | 61.2 | 38.1 |
| 3 | sc-high | 7840 | 18 Nov. 2008 | 6 | 0.9 | 0.2 | 58 | 0.2 | 10.2 | 43.3 | 7.3 | 105.4 | 38 |
| 3 | sc-high | 7840 | 18 Nov. 2008 | 24 | 0.2 | 297.9 | 72.6 | 0.2 | 11.1 | 37.2 | 9.3 | 98.4 | 32.9 |
| 3 | sc-high | 7840 | 18 Nov. 2008 | 48 | 16 | 124 | 76.2 | 0.2 | 16.8 | 37.8 | 11.2 | 56.6 | 25.2 |
| 4 | sc-high | 7868 | 25 Nov. 2008 | −1 | 1.2 | 0.2 | 4.4 | 0.2 | 16.3 | 0.2 | 0.2 | 14.5 | 0.2 |
| 4 | sc-high | 7868 | 25 Nov. 2008 | 2 | 0.2 | 0.2 | 5.5 | 0.2 | 25 | 0.2 | 1.2 | 0.2 | 0.2 |
| 4 | sc-high | 7868 | 25 Nov. 2008 | 6 | 0.2 | 0.2 | 11.8 | 0.2 | 18.9 | 0.2 | 0.2 | 0.2 | 0.2 |
| 4 | sc-high | 7868 | 25 Nov. 2008 | 24 | 2.3 | 0.2 | 6.4 | 0.2 | 16.5 | 0.2 | 1 | 2.8 | 5.7 |
| 4 | sc-high | 7868 | 25 Nov. 2008 | 48 | 60.8 | 0.2 | 21.7 | 0.2 | 22.5 | 26.8 | 4.7 | 94.3 | 20.6 |
| 2 | sc-high | 7934 | 25 Nov. 2008 | −1 | 45.9 | 0.2 | 44.2 | 0.2 | 10.9 | 28.9 | 6.5 | 39.3 | 28.2 |
| 2 | sc-high | 7934 | 25 Nov. 2008 | 2 | 61 | 0.2 | 46.1 | 0.2 | 36.3 | 34.9 | 8.3 | 71.5 | 35.4 |
| 2 | sc-high | 7934 | 25 Nov. 2008 | 6 | 44.8 | 221.8 | 43.5 | 0.2 | 11.2 | 25.8 | 4.5 | 53.1 | 41.7 |
| 2 | sc-high | 7934 | 25 Nov. 2008 | 24 | 52.7 | 0.2 | 38.2 | 0.2 | 28.5 | 27 | 3.2 | 24.2 | 45.8 |
| 2 | sc-high | 7934 | 25 Nov. 2008 | 48 | 38.1 | 0.2 | 35.2 | 0.2 | 19 | 39 | 4.8 | 128.2 | 40.5 |
| 1 | sc-high | 8053 | 11 Nov. 2008 | −1 | 0.2 | 0.2 | 0.2 | 0.2 | 0.3 | 0.2 | 0.2 | 13.9 | 0.2 |
| 1 | sc-high | 8053 | 11 Nov. 2008 | 2 | 0.2 | 0.2 | 0.2 | 0.2 | 12.5 | 0.2 | 0.2 | 0.2 | 0.2 |
| 1 | sc-high | 8053 | 11 Nov. 2008 | 6 | 0.2 | 0.2 | 0.2 | 0.2 | 0.2 | 0.2 | 0.2 | 18.4 | 7 |
| 1 | sc-high | 8053 | 11 Nov. 2008 | 24 | 0.2 | 0.2 | 0.2 | 0.2 | 23.5 | 0.2 | 0.2 | 0.2 | 0.2 |
| 1 | sc-high | 8053 | 11 Nov. 2008 | 48 | 0.2 | 0.2 | 0.2 | 0.2 | 31.1 | 0.2 | 0.2 | 0.2 | 0.2 |
| 6 | sc-low | 7675 | 18 Nov. 2008 | −1 | 0.2 | 0.2 | 2.3 | 0.2 | 44.7 | 0.2 | 0.2 | 0.2 | 6.3 |
| 6 | sc-low | 7675 | 18 Nov. 2008 | 2 | 0.2 | 0.2 | 0.9 | 0.2 | 2.9 | 0.2 | 0.2 | 0.2 | 9 |
| 6 | sc-low | 7675 | 18 Nov. 2008 | 6 | 0.2 | 0.2 | 0.2 | 0.2 | 5.4 | 0.2 | 0.2 | 3.5 | 4.3 |
| 6 | sc-low | 7675 | 18 Nov. 2008 | 24 | 0.2 | 0.2 | 0.2 | 0.2 | 11.8 | 0.2 | 0.2 | 0.2 | 4 |
| 6 | sc-low | 7675 | 18 Nov. 2008 | 48 | 0.2 | 0.2 | 0.2 | 0.2 | 23.2 | 0.2 | 0.2 | 0.2 | 0.2 |
| 5 | sc-low | 7773 | 18 Nov. 2008 | −1 | 9.2 | 111.8 | 18.6 | 0.2 | 21.3 | 83 | 0.2 | 203.1 | 29.6 |
| 5 | sc-low | 7773 | 18 Nov. 2008 | 2 | 1 | 0.2 | 7.5 | 0.2 | 1.4 | 87.5 | 0.2 | 111.4 | 10 |
| 5 | sc-low | 7773 | 18 Nov. 2008 | 6 | 0.2 | 0.2 | 9.5 | 0.2 | 8 | 57.3 | 0.2 | 132.9 | 5.8 |
| 5 | sc-low | 7773 | 18 Nov. 2008 | 24 | 0.2 | 0.2 | 6.6 | 0.2 | 12.2 | 92.8 | 0.2 | 107 | 5.5 |
| 5 | sc-low | 7773 | 18 Nov. 2008 | 48 | 0.2 | 90.9 | 9 | 0.2 | 13.9 | 46.4 | 0.2 | 78.5 | 10.4 |
| 3 | sc-low | 7840 | 25 Nov. 2008 | −1 | 35.1 | 1794.1 | 207 | 1682.1 | 3.9 | 128 | 64.4 | 32.9 | 32.3 |
| 3 | sc-low | 7840 | 25 Nov. 2008 | 2 | 149.6 | 1403.9 | 299.9 | 1713 | 1.2 | 166.2 | 74.5 | 55.6 | 33.4 |
| 3 | sc-low | 7840 | 25 Nov. 2008 | 6 | 59.2 | 1295.3 | 212.5 | 1259.4 | 4.2 | 132 | 56.2 | 7.8 | 42.8 |
| 3 | sc-low | 7840 | 25 Nov. 2008 | 24 | 0.2 | 611.7 | 105.3 | 631 | 1.8 | 64.2 | 27.6 | 0.2 | 16.1 |
| 3 | sc-low | 7840 | 25 Nov. 2008 | 48 | 1.6 | 598.7 | 98.1 | 524.9 | 0.8 | 55.8 | 23.2 | 0.2 | 24 |
| 4 | sc-low | 7868 | 11 Nov. 2008 | −1 | 8.1 | 0.2 | 21.5 | 0.2 | 7.7 | 0.2 | 0.6 | 25.9 | 10.5 |
| 4 | sc-low | 7868 | 11 Nov. 2008 | 2 | 13.1 | 0.2 | 18.4 | 0.2 | 8.2 | 1.2 | 0.1 | 18.6 | 9.1 |
| 4 | sc-low | 7868 | 11 Nov. 2008 | 6 | 0.2 | 0.2 | 13.8 | 0.2 | 3.6 | 0.2 | 0.2 | 10.9 | 0.2 |
| 4 | sc-low | 7868 | 11 Nov. 2008 | 24 | 0.2 | 0.2 | 11.5 | 0.2 | 21.6 | 0.2 | 0.2 | 3.1 | 0.2 |
| 4 | sc-low | 7868 | 11 Nov. 2008 | 48 | 0.2 | 0.2 | 14.2 | 0.2 | 13.1 | 0.2 | 0.4 | 4.4 | 3.5 |
| 2 | sc-low | 7934 | 4 Nov. 2008 | −1 | 97.3 | 354.5 | 95.6 | 0.2 | 50.6 | 253.7 | 18.5 | 273.2 | 47.6 |
| 2 | sc-low | 7934 | 4 Nov. 2008 | 2 | 57.8 | 346.7 | 65.4 | 0.2 | 43 | 210.2 | 16.3 | 208.2 | 42.4 |
| 2 | sc-low | 7934 | 4 Nov. 2008 | 6 | 95.3 | 488.4 | 128.6 | 0.2 | 27.9 | 182.2 | 22.6 | 239.4 | 37.6 |
| 2 | sc-low | 7934 | 4 Nov. 2008 | 24 | 130.7 | 542.3 | 191.4 | 0.2 | 35.8 | 295 | 34.6 | 322.5 | 48.9 |
| 2 | sc-low | 7934 | 4 Nov. 2008 | 48 | 140.8 | 938.5 | 156.8 | 0.2 | 24 | 192.3 | 46.7 | 213.3 | 31.4 |
| 1 | sc-low | 8053 | 4 Nov. 2008 | −1 | 0.2 | 0.2 | 0.2 | 0.2 | 0.6 | 0.2 | 0.2 | 7.5 | 0.2 |
| 1 | sc-low | 8053 | 4 Nov. 2008 | 2 | 0.2 | 0.2 | 0.2 | 0.2 | 0.2 | 0.2 | 0.2 | 0.2 | 0.2 |
| 1 | sc-low | 8053 | 4 Nov. 2008 | 6 | 0.2 | 0.2 | 0.2 | 0.2 | 1.3 | 0.2 | 0.2 | 0.2 | 0.2 |
| 1 | sc-low | 8053 | 4 Nov. 2008 | 24 | 0.2 | 0.2 | 0.2 | 0.2 | 0.3 | 0.2 | 0.2 | 0.2 | 0.2 |
| 1 | sc-low | 8053 | 4 Nov. 2008 | 48 | 0.2 | 0.2 | 0.2 | 0.2 | 4.8 | 0.2 | 0.2 | 0.2 | 0.2 |
| 3 | iv-high | 7840 | 2 Dec. 2008 | −1 | 68.7 | 0.2 | 26 | 0.2 | 0.9 | 17.2 | 1.4 | 47.1 | 34.6 |
| 3 | iv-high | 7840 | 2 Dec. 2008 | 2 | 28.8 | 0.2 | 4.7 | 0.2 | 0.8 | 10.2 | 0.2 | 30.6 | 28.8 |
| 3 | iv-high | 7840 | 2 Dec. 2008 | 6 | 41.4 | 0.2 | 9.8 | 0.2 | 1 | 7.4 | 0.2 | 22.7 | 24.3 |
| 3 | iv-high | 7840 | 2 Dec. 2008 | 24 | 47 | 0.2 | 18.5 | 0.2 | 8.6 | 22.2 | 0.6 | 43.4 | 35.9 |
| 3 | iv-high | 7840 | 2 Dec. 2008 | 48 | 26.6 | 0.2 | 0.2 | 0.2 | 4.3 | 3.5 | 0.2 | 45.2 | 28.1 |
| 6 | iv-low | 7675 | 2 Dec. 2008 | −1 | 0.2 | 0.2 | 0.2 | 0.2 | 6.9 | 0.2 | 0.2 | 0.2 | 10.9 |
| 6 | iv-low | 7675 | 2 Dec. 2008 | 2 | 0.2 | 0.2 | 0.2 | 0.2 | 4.6 | 0.2 | 0.2 | 0.2 | 0.2 |
| 6 | iv-low | 7675 | 2 Dec. 2008 | 6 | 0.2 | 0.2 | 0.2 | 0.2 | 1.8 | 0.2 | 0.2 | 0.2 | 2.3 |
| 6 | iv-low | 7675 | 2 Dec. 2008 | 24 | 0.2 | 0.2 | 1.4 | 0.2 | 6.3 | 0.2 | 0.2 | 0.2 | 12.3 |
| 6 | iv-low | 7675 | 2 Dec. 2008 | 48 | 0.2 | 0.2 | 2.2 | 0.2 | 13.1 | 0.2 | 0.2 | 10.1 | 12.5 |

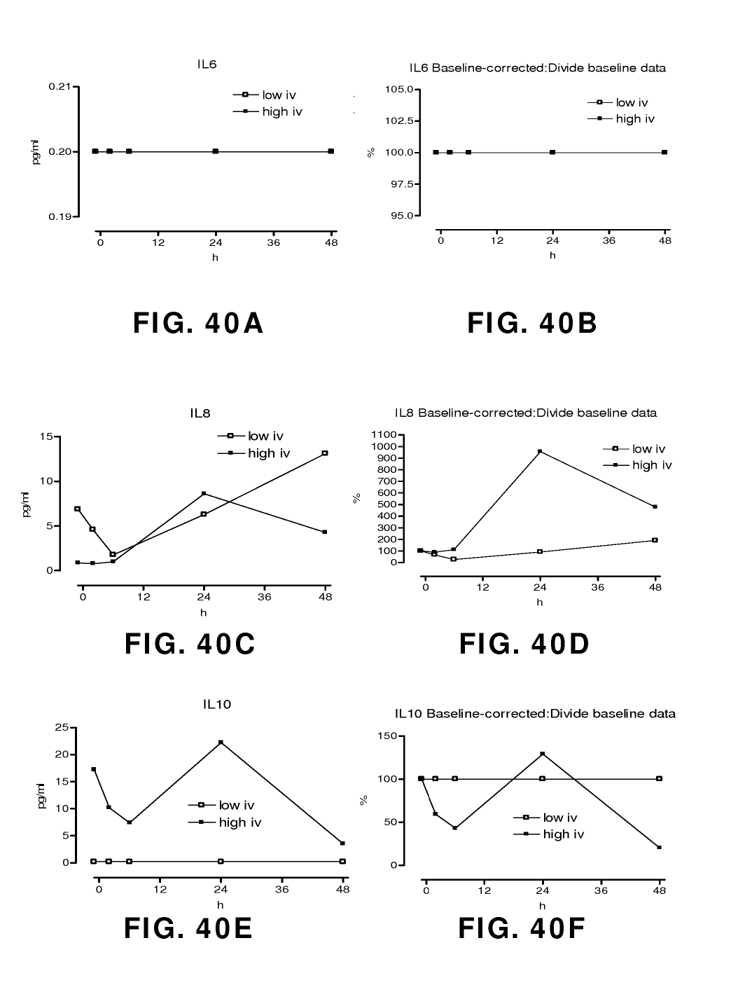

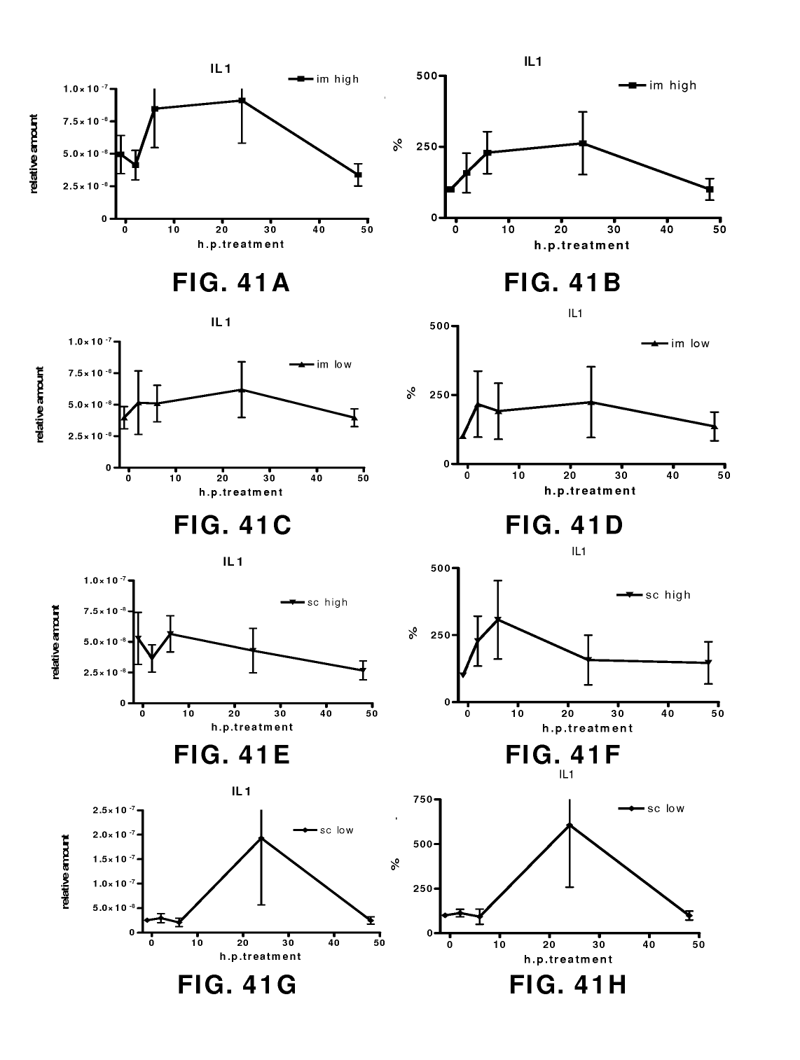

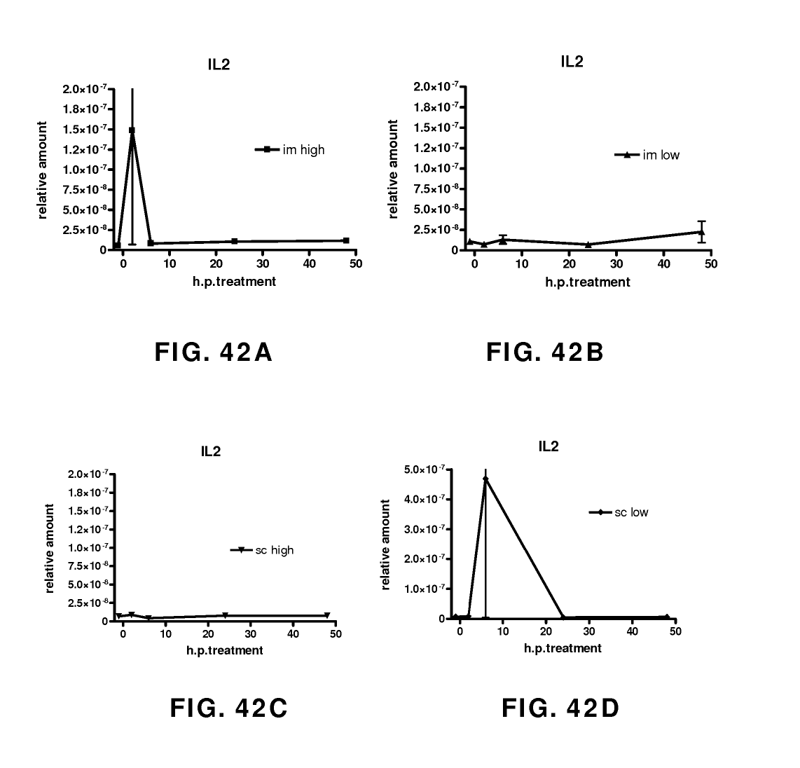

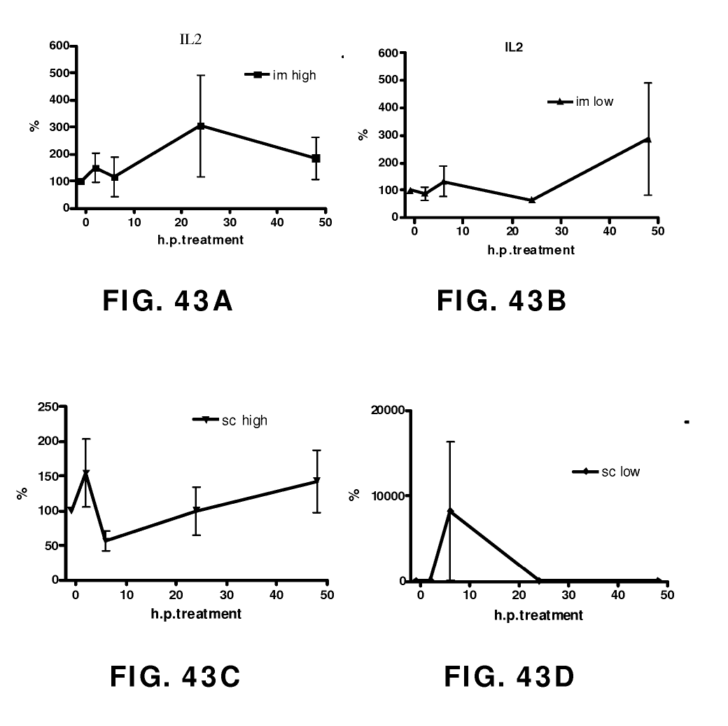

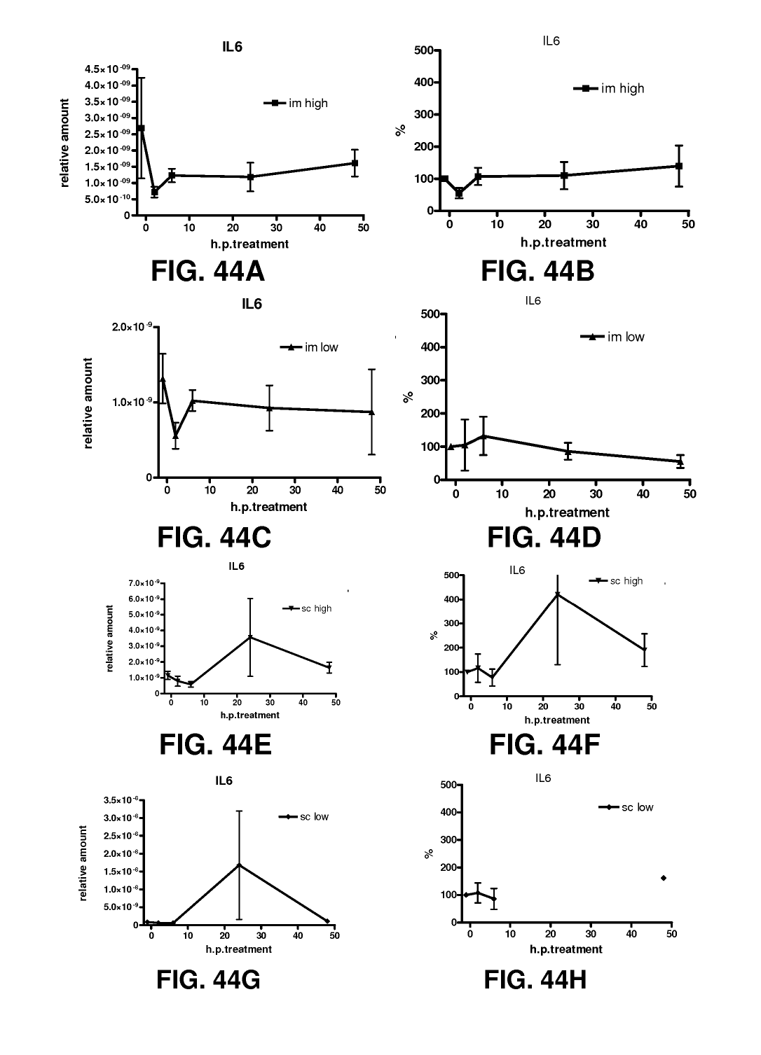

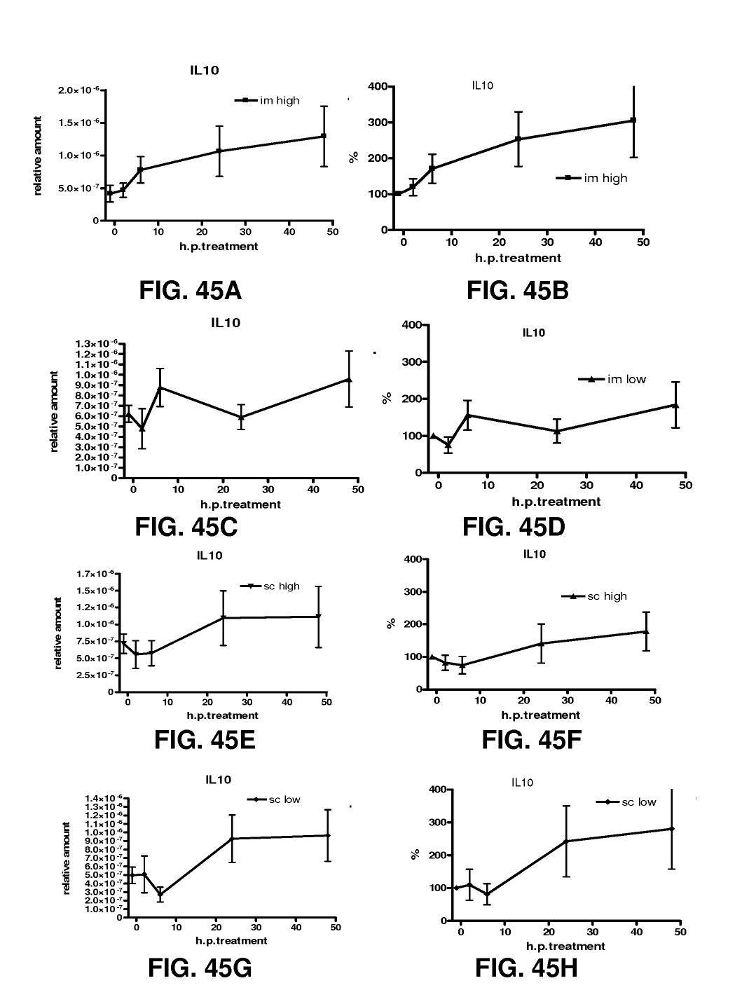

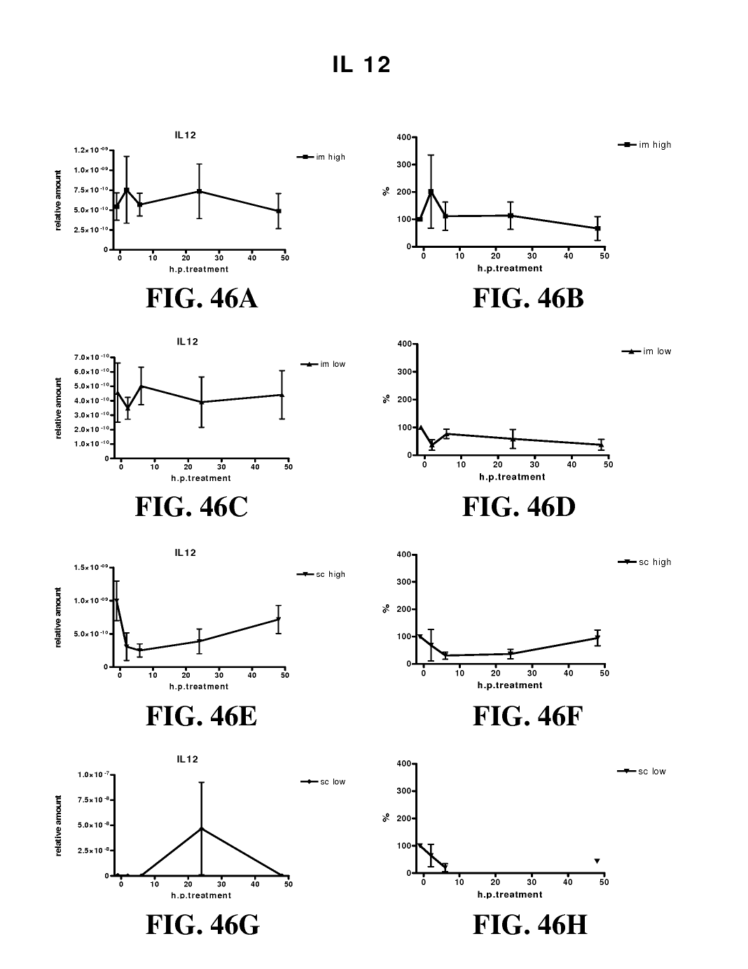

The relative amount of mRNA of cytokine IL-1, IL-2, IL-6, IL-10 and IL-12 in blood cells are presented in FIGS. 41-48. An increase of IL-1 to up to 300% compared to the base line value was observed after intramuscular administration of the high and low dose and after subcutaneous administration of the high dose. After 48 hours post treatment mRNA content returned to baseline values, a (preliminary) two way ANOVA analysis (time, treatment) revealed a trend for time as a cause of variation (p-value 0.07). No clear effects on the expression level of IL-2 and IL-6 were observed. An increase of the expression level of IL-10 mRNA to 200 or 300% was observed in all treatment groups starting from 6 hours post treatment in pigs after intramuscular administration and observed at 24 hours post treatment in pigs treated subcutaneously. The results of a (preliminary) two-way ANOVA analysis revealed time as a source of variation (p<0.05). No consistent findings were observed for changes in the mRNA expression level of IL-12.

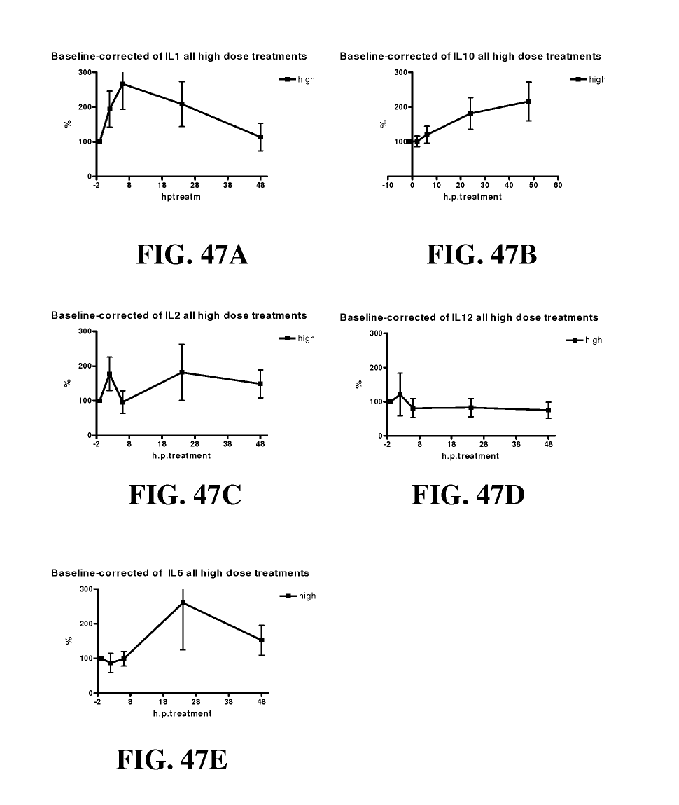

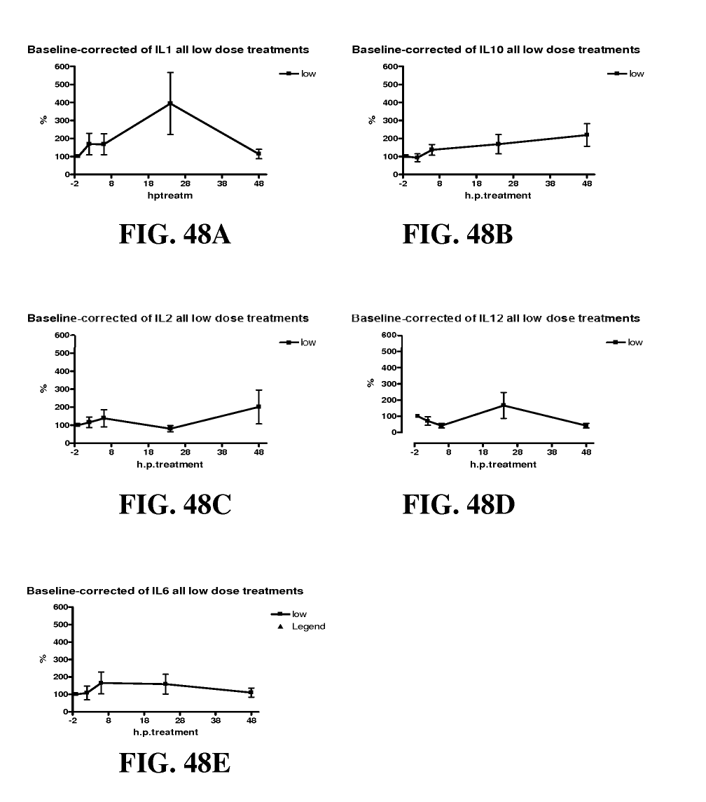

Comparing high and low dose administration regardless of administration route a stronger, dose related effect seems to be present for IL-1 and IL-10 change (FIGS. 47 and 48).

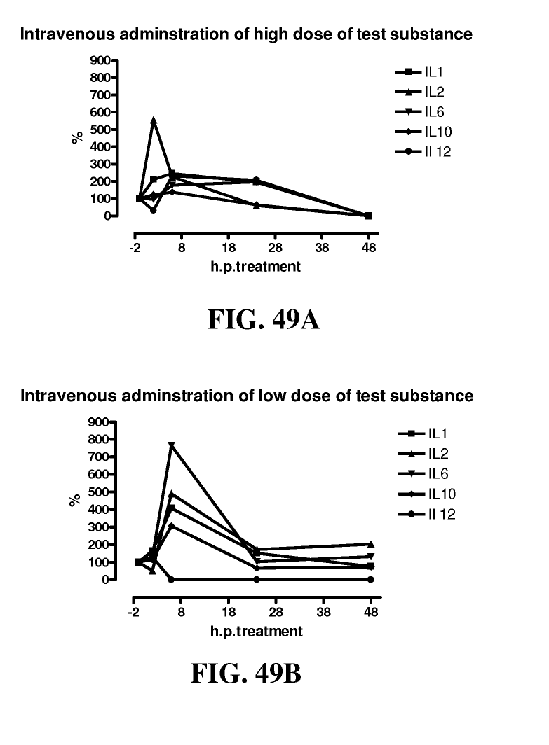

The effects of intravenous administration of a high or low dose of the test substance were examined in one pig respectively and are depicted in FIGS. 49A and B.

In a cross over study effects of the lipid-DNA Complex Seq No 2-F after intramuscular and subcutaneous administration of a high and a low dose were assessed by studying changes in mRNA expression of different cytokines in blood cells, changes in serum cytokine contents, hematological findings, appearance of clinical signs and performance criteria. Whereas no effects were seen on serum cytokine contents for a range of different cytokines, analysis of mRNA expression of IL-1 and IL-10 revealed in circulating blood cells a (dose-dependent) increase after treatment. IL-1 is a pro-inflammatory cytokine, which is produced by and acts on a number of different cell types. Next to the function of a pro-inflammatory mediator, IL-1 is a potent Th2 stimulator. IL-10 is considered to be one of the most important anti-inflammatory cytokines and is a potent inhibitor of Th1 cytokines and known as a deactivator of monocyte/macrophage pro-inflammatory cytokine synthesis.

Treatment did not result in adverse reactions as no clinical signs have been observed after any administration of the test substance. Also no effects on WBC or differential cell count have been observed. It remains inconclusive whether the described changes of the red blood cell count, hemoglobulin and hematocrit are related to the test substance itself or to the administration of the test substance, although stress situations were eliminated as much as possible during the treatment phase by using catheters.

### Example 10: Timing of Immunomodulator Administration

The purpose of this study was to determine if administering Seq No 2-F prior to infection and a subsequent administration of Seq No 2-F is effective against Mannheimia haemolytica infection and BRD.

Seq No 2-F was administered to 40 3-month old Holstein steers one day prior to and one day post inoculation with 60 mL of and 10⁸ CFUs/mL of Mannheimia haemolytica. Necropsy was performed 5 days post-challenge.

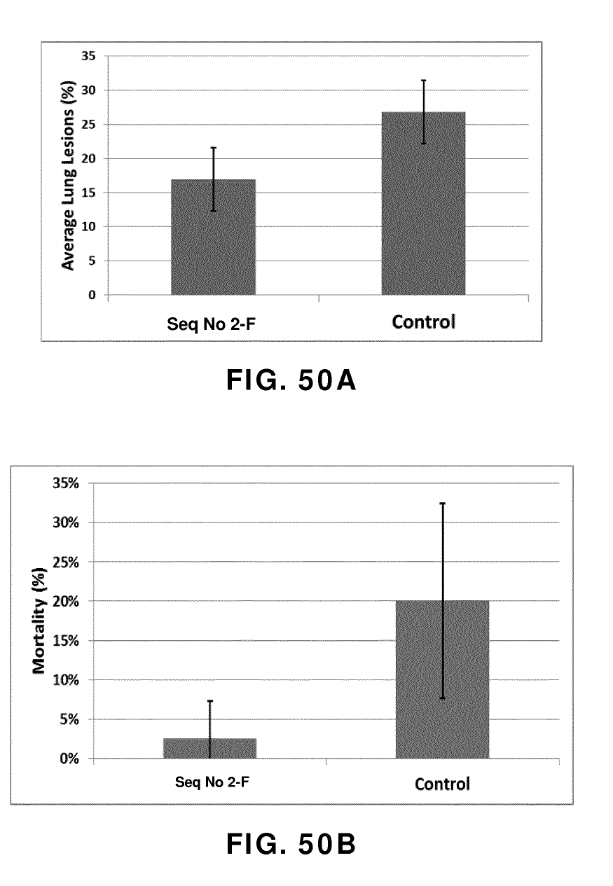

Results show that the percentage of lung lesions in steers receiving treatment one day prior to and one day post inoculation was approximately 10% less compared to controls (approximately 10% and 27%, respectively) (FIG. 50A). Mortality due to BRD was significantly reduced in steers receiving treatment compared to controls. Only 2.5% of treated steer, compared to 20% of control steer, experienced mortality due to BRD (FIG. 50B), which suggests that a first treatment prior to exposure to Mannheimia haemolytica and a second exposure one day after exposure has a protective effect against lung lesions and illness.

### Example 11: Combination Therapy for Cattle Diagnosed with BRD

The purpose of this study was to compare the efficacy and net returns of BRD treatment in cattle with Seq No 2-F, an antibiotic, and a combination therapy comprising Seq No 2-F and the antibiotic.

### Study Population

212 freshly weaned heifers weighing on average between 400 and 500 lbs that were considered high risk for BRD were selected for participation. The heifers were divided into three treatment groups. After diagnosis, the first group (n=78) received 5.7 ml/cwt of Baytril® 100 (enrofloxacin) injected subcutaneously. Baytril® 100 is an antimicrobial agent used in the treatment of BRD. The second group (n=77) received 2 mL of Seq No 2-F delivered intramuscularly. The third group (n=57) received 5.7 ml/cwt of Baytril® 100 delivered subcutaneously and 2 ml of Seq No 2-F delivered intramuscularly. After a three day moratorium post-diagnosis, the heifers were observed until day 60 post-diagnosis.

If, at any time after the three day moratorium, a calf still met the clinical requirements of a BRD diagnosis, the calf would be pulled from the study and administered Draxxin® (tulathromycin). If the calf did not respond to this second treatment, the calf would then be repulled and treated for a third time with Bio-Mycin® 200 (oxytetracycline). After three treatments, the calf would be deemed to have chronic BRD and treatments halted.

### Results

Referring to Table 6, the combination of Seq No 2-F and Baytril® 100 a significantly reduced percentage of BRD case fatalities compared to either the Seq No 2-F alone or Baytril® 100 alone. Average weight gain for the heifers that received the combination therapy also significantly outpaced the comparative single therapy groups. The groups receiving the combined therapy and the Baytril® 100 treatment alone had a lower percentage of calves being repulled for subsequent treatments compared to group receiving only Seq No 2-F. The group receiving the combined therapy also had a lower percentage of chronic BRD compared to the other groups. Although the difference was not statistically significant, only 18.9% of calves in the combination therapy group developed chronic BRD, while 38.7% of calves receiving Seq No 2-F alone developed chronic BRD.

TABLE 6
Individual and Combination Therapy Comparison

| | | Baytril 100 + | |
|---|---|---|---|
| | Baytril 100 | Seq No 2-F | Seq No 2-F |
| Parameter | (n = 78) | (n = 77) | (n-57) |
| BRD repulls | 44.8% | 46.5% | 73.9 |
| BRD Chronicity | 22.10% | 18.9% | 38.7 |
| BRD case-fatality | 10.0% | 4.4% | 22.3% |
| ADG (deads out) lbs/day | 1.8 | 1.7 | 1.4 |
| Avg wt gain (deads in) lbs | 24.3 | 61.80 | −103.9 |
| Avg wt gain (deads out) lbs | 87.00 | 94.30 | 24.6 |

These results show that combining the present immunomodulator with an antibiotic effective against BRD can improve herd health. Furthermore, these improvements were shown to have an economic advantage of approximately $110 per head, thus providing producers with a cost-effective robust combination therapy.

### Example 12: Metaphylaxis for Cattle with Medium Risk of BRD

The purpose of this study was to determine if Seq No 2-F was inferior to the commercially available antibiotic, Micotil, when administered to control BRD in feedlot cattle.

### Study Population

Weaned beef calves with an average weight of 590 lbs and determined to be at medium risk for BRD were selected for this study. At the time of diagnosis, the first group (n=1002) were administered 2 ml/cwt of Micotil, and the second group (n=1002) was administered 2 ml of Seq No 2-F. Including a three day moratorium, the calves were observed for 56 days.

### Results

Morbidity, mortality, average daily gain (ADG), dry matter intake (DMI), and feed to weight gain ratio were measured and compared between the study groups. To be considered inferior required a 10% difference in BRD morbidity between the two groups. The difference in the cumulative incidence of BRD morbidity between the group receiving Seq No 2-F and the group receiving Micotil was approximately 6% with an error margin of less than +1.3%. Referring to Table 7, nearly all other clinical parameters measured were also not significantly different. The one exception, was that the time to BRD treatment was significantly less in the group receiving Seq No 2-F than the group receiving Micotil.

TABLE 7
Inferiority Analysis Results

| Parameter | Micotil | Seq No 2-F | p-value |
|---|---|---|---|
| Time to BRD Dx | 28.1 | 22.6 | <0.0001 |
| BRD repulls | 17.9% | 11.1% | 0.5929 |
| BRD Chronicity | 27.90% | 29.1% | 0.9942 |
| Overall BRD Mortality | 0.44% | 0.50% | 0.7643 |
| BRD case-fatality | 3.95% | 2.99% | 0.7287 |
| ADG | 2.96 | 2.91 | 0.6759 |
| DMI | 12.96 | 12.81 | 0.3768 |
| Feed: Gain | 4.50 | 4.55 | 0.7302 |

This study shows that the immunomodulator Seq No 2-F was determined to not be inferior to the antibiotic Micotil. Because resistance to antibiotic therapy is a potential risk to herd health and to the sustainability of livestock operations, effective non-antibiotic antimicrobial therapies are a valuable option for producers.

### Example 13: Combination Therapy for Cattle Diagnosed with BRD

The purpose of this study was to compare the effectiveness of Draxxin®, Draxxin® and Seq No 2-F, or Seq No 2-F alone in cattle diagnosed with BRD.

### Study Group

For this study, weaned beef calves with an average weight of 625 lbs were divided in to three treatment groups. The first group was administered 1.1 ml/cwt of Draxxin® subcutaneously at the time of diagnosis. The second group was administered 1.1 ml/cwt of Draxxin® subcutaneously and 2 ml of Seq No 2-F intramuscularly, and the third group was administered 2 ml of Seq No 2-F intramuscularly. A 3 day moratorium followed treatment, and the study concluded 56 days after the diagnosis.

### Results

Referring to Table 8, the combination of Seq No 2-F and Draxxin® significantly reduced BRD morbidity compared to treatment with Seq No 2-F alone (21.8% and 45.8%, respectively). The combination therapy also resulted in lower BRD morbidity compared to treatment with Draxxin® alone (21.8% and 29.2%, respectively). The percentage of cattle with chronic BRD was lower for the combination therapy (2.9%) than for either the Seq No 2-F therapy alone (8.9%) or the Draxxin® therapy alone (4.0%). Fatalities attributable to BRD were also decreased in the group receiving the combination therapy compared to treatment with Seq No 2-F alone or treatment with Draxxin® alone.

The combination therapy was also associated with greater production than the single therapy approaches. Average daily gain and average weight gain were higher for the combination therapy, which resulted in an economic advantage of approximately $34/head compared to the Draxxin® only treatments.

TABLE 8
Individual and Combination Therapies

| | | Draxxin + | |
|---|---|---|---|
| Parameter | Draxxin | Seq No 2-F | Seq No 2-F |
| Time to BRD Dx (days) | 18.8 | 19.2 | 14.6 |
| BRD morbidity (%) | 29.2 | 21.8 | 45.8 |
| BRD repulls (%) | 37.2 | 43.9 | 48.2 |
| BRD chronicity (%) | 4.0 | 2.9 | 8.9 |
| BRD case-fatality (%) | 1.3 | 0.7 | 2.7 |
| ADG (deads in) lbs/day | 3.2 | 3.3 | 2.8 |
| ADG (deads out) lbs/day | 3.3 | 3.5 | 2.9 |
| Avg wt gain (deads in) lbs | 181.1 | 189.9 | 156.3 |
| Avg wt gain (deads out) lbs | 187.0 | 194.1 | 164.6 |

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above products, compositions, and methods without departing from the scope of the invention, it is intended that all matter contained in the above description and shown in the accompanying drawings shall be interpreted as illustrative and not in a limiting sense.

What is claimed is:

1. A method of eliciting an immune response in a recipient subject comprising:
a. administering an immunomodulator composition to the subject, wherein the immunomodulator composition comprises a nucleic acid sequence comprising at least one immunostimulatory CpG motif, at least one non-immunostimulatory CpG motif, and a cationic liposome; and,
b. activating immune surveillance receptors, wherein the immune surveillance receptors activate signaling pathways involved in an innate immune response.

2. The method of claim 1, wherein the signaling pathway includes signaling molecules selected from the group consisting of TLR9, TLR21, cGAS, IFI16, DDX41, DNA-PK, DAI, Mre11, LRRFIP1, AIM2, RNA-Polymerase III/RIG-I, STING, ASC, NFκB, AP1, MAPK, IRF3, and combinations thereof.

3. A method of stimulating an immune response in a subject comprising:
a. administering to the subject an immunomodulator composition, wherein the immunomodulator composition comprises a nucleic acid sequence having at least 80% sequence homology with the sequence of SEQ ID NO: 1 and a liposome delivery vehicle and,
b. activating immune surveillance receptors, wherein the immune surveillance receptors activate signaling pathways involved in an innate immune response.

4. The method of claim 3, wherein the liposome delivery vehicle comprises lipids selected from the group consisting of multilamellar vesicle lipids and extruded lipids.

5. The method of claim 3, wherein the liposome delivery vehicle comprises pairs of lipids selected from the group consisting of N-[1-(2,3-dioleyloxy)propyl]-N,N,N-trimethylammonium chloride (DOTMA) and cholesterol; N-[1-(2,3-dioleoyloxy)propyl]-N,N,N-trimethylammonium chloride (DOTAP) and cholesterol; 1-[2-(oleoyloxy)ethyl]-2-oleyl-3-(2-hydroxyethyl)imidazolinium chloride (DOTIM) and cholesterol; and dimethyldioctadecylammonium bromide (DDAB) and cholesterol.

6. The method of claim 3, wherein administration is selected from the group consisting of intravenously, intramuscularly, intramammary, intradermal, intraperitoneal, subcutaneously, by spray, by aerosol, in ovo, mucosally, transdermally, by immersion, orally, intraocularly, intratracheally, and intranasally.

7. The method of claim 3, wherein the immunomodulator composition further comprises a biological agent.

8. The method of claim 3, wherein the administration is before exposure to an infectious agent.

9. The method of claim 3, wherein the administration is after exposure to an infectious agent.

10. The method of claim 3, wherein the subject is selected from the group consisting of mammal species, aquaculture species, and avian species.

11. The method of claim 3 further comprising a pharmaceutically acceptable carrier.

12. A method of stimulating an immune response in a subject comprising:
a. administering to the subject an immunomodulator composition, wherein the immunomodulator composition comprises a nucleic acid sequence having at least 80% sequence homology with the sequence of SEQ ID NO: 4 and a liposome delivery vehicle; and,
b. activating immune surveillance receptors, wherein the immune surveillance receptors activate signaling pathways involved in an innate immune response.

13. The method of claim 12, wherein the liposome delivery vehicle comprises lipids selected from the group consisting of multilamellar vesicle lipids and extruded lipids.

14. The method of claim 12, wherein the liposome delivery vehicle comprises pairs of lipids selected from the group consisting of N-[1-(2,3-dioleyloxy)propyl]-N,N,N-trimethylammonium chloride (DOTMA) and cholesterol; N-[1-(2,3-dioleoyloxy)propyl]-N,N,N-trimethylammonium chloride (DOTAP) and cholesterol; 1-[2-(oleoyloxy)ethyl]-2-oleyl-3-(2-hydroxyethyl)imidazolinium chloride (DOTIM) and cholesterol; and dimethyldioctadecylammonium bromide (DDAB) and cholesterol.

15. The method of claim 12, wherein administration is selected from the group consisting of intravenously, intramuscularly, intramammary, intradermal, intraperitoneal, subcutaneously, by spray, by aerosol, in ovo, mucosally, transdermally, by immersion, orally, intraocularly, intratracheally, and intranasally.

16. The method of claim 12, wherein the immunomodulator composition further comprises a biological agent.

17. The method of claim 16, wherein the biological agent is selected from the group consisting of immune enhancer proteins, immunogens, vaccines, antimicrobials, and any combination thereof.

18. The method of claim 12, wherein the administration is before exposure to an infectious agent.

19. The method of claim 12, wherein the administration is after exposure to an infectious agent.

20. The method of claim 12, wherein the subject is selected from the group consisting of mammal species, aquaculture species, and avian species.

21. The method of claim 12 further comprising a pharmaceutically acceptable carrier.

22. A method of modulating the STING signaling pathway to elicit an immune response in a recipient subject comprising:
a. administering an immunomodulator composition to the subject, wherein the immunomodulator composition comprises a nucleic acid sequence comprising at least one immunostimulatory CpG motif, at least one non-immunostimulatory CpG motif, and a cationic liposome.

23. A method of modulating an immune response in a subject comprising:
a. administering to the subject an immunomodulator composition, wherein the immunomodulator composition comprises a nucleic acid sequence having at least 80% sequence homology with the sequence of SEQ ID NO: 4 and a liposome delivery vehicle; and,
b. activating immune surveillance receptors, wherein the immune surveillance receptors activate signaling pathways involved in modulating an immune response.

24. The method of claim 23, wherein the immune surveillance receptors activate signaling pathways involved in stimulating an innate immune response.

25. The method of claim 23, wherein the immune surveillance receptors activate signaling pathways involved in stimulating an acquired immune response.

26. The method of claim 23, wherein the immune surveillance receptors activate signaling pathways involved in suppressing an inflammatory immune response.

27. A method for increasing weight gain of cattle diagnosed with bovine respiratory disease comprising:
administering an antimicrobial agent to the subject in combination with an immunomodulator composition comprising a nucleic acid sequence having at least 80% homology with SEQ ID NO: 1 and a lipid delivery vehicle, wherein the combination increases weight gain in the subject.

28. The method of claim 27, wherein the antimicrobial agent is enrofloxacin.

29. A method for increasing weight gain of cattle diagnosed with bovine respiratory disease comprising:
administering an antimicrobial agent to the subject in combination with an immunomodulator composition comprising a nucleic acid sequence having at least 80% homology with SEQ ID NO:4 and a lipid delivery vehicle, wherein the combination increases weight gain in the subject.

30. The method of claim 27, wherein the antimicrobial agent is enrofloxacin.
